(19) United States
(12) Patent Application Publication
(10) Pub. No.: US 2017/0314044 A1
(43) Pub. Date: Nov. 2, 2017

(54) ADENOVIRUS CONSTRUCTS AND METHODS

(71) Applicant: REGENTS OF THE UNIVERSITY OF MINNESOTA, MINNEAPOLIS, MN (US)

(72) Inventors: Julia Davydova, Minneapolis, MN (US); Masato Yamamoto, Minneapolis, MN (US); Lisa Koodie, Minneapolis, MN (US)

(21) Appl. No.: 15/584,394

(22) Filed: May 2, 2017

Related U.S. Application Data
(60) Provisional application No. 62/330,467, filed on May 2, 2016.

(51) Int. Cl. C12N 15/86; C07K 14/005; A61K 35/761; G01N 33/50; C12N 7/00; A61K 38/00

(52) U.S. Cl. CPC C12N 15/86; G01N 33/5091; C12N 7/00; C07K 14/005; A61K 35/761; A61K 38/00; C12N 2710/10333; C12N 2710/10343

(57) ABSTRACT

This disclosure describes oncolytic adenovirus constructs and methods on using the constructs. Generally, the oncolytic constructs includes at least a partial deletion of E3, a heterologous polynucleotide encoding a heterologous polypeptide in place of the at least partial deletion of E3, and a cell-specific regulatory polynucleotide operationally linked to the heterologous polynucleotide.

### CROSS-REFERENCE TO RELATED APPLICATION

This application claims priority to U.S. Provisional Patent Application No. 62/330,467, filed May 2, 2016, which is incorporated herein by reference.

### GOVERNMENT FUNDING

This invention was made with government support under 5R01-CA174861 awarded by the National Institutes of Health. The government has certain rights in the invention.

### SEQUENCE LISTING

This application contains a Sequence Listing electronically submitted to the United States Patent and Trademark Office via EFS-Web as an ASCII text file entitled “11005490101SequenceListing_ST25.txt” having a size of 98 kilobytes and created on May 2, 2017. Due to the electronic filing of the Sequence Listing, the electronically submitted Sequence Listing serves as both the paper copy required by 37 CFR §1.821(c) and the CRF required by §1.821(e). The information contained in the Sequence Listing is incorporated by reference herein and does not go beyond the disclosure in the International Application as filed.

### SUMMARY

This disclosure describes oncolytic adenovirus (OAd) constructs and methods on using the constructs. Generally, the oncolytic constructs include at least a partial deletion of the adenoviral region E3, a heterologous polynucleotide encoding a heterologous polypeptide in place of the at least partial deletion of E3, and a cell-specific regulatory polynucleotide operationally linked to the heterologous polynucleotide.

In some embodiments, the heterologous polynucleotide encodes a therapeutic polypeptide effective for treating cancer. In some of these embodiments, the therapeutic polypeptide can include a polypeptide that kills tumor cells. In other embodiments, the therapeutic polypeptide can include a polypeptide that sensitizes tumor cells to a primary cancer therapy such as radiotherapy, chemotherapy, and/or immunotherapy.

In some embodiments, the heterologous polynucleotide encodes a diagnostic polypeptide. In some of these embodiments, the diagnostic polypeptide binds to a radioisotope.

In some embodiments, the cell-specific regulatory polynucleotide can include a tumor-specific promoter.

In some embodiments, the construct can further include a genetic modification that increases transduction efficiency of the adenovirus construct into target cells. I some of these embodiments, the genetic modification can include a polynucleotide that encodes a fiber knob polypeptide of adenovirus type 3 (Ad3).

In some embodiments, the construct can lack a functional adenovirus death protein (ADP). In other embodiments, the construct can include an ADP coding region.

The above summary of the present invention is not intended to describe each disclosed embodiment or every implementation of the present invention. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

### BRIEF DESCRIPTION OF THE FIGURES

The patent or application file contains at least one drawing or photograph executed in color. Copies of this patent or patent application publication with color drawing(s) or photographs(s) will be provided by the Office upon request and payment of the necessary fee.

FIG. 1. Oncolytic adenovirus expressing luciferase coding region from the E3 region can track distant metastases non-invasively and improve survival rate. (A) Non-invasive imaging. Luc signal (virus) corresponds to the location of fluorescent markers (cancer). (B) Bioluminescence imaging originating from Ad visualized small metastases. (C) Improved survival rate with the virus.

FIG. 2. Therapeutic efficacy and survival rate of standard antitumor therapies with oncolytic adenovirus expressing Interferon alpha (IFN-α). (A) Tumor inhibition was observed when radiation and 5-FU were joined with Ad-mediated IFN therapy. (B) Significant improvement in survival was observed only when 5-FU and/or radiation was combined with Ad-IFN.

FIG. 3. Structure of tumor-selective NIS-expressing oncolytic adenoviruses. (A) Cox2-ΔE3/ΔADP-NIS: Cox2-controlled vector without adenovirus death protein; (B) Cox2-ΔE3/ADP-NIS: Cox2-controlled vector with adenovirus death protein.

FIG. 4. Structure of NIS-expressing Oncolytic Adenoviruses. (A) Wt-ΔE3/ΔADP -NIS: wild type replication (non-selective) vector without adenovirus death protein; (B) Wt-ΔE3/ADP-NIS: wild type replication (non-selective) vector with adenovirus death protein.

FIG. 5. Confirmation of vector design. Polymerase chain reaction assay show the presence of adenoviral death protein (ADP) and the absence of adenovirus death protein in the ΔE3 vectors.

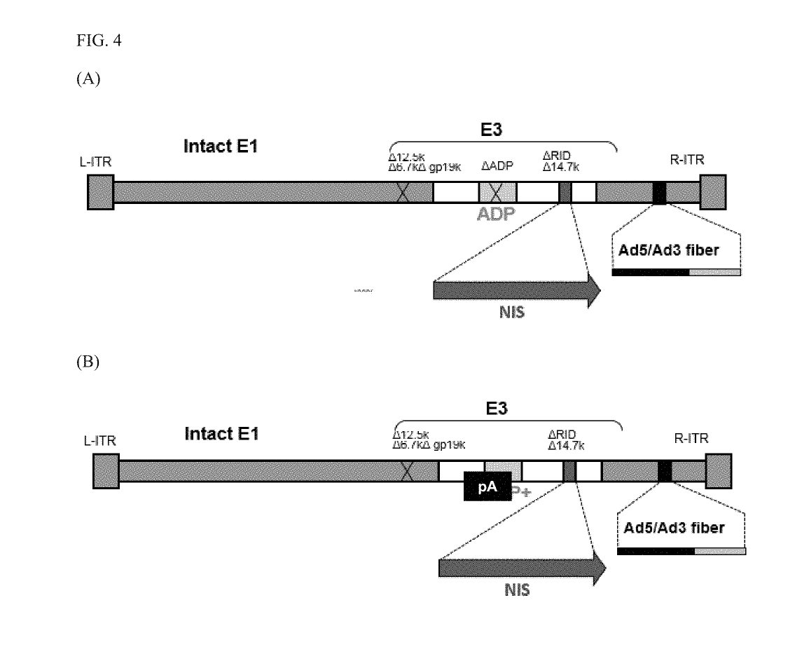

FIG. 6. The ADP+ vector is more cytolytic than noADP counterpart at earlier time points in multiple pancreatic cancer cell lines and showed improved viral replication and spreading compared to wild type control (Ad5Wt) as determined using the crystal violet assay. The noADP construct, however, resulted in cell death at later time points, catching up with the ADP counterparts. Cox2-controlled oncolytic adenoviruses (ADP+ and noADP) did not produce any cell death in Cox2-negative control-BT474 cells, confirming vector selectivity.

FIG. 7. All NIS-expressing vectors efficiently produced glycosylated NIS multimers in pancreatic cancer cell lines as early as two days post-infection with 1 viral particle per cell as determined using Western blot analysis. NoADP vectors produced greater amounts of NIS proteins when compared to their ADP+ counterparts or replication incompetent Ad-CMV-NIS in various pancreatic cancer cells tested.

FIG. 8. All vectors increased the percentage of NIS positive cells over 2-6 days as determined using flow cytometry. However, infections with noADP vectors resulted in a higher percentage of NIS-positive cells when compared to ADP+ vectors or non-replicating Ad-CMV-NIS virus. Panc-1 human pancreatic cancer cell line.

FIG. 9. NIS protein localization and expression patterns in pancreatic cancer cells using immunocytochemistry. (A) All vectors resulted in stable cell surface expression of NIS, however noADP vectors produced more NIS-expressing cells. (B) NIS expression increased on day 3 and further by day 5. (C) The noADP vector produced significantly higher amount of NIS-positive-cell foci than its ADP-positive counterparts, suggesting that noADP vectors is better for radioisotope uptake. All images at 40× magnification.

FIG. 10. Radioiodine uptake using multiple pancreatic cancer cells in vitro. All vectors (1 vp/cell) increased radioiodine uptake over control uninfected or non-replicative Ad-CMV-NIS at two days post-infection. However, infection of pancreatic cancer cells with noADP vectors resulted in a greater radioiodine (¹²³I) uptake than ADP+ counterparts supporting its use as a novel diagnostic tool.

FIG. 11. Ex vivo viral replication in human pancreatic cancer patient tissues. Cox2 promoter-controlled NIS-expressing vector (Cox2-ΔE3/ADP-NIS) showed no replication in human normal pancreatic tissues and robust replication in adenocarcinoma tissues comparable to that of non-selective wild type replication control.

FIG. 12. Ex vivo radioiodine uptake in human pancreatic tissues. Cox2 promoter-controlled NIS-expressing vector (Cox2-ΔE3/ADP-NIS) showed very limited ¹²⁵I uptake in normal tissue (right) with strong iodine uptake in cancerous cells (left, circled).

FIG. 13. In vivo imaging studies in human pancreatic cancer xenografts in a mouse model. SPECT-CT images show that both low viral dose (A) and high viral doses (B) of the ADO-lacking oncolytic adenovirus (Cox2-ΔE³/ΔADP-NIS) achieved a more sustained radioisotope uptake (⁹⁹mTc^{O4-}) when compared to the positive control, AdCMV-NIS, and ΔE3/ADP-NIS counterpart using a human pancreatic cancer xenograft mouse model. (A) 4×10⁹ VP/tumor, single injection; (B) 4×10¹⁰ VP/tumor, single injection.

FIG. 14. Therapeutic effect of virus treatments: Volumes of tumors in data from FIG. 13. (A) High dose (4×10¹⁰ VP/tumor), single injection; (B) Low dose (4×10⁹ VP/tumor), single injection.

FIG. 15. Immunocytochemistry analyses of NIS protein in pancreatic tumor xenografts of virus-treated animals. (A) PBS—negative control, (B) Cox-2ΔE3/ADP-INS; (C) Ad-CMV-NIS positive control; (D) Cox-2ΔE3/ΔADP-INS. The noADP vector (D) produced more NIS-positive regions in tumors and cell foci than its ADP+ counterpart (B) and the positive control (C), further supporting utility as a diagnostic for pancreatic cancer. 4×10⁹ VP/tumor.

FIG. 16. Oncolytic adenovirus-induced NIS expression in a Patient Derived Xenograft (PDX) Model. Quantification after immuno-cytochemical analyses of Ad-hexon (green) and NIS protein (red) in human pancreatic adenocarcinoma patient derived tumors tissues of virus-treated animals. (A) NIS expression after ADP+ vector therapy resulted in a punctate NIS expression pattern more reminiscent of cytoplasmic localization. (B) Bar graph showing similar Ad-hexon levels, suggesting viral infection and spreading were similar between noADP and ADP+. (C) The noADP vector produced more NIS with a cell membrane pattern of localization. (D) The noADP vectors expressed significantly more NIS than ADP+therapy and this accounts for the observed differences in the radioisotope uptake studies.

FIG. 17. Oncolytic adenovirus-induced NIS expression in a Patient Derived Xenograft (PDX) Model. Immunocytochemistry analyses of Cytokeratin 19 and Ad-hexon using human-patient-derived adenocarcinoma tissues from virus-treated animals. 4×10⁹VP/tumor. Human tumors stained positive for Cytokeratin 19 (red) and co-localized with Ad-hexon (green), confirming that our viral constructs targeted pancreatic adenocarcinoma cells. All images are from one patient.

FIG. 18. Non-invasive imaging of adenovirus-induced NIS expression in a patient-derived xenograft (PDX) model of pancreatic adenocarcinoma using tissues from four different patients. Cox2-ΔE3/ΔADP-NIS sustained an earlier, higher intensity, and longer lasting radioiodine uptake compared to other NIS-expressing vectors, further supporting its use as a diagnostic tool for pancreatic cancer.

FIG. 19. Volumes of tumors from the PDX model data in FIG. 18. The PDX tumors were treated with a single dose of adenovirus (4×10¹⁰ VP/tumor).

FIG. 20. Radioiodine-131 (¹³¹I) uptake in a human pancreatic adenocarcinoma xenograft model. Cox2-ΔE3-noADP-NIS therapy supported the greatest ¹³¹I uptake.

FIG. 21. Therapeutic effect of oncolytic adenovirus-facilitated iodine therapy in a human pancreatic adenocarcinoma xenograft model. The ADP+ vector (Cox2-NIS-(+)ADP) and ΔADP counterpart (Cox2-NIS-(−)ADP) in combination with a single therapeutic dose of ¹³¹I both reduced tumor progression.

FIG. 22. The vector structure of ΔE3/ΔADP oncolytic adenovirus as a platform for gene delivery. TSP—Tumor Specific Promoter.

FIG. 23. Plasmid map of ΔE3/ΔADP oncolytic adenovirus (Ad5/Ad3-Cox2-ΔE3/ΔADP-NIS).

### DETAILED DESCRIPTION OF ILLUSTRATIVE EMBODIMENTS

This disclosure describes a novel oncolytic virus-based technology platform that has therapeutic and diagnostic utility. This technology is based on a genetically modified oncolytic adenovirus that can produce a level of transgene expression in tumor sites that can allow imaging of cancer cells and/or cancer therapy. For example, the oncolytic adenovirus can facilitate immunotherapy by expressing an immunostimulatory cytokine such as, for example, interferons, (IFNs, e.g., IFN-α, IFN-β; interleukins (ILs, e.g., IL-2, IL-6, IL-7, IL-8, IL-12); granulocyte-macrophage colony stimulating factor (GM-CSF), and/or therapeutic antibodies (e.g., anti-CTLA4 and anti-PD1). Viral-based immunotherapies can initiate local tumor lysis and/or stimulate immune response against cancer cells. Alternatively, the oncolytic adenovirus can be designed to express a therapeutic and/or imaging gene—e.g., the sodium iodine symporter (NIS), which can induce uptake of radioactive iodine by tumor cells. NIS expression allows noninvasive imaging of cancer with different radioisotopes (e.g., 123I, ¹²⁴I, ⁹⁹mTcO₄, ¹⁸⁸ReO₄) and facilitates therapy with radioiodine ¹³¹I. Another application of the technology is its ability to monitor tumor development, disease staging, and/or adenovirus biodistribution by expressing a reporter transgene such as, for example, the green fluorescent protein (GFP or EGFP), red fluorescent protein (RFP), fluorescent reporter mCherry, herpes simples virus type 1 thymidine kinase (HSV1-tk) or mutant HSV1-sr39tk, somatostatin receptor (SSTR), a radiolabeled antibody against carcinoembryonic antigen (CEA), or secreted embryonic alkaline phosphatase (SEAP).

In some embodiments, the oncolytic adenovirus lacks the adenovirus death protein (ADP), the deletion of which improves gene expression. Expression of the transgene is replication-dependent: a therapeutic transgene is expressed in tumor cells with each round of adenovirus replication. Moreover, the oncolytic adenovirus can be designed to replicate—and therefore express the transgene—only in cancer cells.

Tumor-specific promoters can be used to control adenovirus replication. One extensively used method to develop tumor-targeted oncolytic adenovirus is to control viral replication with a tumor-specific promoter. Several promoters possess a suitable profile for tumor-specific oncolytic adenovirus development such as, for example, cyclooxygenase-2 (Cox2), heparin-binding growth/differentiation factor midkine (MK) promoter, mucin 1 (Muc1) promoters for pancreatic cancer; probasin (PB) and prostate—specific antigen (PSA) promoters for prostate cancer; and other candidates promoters that share the characteristic of being overexpressed in cancer while possessing the “liver off” profile—e.g., human telomerase catalytic subunit (hTERT), gastrin releasing peptide (GRP), vascular endothelium growth factor (VEGF), secretory leukocyte protease inhibitor (SLPI), fms-like tyrosine kinase 1(FLT-1), and caveolin-1 promoters. These tumor-specific promoters can be active in primary and established cancer cells while exhibiting very low activity in normal tissues. In some cases, the tumor-specific promoter can exhibit low activity in normal liver tissue, which can be the organ of most concern involving ectopic adenoviral vector gene expression. Systemically-delivered oncolytic adenoviruses demonstrated a low level of toxicity and liver sequestration, indicating the feasibility of tumor-specific-promoter-based oncolytic adenoviruses for systemic cancer therapy.

In some embodiments, the oncolytic adenovirus can include a genetic modification that increases transduction efficiency of the oncolytic adenovirus in cancer cells. The genetic modification can include, for example, modification of the viral capsid. Many cancer cells are resistant to human adenovirus type 5 (Ad5) infection due to profoundly low expression of the primary adenovirus receptor (coxsackie-adenovirus receptor, CAR). Changing oncolytic adenovirus tropism via genetic modification of the viral capsid proteins enables CAR-independent entry. Thus, replacing the fiber knob of Ad type 5 with the fiber knob of Ad type 3 (Ad3) retargets the modified oncolytic adenovirus to the Ad3 receptor (identified as CD46 and human desmoglein-2 receptor), which is typically highly expressed in cancer cells. The Ad5/Ad3 modification of the Ad fiber increases the viral infectivity in many different cancer cells in vitro and in vivo (e.g. esophageal adenocarcinoma, pancreatic cancer, lung cancer, melanoma, colon cancer, breast cancer, and ovarian cancers).

In some embodiments, an increase in antitumor effects can be achieved by incorporating an anti-tumor transgene into the genome of an oncolytic adenovirus. This local transgene expression, coupled with strong vector spread, can significantly advance oncolytic virotherapy for cancer. One strategy for expressing a transgene upon Ad replication in tumor cells uses an antitumor therapeutic transgene—e.g., NIS—placed into the Ad E3 region of the oncolytic adenovirus. Some embodiments can overexpress Adenoviral Death Protein (ΔE3/+ADP): ADP facilitates viral release and leads to more efficient intratumoral spread of the virus. In some embodiments, the oncolytic adenovirus lacks the Adenovirus Death Protein (ADP), the deletion of which improves transgene expression. Transgene expression from the E3 region is controlled by the adenoviral major late promoter and is therefore consistent with the replication cycle, a property that can be further exploited to monitor the adenovirus.

A ΔE3-based oncolytic adenovirus may be designed to express any suitable transgenes from the adenoviral E3 region. Exemplary transgenes that can be expressed in this manner include, for example, therapeutic transgenes such as IFN-α, INF-β, IL-2, IL-6, IL-7, IL-8, IL-12, GM-C SF, anti-CTLA4 antibody, anti-PD1 antibody, or NIS; or imaging/reporter transgenes such Luc, RFP, GFP, EGFP, mCherry, SSTR, TKsr39, CEA, SEAP, or NIS.

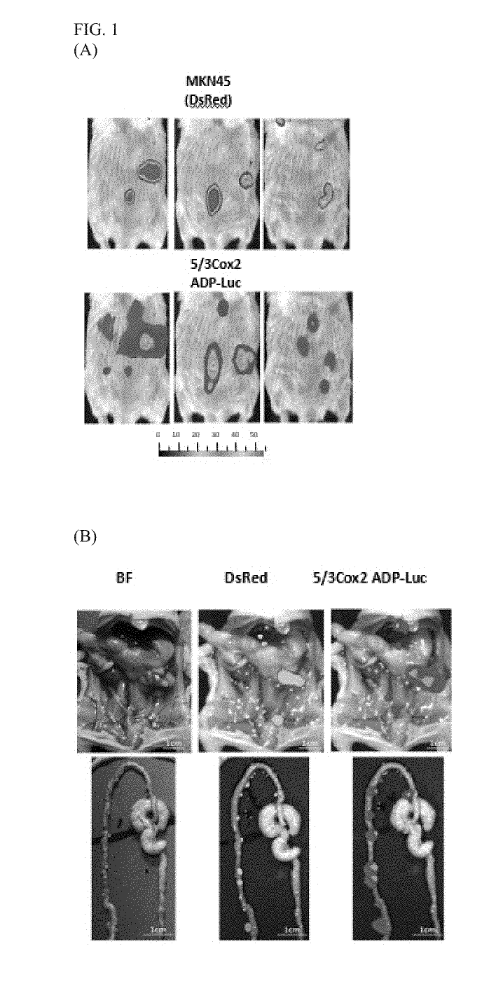

FIG. 1 shows data demonstrating the improved ability of the oncolytic adenovirus to specifically overexpress a gene of interest in the target tumors and/or detect viral replication after systemic and local viral administration in tumor xenografts and distant metastases (FIG. 1).

In some embodiments, the oncolytic adenovirus can be used to express a therapeutic compound. One exemplary embodiment involves the use of an oncolytic adenovirus designed to deliver interferon-based therapy. In one embodiment of the ΔE3/+ADP oncolytic adenovirus system, the oncolytic adenovirus was designed to express human interferon-a gene (Ad-IFN). IFN-α can exhibit one or more properties—e.g., antitumor effects through immunomodulation, direct inhibition of tumor cell growth, antiangiogenesis, radio sensitization and/or chemosensitization—that can exploited in a cancer therapy regimen. IFN-α-expressing oncolytic adenovirus can stimulate immune effect against pancreatic cancer cells and can be used as a therapeutic agent for treating pancreatic cancer.

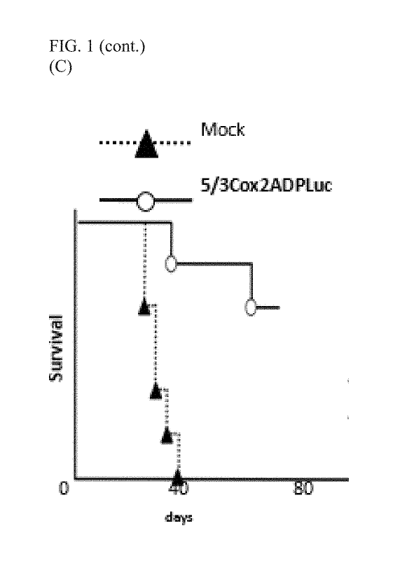

In addition, a multidrug analysis revealed that a combination of oncolytic Ad-IFN with chemotherapeutics (e.g., fluorouracil (5FU), gemcitabine, cisplatin) and radiotherapy (e.g., X-ray) killed human and hamster pancreatic cancer cells significantly better than either of the single treatments in vitro and in vivo. These results support the impact of oncolytic adenovirus-mediated IFN-α to sensitize chemotherapy and radiation for pancreatic cancer (FIG. 2).

In some embodiments, the oncolytic adenovirus can provide both therapeutic and diagnostic utility. In one exemplary embodiment, the oncolytic adenovirus can be designed to express a transgene that encodes the sodium iodine symporter (NIS), a human protein that is involved in concentrating iodine in the thyroid. Natural expression of NIS in the thyroid has been exploited as a way to achieve radiotherapy of benign thyroid disease and thyroid cancer for 70 years. This therapy has proven to be safe and effective. NIS expression can be directly imaged at high sensitivity using noninvasive radioiodine scanning and routine nuclear medicine techniques. This imaging requires only a minimal diagnostic dose of radioisotope. Because the uptake of radioiodine in tissues can be quantitated, both the distribution and the quantity of NIS expression can be easily and safely monitored noninvasively. For example, combined SPECT/CT or PET/CT scanners allow the fusion of functional data with high-resolution anatomical (CT) data to provide accurate localization and quantitative estimate of radioactivity. NIS can promote cellular uptake of different radioisotopes, e.g. ¹²³I, ¹²⁴I, ⁹⁹mTcO₄, ¹⁸⁸ReO₄, and ¹³¹I. Moreover, NIS can induce uptake of radioactive iodine by tumor cells and enable radiotherapy with ¹³¹I, thus synergistically enhancing the effect of viral oncolysis.

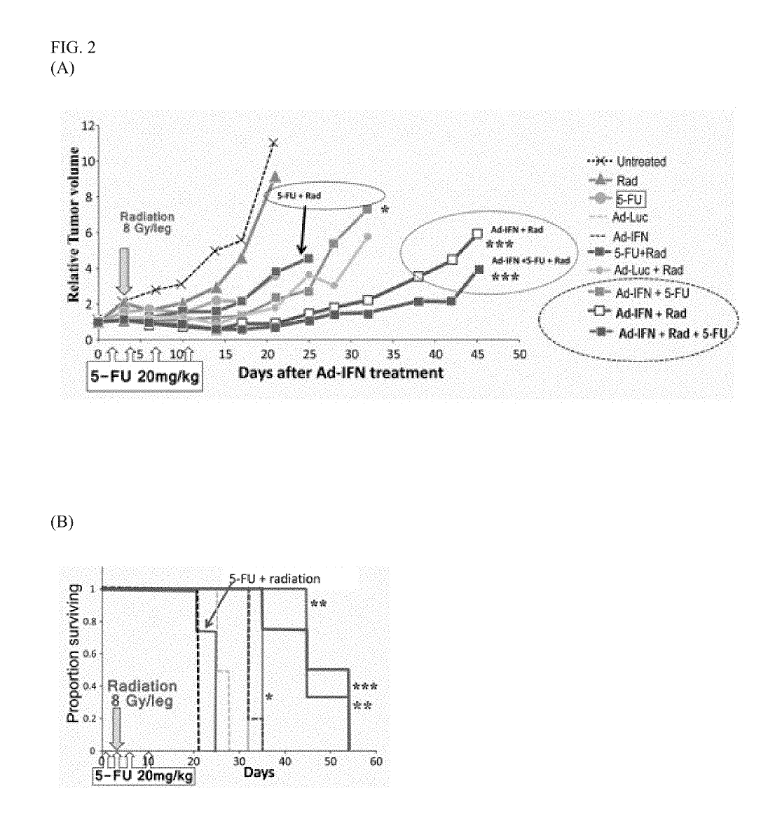

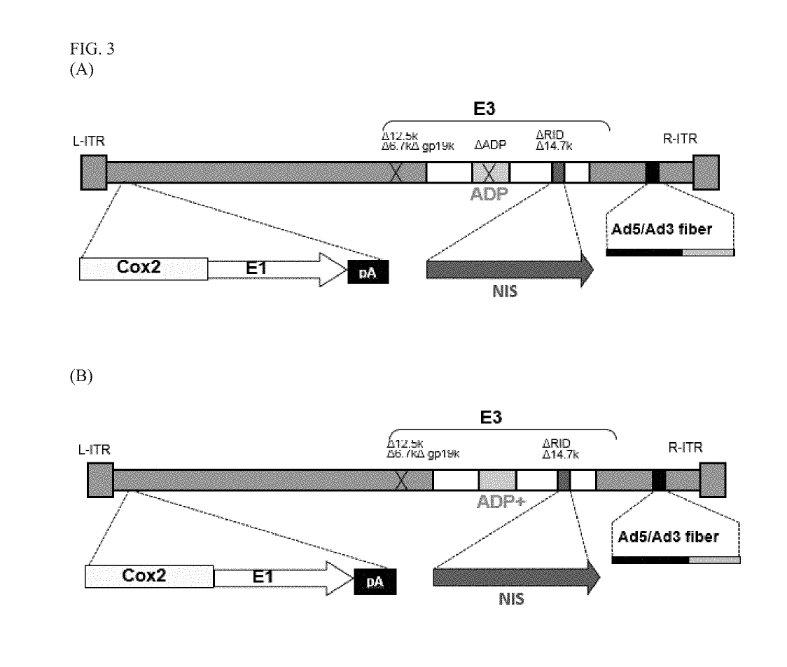

The NIS-expressing oncolytic adenovirus was constructed based on a ΔE3-based adenovirus. Although adenoviral death protein (ADP) enhances oncolysis and viral release in solid tumors, oncolysis also can negatively affect gene expression from the adenovirus and reduce uptake of radiotracer. Two NIS-expressing adenovirus constructs, lacking ADP, were generated. One included a wild type replication vector (Wt-ΔE3/ΔADP-NIS) and the other was Cox2-controlled (Cox2-ΔE3/ΔADP-NIS) (FIG. 3, FIG. 4). These constructs were compared to their respective ADP-positive counterparts (Wt-ΔE3/ADP-NIS and Cox2-ΔE3/ADP-NIS) in pancreatic cancer in vivo and in vitro models.

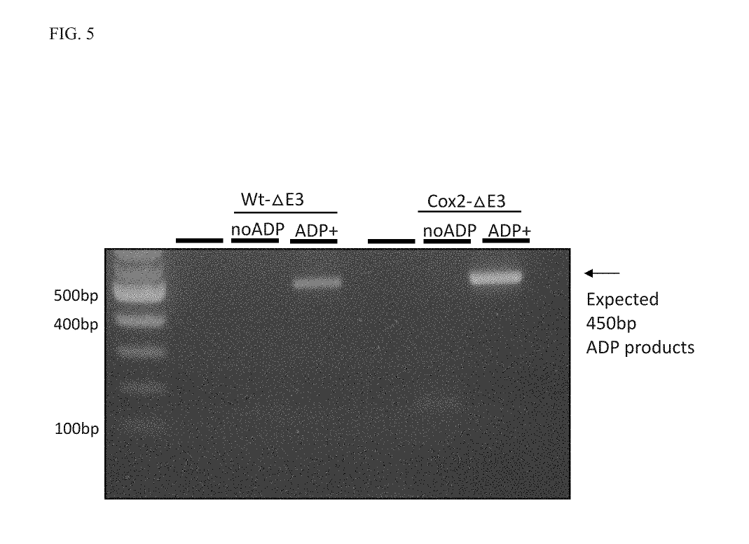

The ability of a NIS-expressing oncolytic adenovirus to kill cancer cells in vitro was evaluated using pancreatic cancer cell line models. The ADP+ construct was significantly more cytolytic than noADP counterpart at earlier time points (FIG. 6). The noADP construct, however, was able to produce cell death by day 10 (FIG. 6). Both Cox2-controlled oncolytic adenoviruses (ADP+ and noADP) did not produce cell death in Cox2-negative control cell line, confirming vector selectivity (FIG. 6).

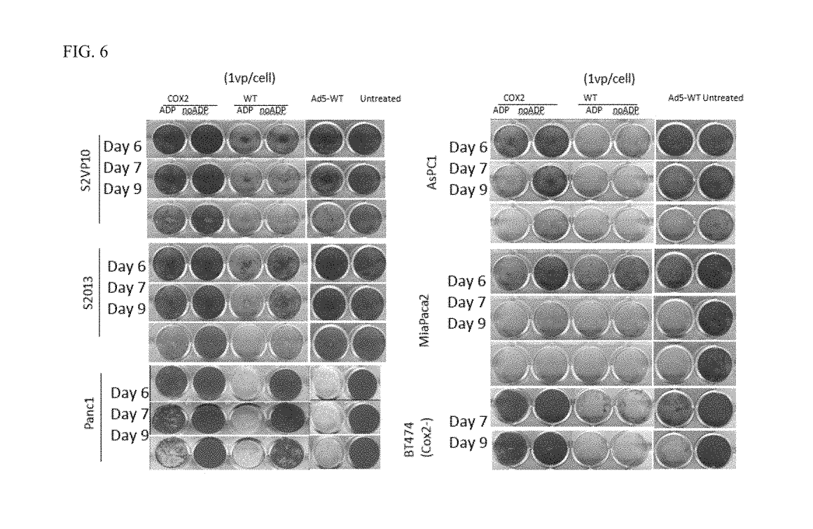

Oncolytic adenovirus expression of NIS in pancreatic cancer cell lines was evaluated by Western Blot. All NIS-OAds efficiently produce glycosylated NIS multimers as early as two days post-infection. However, infection with no-ADP vectors resulted in a significantly greater radioiodine uptake (¹²⁵I) compared to ADP+ viruses (FIG. 7).

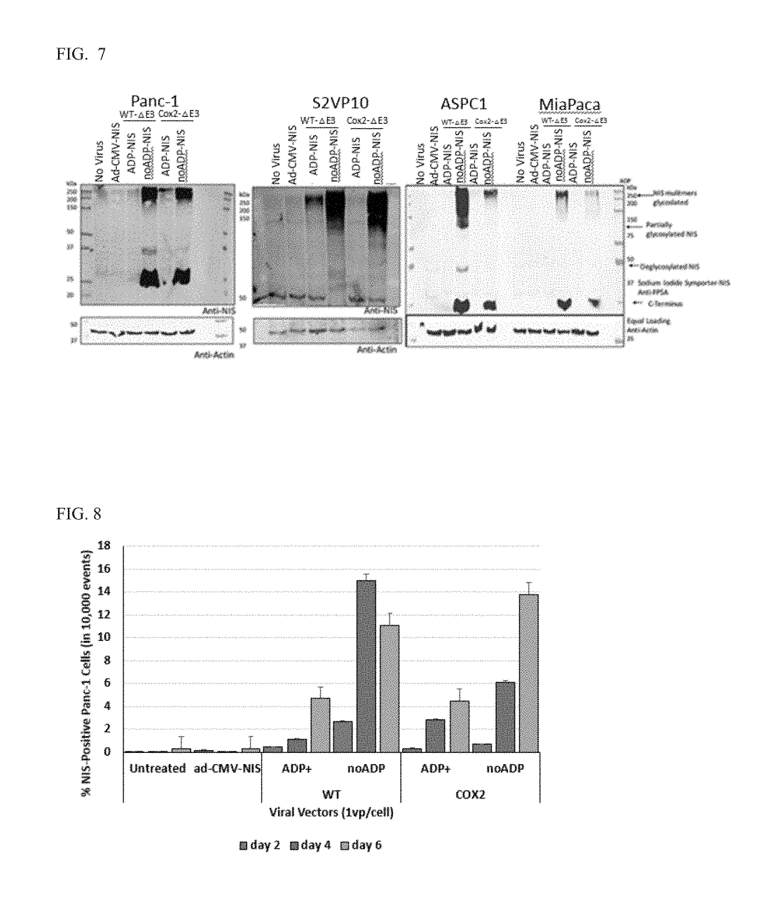

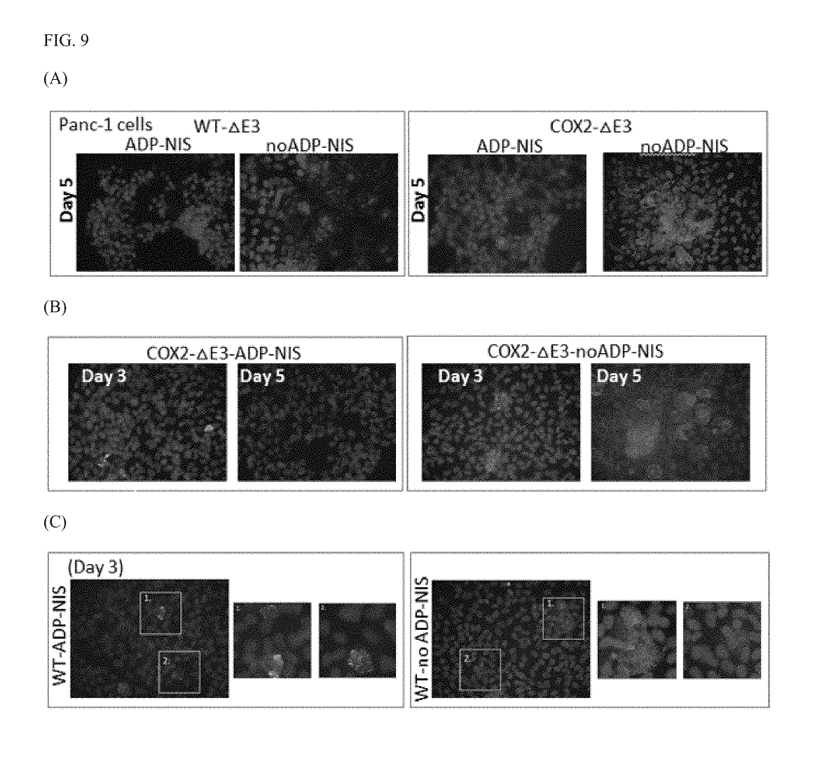

Similarly, flow cytometry revealed that infection with noADP vectors resulted in a higher percentage of NIS-positive cells when compared to ADP+ infections in vitro (p<0.05) (FIG. 8). Immunocytochemistry analyses demonstrated stable cell surface expression of NIS on day 3 after infection pancreatic cancer cells with all NIS-expressing vectors and this increased to a greater extent on day 5 (FIG. 9). However, noADP vector produced significantly higher amount of NIS-positive-cell foci than its ADP-positive counterpart (FIG. 9).

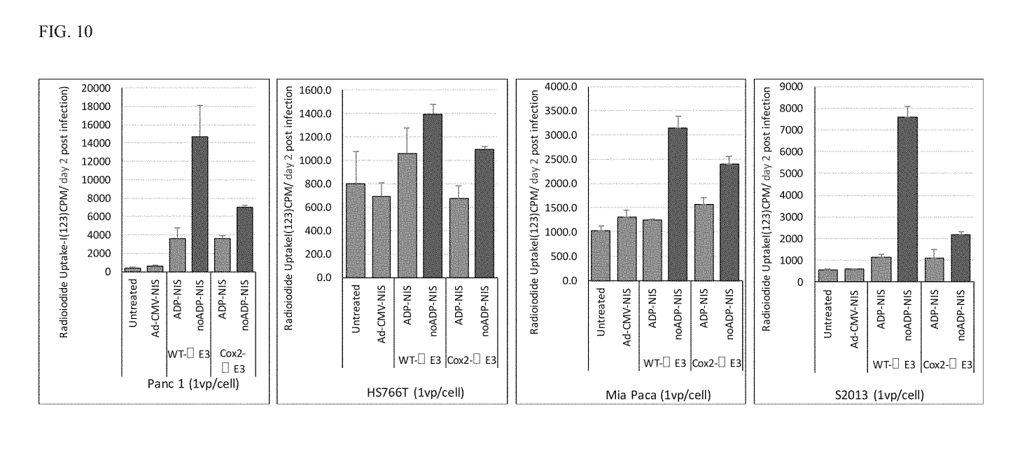

FIG. 10 shows that in vitro infection of Panc-1 cells with NIS-expressing OAd-noADP resulted in significantly greater radioiodine (¹²³¹I) uptake than with its ADP-positive counterpart (p<0.05).

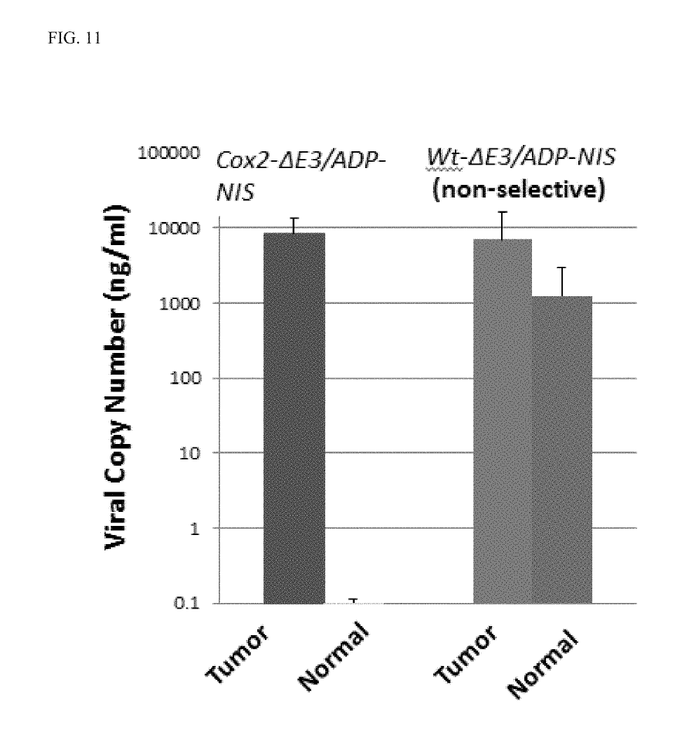

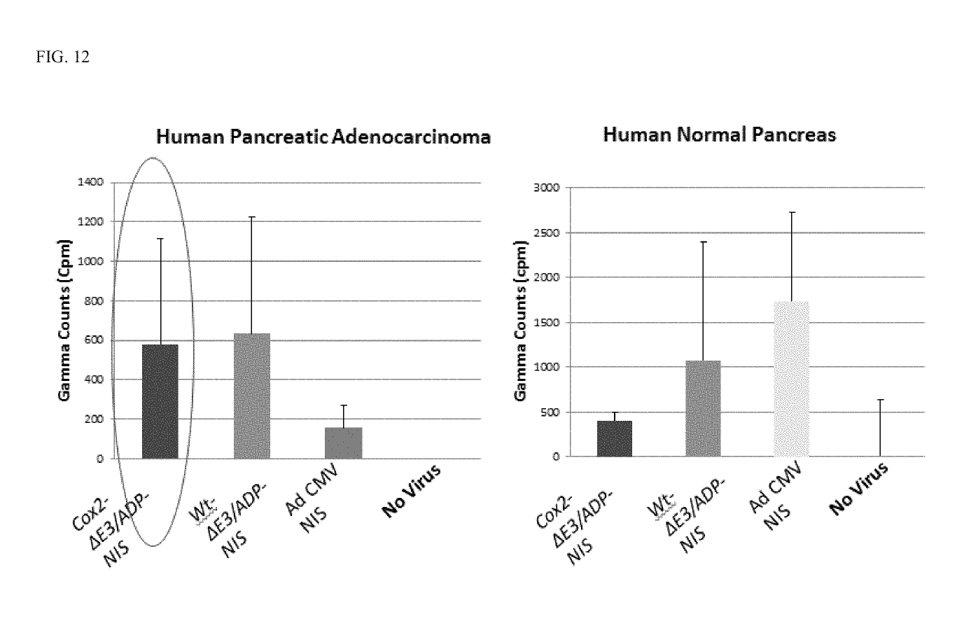

Primary cell cultures from patient cancer tissues may not necessarily accurately reflect in vivo activity, however. For example, isolating the primary cells can result in the loss of tissue structure, a change of cell character, and/or dropout of some cell populations, each of which can alter the character of the cell compared to a comparable cell in a live tissue. Therefore, to analyze the oncolytic adenovirus functionality and safety with live human tissues, ex vivo tissue slicer technology was used to prepare live tissues from de-identified specimens of biopsy-proven human pancreatic ductal adenocarcinoma and adjacent normal pancreas. Data provided in FIG. 11 and FIG. 12 the selectivity of the tumor-selective vector: infection of fresh human patient tissues with a tumor-selective Cox2-ΔE3/ADP-NIS confirmed a high level of viral DNA in pancreatic adenocarcinoma samples, but no virus replication in normal pancreas (FIG. 11). The Cox2-ΔE3/ADP-NIS vector showed no ¹²⁵I uptake in normal human pancreatic tissue and strong uptake in human pancreatic adenocarcinoma tissue slices (FIG. 12). The replication-defective adenovirus expressing NIS (AdCMV-NIS) was included as a positive control in this and the following experiments (ADCMV-NIS is currently being evaluated in a Phase 1 clinical trial for prostate cancer therapy and imaging).

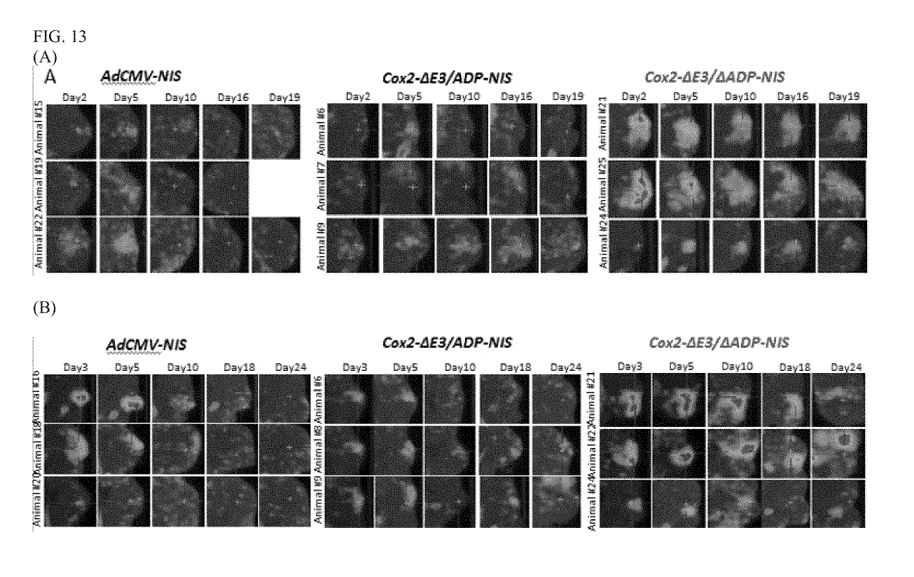

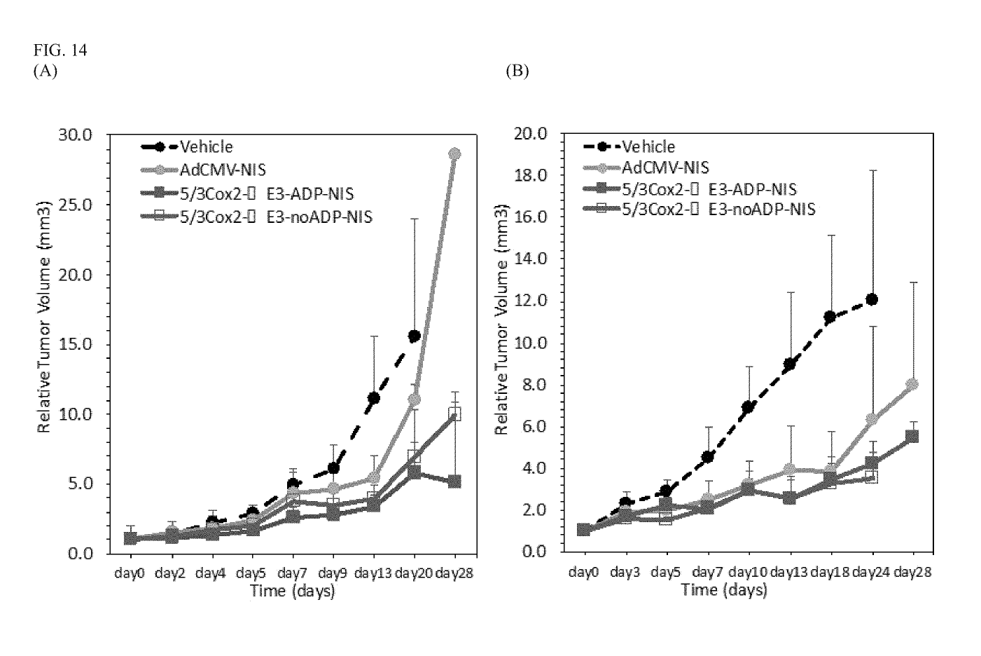

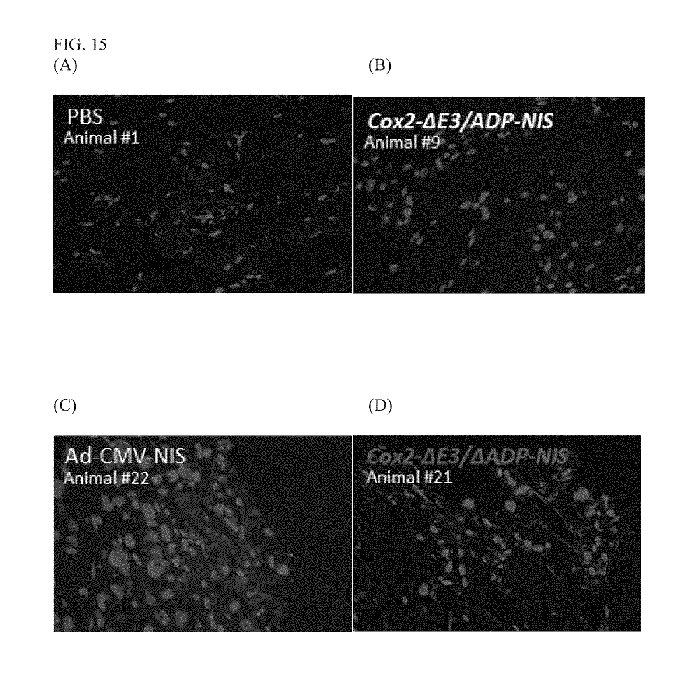

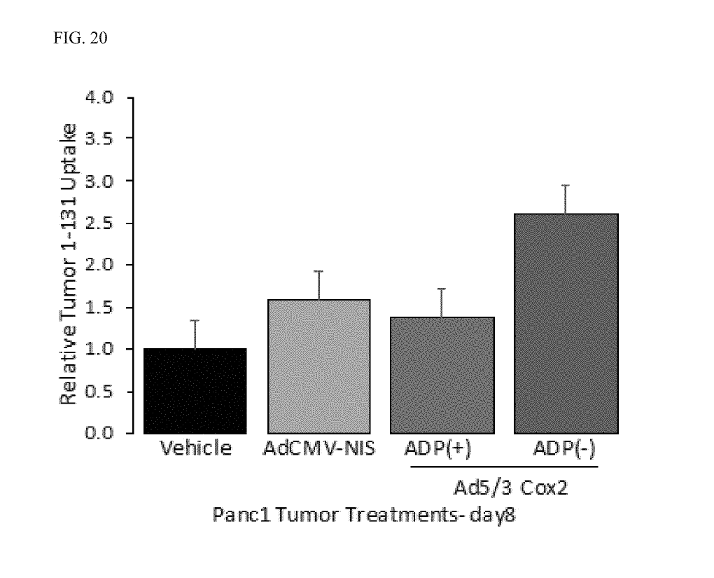

The Cox2-ΔE3/ΔADP-NIS construct also showed increased radiotracer uptake than its ADP+ counterpart and ADCMV-NIS positive control in various in vivo models (FIG. 13, FIG. 15-18, and FIG. 20). The ability of the oncolytic adenovirus constructs to visualize human pancreatic cancer xenografts in a mouse model was first assessed by monitoring [⁹⁹mTcO₄]⁻ accumulation with SPECT-CT. In two separate studies (low and high viral dose), the Cox2-ΔE3/ΔADP-NIS construct showed a more sustained radioisotope uptake when compared to the positive control, AdCMV-NIS, and ADP-positive counterpart, supporting its utility as a sensitive diagnostic tool for pancreatic cancer (FIG. 13). Of note, reduction in tumor size was comparable between the ADP+ and noADP constructs (FIG. 14).

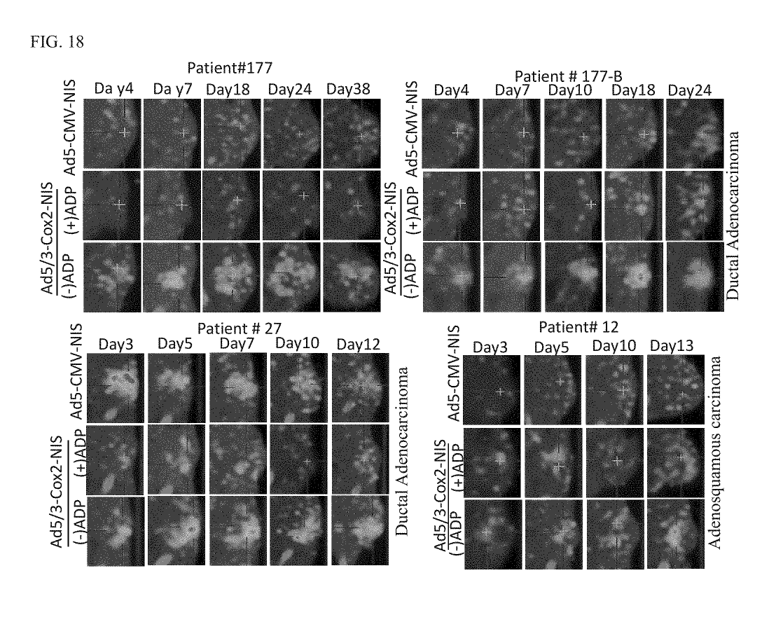

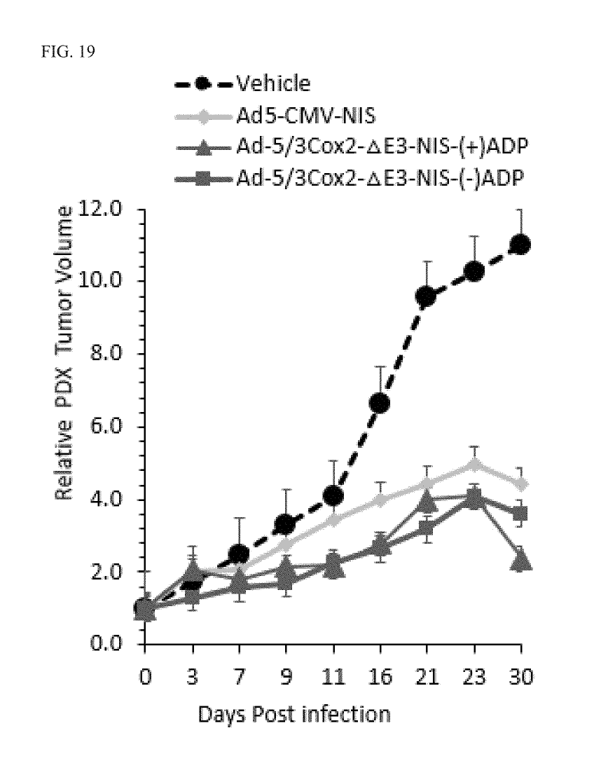

Similarly, evaluation of NIS-expressing adenovirus constructs using a pre-clinical human pancreatic adenocarcinoma patient-derived xenograft (PDX) model, demonstrated a significantly greater imaging with the noADP construct. The Cox2-ΔE3/ΔADP-NIS vector sustained a significantly earlier, higher intensity, and longer lasting radiotracer [⁹⁹mTcO₄]⁻ uptake compared to other NIS-expressing vectors (FIG. 18). Moreover, the noADP construct was as effective as the ADP+ counterpart in tumor regression (FIG. 19).

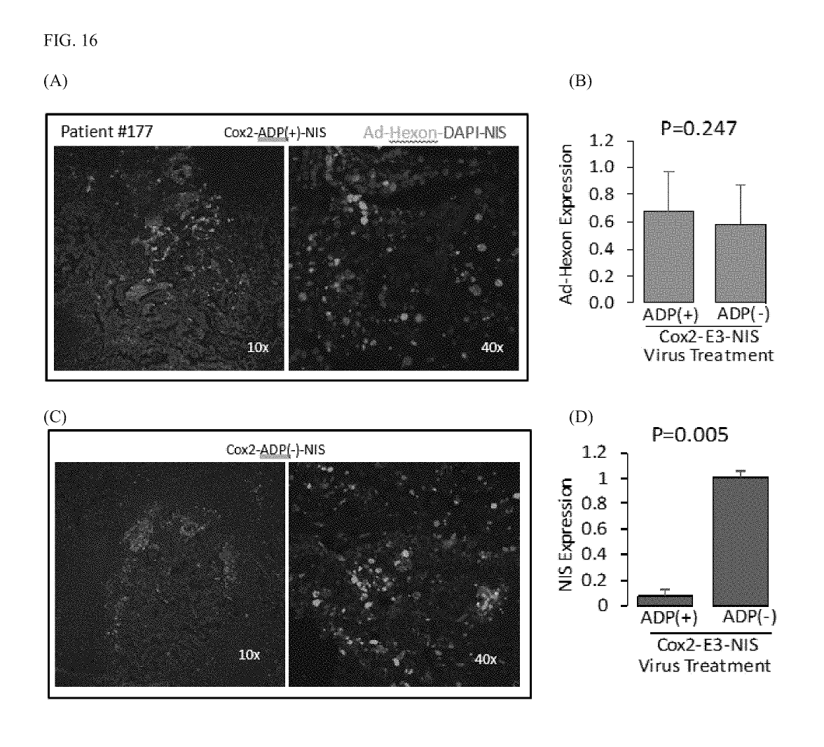

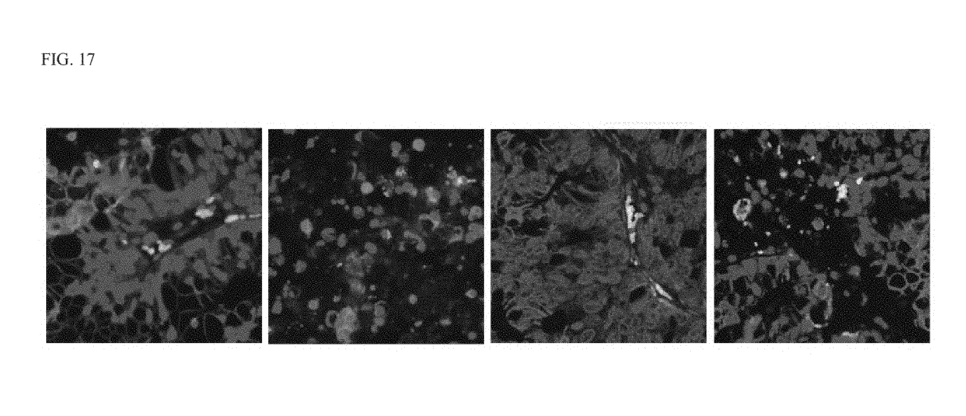

To understand how the noADP construct provides improved and sustained radioisotope uptake in vivo, immunocytochemistry analyses of NIS protein in tumor tissues of virus-treated animals were performed. The Cox2-ΔE3/ΔADP-NIS construct produced more NIS-positive regions in tumors and cell foci than its ADP+ counterpart (FIG. 15). A quantitative assessment of Ad-hexon levels confirm viral infection and spreading were similar between noADP and ADP+. In contrast, noADP vectors expressed significantly more NIS than ADP+ therapy (FIG.16) and this accounted for the observed differences in the radioisotope uptake studies, further supporting its utility as a diagnostic for pancreatic cancer. To assess whether the viral constructs targeted pancreatic adenocarcinoma cells, the intended cell target, human pancreatic cancer xenografts were analyzed for cytokeratin 19, a known cell surface marker of pancreatic adenocarcinoma cells and Ad-hexon as an indication of viral infection. As shown in FIG. 17, human tumors stained positive for Cytokeratin 19 (red) and co-localized with Ad-hexon (green) confirming that our viral constructs targeted pancreatic adenocarcinoma cells.

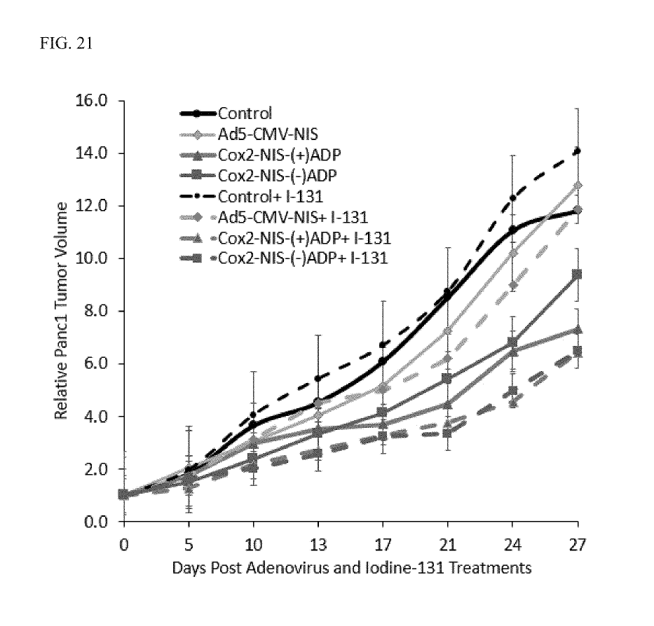

Further clinical evaluation of NIS-expressing adenovirus constructs also demonstrated the ability of oncolytic adenovirus to facilitate therapy with a radioactive iodine [¹³¹I]. The combination of [¹³¹I] with Cox2-ΔE3/ΔADP-NIS significantly reduced tumor progression in a mouse model of human pancreatic cancer (FIG. 21). The detection of I⁻¹³¹with a gamma counter showed a clear trend where ADP(−) retained higher I⁻¹³¹ in tumor tissues than ADP(+) and the AdCMV-NIS positive control. These findings support the clinical applicability of ADP-deleted OAds as more sensitive tools for NIS-based cancer diagnosis and therapy.

Together, these data demonstrate that deleting the Adenovirus Death Protein from the ΔE3-based oncolytic adenovirus improves expression of OAd-delivered transgene at tumor sites. Constructs lacking ADP (noADP) exhibited increased transgene protein expression and radiotracer uptake compare to otherwise identical ADP-positive counterparts. Gene expression from the ΔE3/ΔADP-based vectors was more sustainable and resulted in higher uptake of radioisotope in vitro and in in vivo cancer models, including the clinically relevant patient derived xenograft (PDX) model. In the preceding description and following claims, the term “and/or” means one or all of the listed elements or a combination of any two or more of the listed elements; the terms “comprises,” “comprising,” and variations thereof are to be construed as open ended—i.e., additional elements or steps are optional and may or may not be present; unless otherwise specified, “a,” “an,” “the,” and “at least one” are used interchangeably and mean one or more than one; and the recitations of numerical ranges by endpoints include all numbers subsumed within that range (e.g., 1 to 5 includes 1, 1.5, 2, 2.75, 3, 3.80, 4, 5, etc.).

In the preceding description, particular embodiments may be described in isolation for clarity. Unless otherwise expressly specified that the features of a particular embodiment are incompatible with the features of another embodiment, certain embodiments can include a combination of compatible features described herein in connection with one or more embodiments.

For any method disclosed herein that includes discrete steps, the steps may be conducted in any feasible order. And, as appropriate, any combination of two or more steps may be conducted simultaneously.

The present invention is illustrated by the following examples. It is to be understood that the particular examples, materials, amounts, and procedures are to be interpreted broadly in accordance with the scope and spirit of the invention as set forth herein.

### EXAMPLES

### Example 1

### Vector Design

### The Base: Ad5 ΔE3/ΔADP Backbone

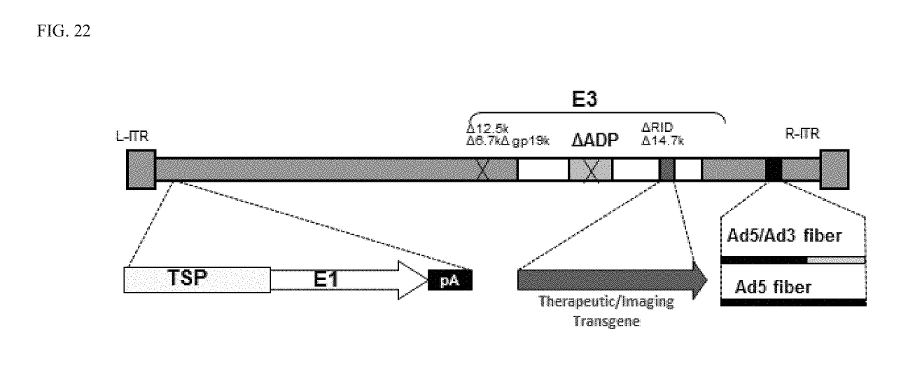

The Ad type 5 vectors containing the therapeutic and/or imaging gene in the E3 region are constructed using the “ΔE3/ΔADP” cloning strategy. Briefly, adenoviral death protein (ADP) and all other nonessential E3 genes (12.5 K, 6.7K, gp19K, RID-α and RID-β, and 14.7K) are deleted (FIG. 22). All viruses were propagated in the 911 cell line, purified by double cesium chloride density gradient ultracentrifugation, and dialyzed against phosphate-buffered saline (PBS) with 10% glycerol. The vectors were titrated by plaque forming assay, and the viral particle (vp) number was measured spectrophotometrically. Viral quality was confirmed by polymerase chain reaction for the presence/absence of ADP, the fiber structure, and the absence of contamination with a mutant replication competent adenovirus as described previously Interchangeable parts:

1. Transgene Inserted into the Adenoviral E3 Region

Deleting the adenoviral E3 genes, as described immediately above, liberates ˜3-5 kb of cloning capacity to accommodate the gene of interest. Expressing the gene of interest from the adenovirus E3 region follows a late profile due to control by the major late promoter and is therefore consistent with the replication cycle. This property is exploited to monitor adenovirus replication and to visualize the target tumors. A ΔE3-based oncolytic adenovirus may be designed to express any suitable transgenes from the adenoviral E3 region. Exemplary transgenes that can be expressed in this manner include, for example, therapeutic genes such as IFNα, INFβ, IL-2, IL-6, IL-7, IL-8, IL-12, GM-CSF, anti-CTLA4 and anti-PD1 antibodies, NIS, or imaging/reporter transgenes such Luc, RFP, GFP, EGFP, mCherry, SSTR, TKsr39, CEA, SEAP, or NIS.

### 2. Promoter Control

The “ΔE3/ΔADP OAds” are controlled with a Tumor Specific Promoter. Vector replication is made tumor-specific by virtue of the tumor-specific promoter control of the adenoviral E1A region. As a consequence, gene expression is restricted to permissive tumors. The Cox-2 promoter is used as a model tumor-specific promoter, providing “tumor on, liver off” profile in pancreatic cancer. However, the promoter may be selected to provide selective expression in appropriate cells for a given disease. Examples include MK, Muc1, PB, PSA, hTERT, GRP, VEGF, SLPI, FLT-1, and caveolin-1 promoters.

### 3. Fiber Modification to Improve “ΔE3/ΔADP” Oncolytic Adenovirus Infectivity

For tumors deficient in the primary adenovirus receptor (coxsackie-adenovirus receptor, CAR—e.g. pancreatic cancer, prostate cancer), the vector can include the Ad5/Ad3-modified fiber. In this fiber, the adenovirus type 5 knob is replaced with the knob taken from Adenovirus type 3, which binds to CD46 and human desmoglein-2 receptor. For tumors that are CAR-positive (i.e., lung cancer), one can use the wild type Ad5 fiber.

### Example 2

### Flow Cytometry

Pancreatic cancer cells were grown in 6 well plates (5×10⁵) until 80% confluent. Confluent cells were left untreated or treated with 1 viral particle per cell for three hours in approximately 500 μl of 5%FBS+ 1%P/S +DMEM cell growth media in a humidified 37° C. chamber. After three hours, media was removed and cells sub-cultured in fresh growth media for another 2-6 days. At 2 days, 4 days, and 6 days post-infection, experimental cells were collected via scraping into tubes on ice. Cells were washed 1× in cold 1× phosphate buffered saline (PBS). Washed cells were fixed in a 4% paraformaldehyde solution, careful to maintain a single cell suspension and washed again in 1× PBS before permeabilization with 0.05% Triton-X100 in PBS solution. Fixed and permeabilized cells were incubated in a 5% Bovine serum albumin (BSA) in 1× PBS solution containing a (1:500) dilution of anti-NIS primary antibody (FPSA, Santa Cruz Biotechnology, Inc., Dallas, Tex.) for approximately one hour on ice. Cells were washed in 1× PBS and re-suspended in 5% BSA containing secondary antibody conjugated to Phycoerythrin (PE) (Red) and incubated for 30 minutes at room temperature. Antibody stained cells were washed and re-suspended in 1% paraformaldehyde solution for one hour. Cells were washed and re-suspended in 1× PBS and analyzed immediately using a FacsCanto (BDBioSciences, San Jose, Calif.).

### Qualitative Evaluation of In Vitro Cytocidal Effect of Oncolytic Adenoviruses

The in vitro cytocidal effect was analyzed via crystal violet staining. One day after 2×10⁵ cells/well were grown in a 12-well plate, viruses were added at strengths of 1 vp/cell. After 6 days, 7 days, or 9 days of cultivation, the cells were fixed with 10% buffered formalin for 10 minutes and stained with 1% crystal violet in 70% ethanol for 20 minutes. Thereafter, the plates were washed three times with tap water and allowed to air dry.

### In Vitro Quantitative Cell Viability Analysis

3,000 cells/well were cultured in 96-well plates and subsequently infected with adenoviral vectors at strengths of 100 vp/cell, 1000 vp/cell, or 10,000 vp/cell. The number of surviving cells was analyzed by a colorimetric method using the Cell Titer Aqueous One Solution Cell Proliferation Assay (Promega, Madison, Wis.) as described by the manufacturer. Absorbance was measured at a wavelength of 490 nm in a FLUOstar Omega spectrophotometer (BMG Labtech, Ortenberg, Germany). The proportion of living cells at each time point was normalized to the number of living uninfected cells.

### Western Blot Analysis

Pancreatic cancer cells were grown in 10 cm² dishes until 80% confluent. Confluent cells were left untreated or infected with 1 viral particle per cell for three hours in 5 ml of normal cell growth media in a humidified 37° C. chamber. After three hours, media was removed and cells sub-cultured in fresh growth media for another two days. At two days post-infection, experimental cells were collected via scraping into tubes on ice. Cells were washed 1× in cold 1× phosphate buffered saline (PBS) and transferred to fresh microfuge tubes. Washed cell pellets were lysed in 100 l of RIPA lysis solution containing fresh PMSF and proteases (Thermo Fisher Scientific, Inc., Waltham, Mass.) on ice. Lysates were fractionated at 14,000× g for 15 minutes in cold, supernatants were collected and placed into fresh chilled microfuge tubes for storage at −70° C. until further use.

Protein concentrations were determined using the DC Protein Estimation Assay and a BSA protein standard curve. Approximately 100 μg of diluted protein samples were diluted 1:4 with sample buffer and heated at 100° C./7 minutes prior to separation using a 7-9% Bis-Tris SDS-PAGE under standard conditions. Proteins were transferred under standard conditions onto PVDF membranes. Membranes were blocked using a 5% non-fat milk in a Tris buffered saline solution containing Tween (TBST) for one hour at room temperature. Blocked membranes were incubated with a (1:500) dilution with anti-NIS primary antibody or (1:200) or anti-actin (FPSA or actin, Santa Cruz Biotechnology, Inc., Dallas, Tex.) overnight with shaking in cold. After incubation, blots were washed in TBST three times for 10 minutes followed by incubation with a secondary antibody conjugated to infrared for 45 minutes shaking at room temperature. Membranes were then imaged using an imaging system (ODYSSEY, Li-Cor Biotechnology, Lincoln, Nebr.).

### MTS Assay

Cells were plated in 96-well plates (Corning, Inc., Corning, N.Y.) in the density of 8×10³ cells/well. Next day, cells were infected with 100 μl of DMEN 5% FBS and 1% penicillin/streptomycin with 1 vp/cell of each virus. Infection was carried out for four hours and media was replaced with appropriate media. DMEM 5% FBS and 1% penicillin/streptomycin with 10 μM or 20 μM 5-FU. Plates were incubated at 37° C. in CO₂ humidified incubator. CellTiter 96 Aqueous One Solution Cell Proliferation Assay MTS (Promega, Madison, Wis.) reagent was added to each well, and plates were incubated for one hour at 37° C. in 5% CO₂ humidified incubator. Plates were read at 490 nm using a plate reader.

Crystal Violet assays

A total of 2×10⁵ cells were plated in a 12-well cell culture plate (Corning, Inc., Corning, N.Y.). Next day, plates were infected with 50 vp/cell or 100 vp/cells of RGDΔE3ADPIFN. After 10 days of cultivation, the cells were fixed with 10% buffered formalin for 10 minutes and stained with 1% crystal violet in 70% ethanol for 20 minutes, followed by washing three times with tap water and air drying.

Immunofluorescence of NIS Pancreatic cancer cells were grown on two 8-well chamber slides (5×10⁴ per well) until 80% confluent. Confluent cells were left untreated or treated with 1 viral particle per cell for three hours in 100 μl of cell growth media in a humidified 37° C. chamber. After three hours, media was removed and cells sub-cultured in fresh growth media for another 3-5 days. At 3 and 5 days post-infection, cells were fixed in chamber slides by first washing with cold 1× PBS. Washed cells were fixed in a 4% paraformaldehyde solution for 20 minutes on ice and then permeabilization with 0.05% Triton-X100 in PBS. Fixed and permeabilized cells were incubated in a 5% Bovine serum albumin (BSA) in 1× PBS solution at a (1:500) dilution with anti-NIS primary antibody or anti-actin (FPSA, Santa Cruz Biotechnology, Inc., Dallas, Tex.) for approximately one hour on ice. Cells were washed in 1× PBS and re-suspended in 5% BSA containing secondary antibody conjugated to phycoerythrin (PE) (Red) and incubated for 30 minutes at room temperature. Antibody stained cells were cover-slipped with DAPI/nuclear stain just prior to image capturing using a fluorescent microscope (40×).

### Immunohistochemistry

Tumor growth assay was conducted by injecting Panc-1 cells subcutaneous in flanks of nude mice. Tumors were injected with either vehicle or 1×10⁹viral particle per tumor.

Pancreatic tumors were embedded at the time of removal into paraffin and later sectioned onto glass slides. Sections were dewaxed in xylenes, washed with ethanol (70%, 50%, 90%, 100%) and rehydrated using distilled water. An additional antigen retrieval step was taken using a universal antigen retrieval solution 1× as instructed (R&D Systems, Inc., Minneapolis, Minn.). Hydrophobic pens were used to draw an outline around tissues. Tissues were permeabilized using a 0.05% Triton-X100 in PBS for five minutes on ice. Sections were washed and incubated in a 5% bovine serum albumin (BSA) in 1× PBS solution for one hour at room temperature to reduce background. Sections were then incubated in a 5% BSA in 1× PBS solution at a (1:500) dilution with anti-NIS primary antibody or anti-hexon-FITC (green) (FPSA, Santa Cruz Biotechnology, Inc., Dallas, Tex.) in cold temperature overnight. Sections were then washed in 1× PBS and incubated in a 5% BSA containing secondary antibody conjugated to phycoerythrin (PE) (Red) and incubated for one hour at room temperature. Antibody stained sections were cover-slipped with DAPI/nuclear stain just prior to image capturing using a fluorescent microscope (40×).

### In Vivo Imaging Analysis and Therapeutic Studies

Human freshly isolated pancreatic cancer tissues or pancreatic cancer cell line Panc-1 was used to establish subcutaneous tumor models in immunodefficient mice. After tumor nodules reached 8-10 mm, a single injection of each virus was given.

### Non-Invasive Imaging of NIS Expressions

At different time points after adenovirus administration, animals received intraperitopneal injections of 0.5 mCi of sodium pertechnetate ([⁹⁹mTcO₄]⁻). To measure radionuclide uptake induced by viral replication, a high-resolution micro-SPECT/CT system was used for planar and fusion images. Planar, real-time images were taken at each time point one hour after isotope injection, with five replicates per viral construct. Tumor size in the region-of-interest was determined by the number of counts present in the tumor, adjusted for background and isotope decay. Tumor growth was measured over the 21 days or up until just prior to its removal.

### In Vivo Combination With the [¹³¹I] Therapy

Three days after adenovirus administration, animals bearing human pancreatic cancer xenografts received intraperitopneal injections of I⁻¹³¹ (3 mCi). Tumor growth was measured over the 27 days for evaluation of the tumor size. Eight days later I⁻¹³¹ treated mice tumors and leg muscle were harvested and relative I⁻¹³¹ uptake was determined using a gamma counter.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present invention. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

Sequence Listing Free Text

SEQ ID NO: 1

| | |
|---|---|
| 1 | CATCATCAAT AATATACCTT ATTTTGGATT GAAGCCAATA TGATAATGAG |
| 51 | GGGGTGGAGT TTGTGACGTG GCGCGGGGCG TGGGAACGGG GCGGGTGACG |
| 101 | TAGTAGTGTG GCGGAAGTGT GATGTTGCAA GTGTGGCGGA ACACATGTAA |
| 151 | GCGACGGATG TGGCAAAAGT GACGTTTTTG GTGTGCGCCG GTGTACACAG |
| 201 | GAAGTGACAA TTTTCGCGCG GTTTTAGGCG GATGTTGTAG TAAATTTGGG |
| 251 | CGTAACCGAG TAAGATTTGG CCATTTTCGC GGGAAAACTG AATAAGAGGA |
| 301 | AGTGAAATCT GAATAATTTT GTGTTACTCA TAGCGCGTAA TATTTGTCTA |
| 351 | GGGCCGCGGG GACTTTGACC GTTTACGTGG AGACTCGCCC AGGTGTTTTT |
| 401 | CTCAGGTGTT TTCCGCGTTC CGGGTCAAAG TTGGCGTTTT ATTATTATAG |
| 451 | TCAGCTGACG TGTAGTGTAT TTATACCCGG TGAGTTCCTC AAGAGGCCAC |
| 501 | TCTTGAGTGC CAGCGAGTAG AGTTTTCTCC TCCGAGCCGC TCCGACACCG |
| 551 | GGACTGAAAA TGAGACATAT TATCTGCCAC GGAGGTGTTA TTACCGAAGA |
| 601 | AATGGCCGCC AGTCTTTTGG ACCAGCTGAT CGAAGAGGTA CTGGCTGATA |
| 651 | ATCTTCCACC TCCTAGCCAT TTTGAACCAC CTACCCTTCA CGAACTGTAT |
| 701 | GATTTAGACG TGACGGCCCC CGAAGATCCC AACGAGGAGG CGGTTTCGCA |
| 751 | GATTTTTCCC GACTCTGTAA TGTTGGCGGT GCAGGAAGGG ATTGACTTAC |
| 801 | TCACTTTTCC GCCGGCGCCC GGTTCTCCGG AGCCGCCTCA CCTTTCCCGG |
| 851 | CAGCCCGAGC AGCCGGAGCA GAGAGCCTTG GGTCCGGTTT CTATGCCAAA |

ClaI

| | |
|---|---|
| 901 | CCTTGTACCG GAGGTGATCG ATCTTACCTG CCACGAGGCT GGCTTTCCAC |
| 951 | CCAGTGACGA CGAGGATGAA GAGGGTGAGG AGTTTGTGTT AGATTATGTG |
| 1001 | GAGCACCCCG GGCACGGTTG CAGGTCTTGT CATTATCACC GGAGGAATAC |
| 1051 | GGGGGACCCA GATATTATGT GTTCGCTTTG CTATATGAGG ACCTGTGGCA |
| 1101 | TGTTTGTCTA CAGTAAGTGA AAATTATGGG CAGTGGGTGA TAGAGTGGTG |
| 1151 | GGTTTGGTGT GGTAATTTTT TTTTTAATTT TTACAGTTTT GTGGTTTAAA |
| 1201 | GAATTTTGTA TTGTGATTTT TTTAAAAGGT CCTGTGTCTG AACCTGAGCC |
| 1251 | TGAGCCCGAG CCAGAACCGG AGCCTGCAAG ACCTACCCGC CGTCCTAAAA |
| 1301 | TGGCGCCTGC TATCCTGAGA CGCCCGACAT CACCTGTGTC TAGAGAATGC |
| 1351 | AATAGTAGTA CGGATAGCTG TGACTCCGGT CCTTCTAACA CACCTCCTGA |
| 1401 | GATACACCCG GTGGTCCCGC TGTGCCCCAT TAAACCAGTT GCCGTGAGAG |
| 1451 | TTGGTGGGCG TCGCCAGGCT GTGGAATGTA TCGAGGACTT GCTTAACGAG |
| 1501 | CCTGGGCAAC CTTTGGACTT GAGCTGTAAA CGCCCCAGGC CATAAGGTGT |
| 1551 | AAACCTGTGA TTGCGTGTGT GGTTAACGCC TTTGTTTGCT GAATGAGTTG |
| 1601 | ATGTAAGTTT AATAAAGGGT GAGATAATGT TTAACTTGCA TGGCGTGTTA |
| 1651 | AATGGGGCGG GGCTTAAAGG GTATATAATG CGCCGTGGGC TAATCTTGGT |
| 1701 | TACATCTGAC CTCATGGAGG CTTGGGAGTG TTTGGAAGAT TTTTCTGCTG |
| 1751 | TGCGTAACTT GCTGGAACAG AGCTCTAACA GTACCTCTTG GTTTTGGAGG |
| 1801 | TTTCTGTGGG GCTCATCCCA GGCAAAGTTA GTCTGCAGAA TTAAGGAGGA |
| 1851 | TTACAAGTGG GAATTTGAAG AGCTTTTGAA ATCCTGTGGT GAGCTGTTTG |
| 1901 | ATTCTTTGAA TCTGGGTCAC CAGGCGCTTT TCCAAGAGAA GGTCATCAAG |
| 1951 | ACTTTGGATT TTTCCACACC GGGGCGCGCT GCGGCTGCTG TTGCTTTTTT |

KpnI

| | |
|---|---|
| 2001 | GAGTTTTATA AAGGATAAAT GGAGCGAAGA AACCCATCTG AGCGGGGGGT |
| 2051 | ACCTGCTGGA TTTTCTGGCC ATGCATCTGT GGAGAGCGGT TGTGAGACAC |
| 2101 | AAGAATCGCC TGCTACTGTT GTCTTCCGTC CGCCCGGCGA TAATACCGAC |
| 2151 | GGAGGAGCAG CAGCAGCAGC AGGAGGAAGC CAGGCGGCGG CGGCAGGAGC |
| 2201 | AGAGCCCATG GAACCCGAGA GCCGGCCTGG ACCCTCGGGA ATGAATGTTG |
| 2251 | TACAGGTGGC TGAACTGTAT CCAGAACTGA GACGCATTTT GACAATTACA |
| 2301 | GAGGATGGGC AGGGGCTAAA GGGGGTAAAG AGGGAGCGGG GGGCTTGTGA |
| 2351 | GGCTACAGAG GAGGCTAGGA ATCTAGCTTT TAGCTTAATG ACCAGACACC |
| 2401 | GTCCTGAGTG TATTACTTTT CAACAGATCA AGGATAATTG CGCTAATGAG |
| 2451 | CTTGATCTGC TGGCGCAGAA GTATTCCATA GAGCAGCTGA CCACTTACTG |
| 2501 | GCTGCAGCCA GGGGATGATT TTGAGGAGGC TATTAGGGTA TATGCAAAGG |
| 2551 | TGGCACTTAG GCCAGATTGC AAGTACAAGA TCAGCAAACT TGTAAATATC |
| 2601 | AGGAATTGTT GCTACATTTC TGGGAACGGG GCCGAGGTGG AGATAGATAC |
| 2651 | GGAGGATAGG GTGGCCTTTA GATGTAGCAT GATAAATATG TGGCCGGGGG |
| 2701 | TGCTTGGCAT GGACGGGGTG GTTATTATGA ATGTAAGGTT TACTGGCCCC |
| 2751 | AATTTTAGCG GTACGGTTTT CCTGGCCAAT ACCAACCTTA TCCTACACGG |

HindIII

| | |
|---|---|
| 2801 | TGTAAGCTTC TATGGGTTTA ACAATACCTG TGTGGAAGCC TGGACCGATG |
| 2851 | TAAGGGTTCG GGGCTGTGCC TTTTACTGCT GCTGGAAGGG GGTGGTGTGT |
| 2901 | CGCCCCAAAA GCAGGGCTTC AATTAAGAAA TGCCTCTTTG AAAGGTGTAC |
| 2951 | CTTGGGTATC CTGTCTGAGG GTAACTCCAG GGTGCGCCAC AATGTGGCCT |
| 3001 | CCGACTGTGG TTGCTTCATG CTAGTGAAAA GCGTGGCTGT GATTAAGCAT |
| 3051 | AACATGGTAT GTGGCAACTG CGAGGACAGG GCCTCTCAGA TGCTGACCTG |
| 3101 | CTCGGACGGC AACTGTCACC TGCTGAAGAC CATTCACGTA GCCAGCCACT |
| 3151 | CTCGCAAGGC CTGGCCAGTG TTTGAGCATA ACATACTGAC CCGCTGTTCC |
| 3201 | TTGCATTTGG GTAACAGGAG GGGGGTGTTC CTACCTTACC AATGCAATTT |
| 3251 | GAGTCACACT AAGATATTGC TTGAGCCCGA GAGCATGTCC AAGGTGAACC |
| 3301 | TGAACGGGGT GTTTGACATG ACCATGAAGA TCTGGAAGGT GCTGAGGTAC |
| 3351 | GATGAGACCC GCACCAGGTG CAGACCCTGC GAGTGTGGCG GTAAACATAT |
| 3401 | TAGGAACCAG CCTGTGATGC TGGATGTGAC CGAGGAGCTG AGGCCCGATC |
| 3451 | ACTTGGTGCT GGCCTGCACC CGCGCTGAGT TTGGCTCTAG CGATGAAGAT |
| 3501 | ACAGATTGAG GTACTGAAAT GTGTGGGCGT GGCTTAAGGG TGGGAAAGAA |
| 3551 | TATATAAGGT GGGGGTCTTA TGTAGTTTTG TATCTGTTTT GCAGCAGCCG |
| 3601 | CCGCCGCCAT GAGCACCAAC TCGTTTGATG GAAGCATTGT GAGCTCATAT |
| 3651 | TTGACAACGC GCATGCCCCC ATGGGCCGGG GTGCGTCAGA ATGTGATGGG |
| 3701 | CTCCAGCATT GATGGTCGCC CCGTCCTGCC CGCAAACTCT ACTACCTTGA |
| 3751 | CCTACGAGAC CGTGTCTGGA ACGCCGTTGG AGACTGCAGC CTCCGCCGCC |
| 3801 | GCTTCAGCCG CTGCAGCCAC CGCCCGCGGG ATTGTGACTG ACTTTGCTTT |
| 3851 | CCTGAGCCCG CTTGCAAGCA GTGCAGCTTC CCGTTCATCC GCCCGCGATG |

MfeI

| | |
|---|---|
| 3901 | ACAAGTTGAC GGCTCTTTTG GCACAATTGG ATTCTTTGAC CCGGGAACTT |
| 3951 | AATGTCGTTT CTCAGCAGCT GTTGGATCTG CGCCAGCAGG TTTCTGCCCT |
| 4001 | GAAGGCTTCC TCCCCTCCCA ATGCGGTTTA AAACATAAAT AAAAAACCAG |
| 4051 | ACTCTGTTTG GATTTGGATC AAGCAAGTGT CTTGCTGTCT TTATTTAGGG |
| 4101 | GTTTTGCGCG CGCGGTAGGC CCGGGACCAG CGGTCTCGGT CGTTGAGGGT |
| 4151 | CCTGTGTATT TTTTCCAGGA CGTGGTAAAG GTGACTCTGG ATGTTCAGAT |
| 4201 | ACATGGGCAT AAGCCCGTCT CTGGGGTGGA GGTAGCACCA CTGCAGAGCT |
| 4251 | TCATGCTGCG GGGTGGTGTT GTAGATGATC CAGTCGTAGC AGGAGCGCTG |
| 4301 | GGCGTGGTGC CTAAAAATGT CTTTCAGTAG CAAGCTGATT GCCAGGGGCA |
| 4351 | GGCCCTTGGT GTAAGTGTTT ACAAAGCGGT TAAGCTGGGA TGGGTGCATA |
| 4401 | CGTGGGGATA TGAGATGCAT CTTGGACTGT ATTTTTAGGT TGGCTATGTT |
| 4451 | CCCAGCCATA TCCCTCCGGG GATTCATGTT GTGCAGAACC ACCAGCACAG |
| 4501 | TGTATCCGGT GCACTTGGGA AATTTGTCAT GTAGCTTAGA AGGAAATGCG |
| 4551 | TGGAAGAACT TGGAGACGCC CTTGTGACCT CCAAGATTTT CCATGCATTC |
| 4601 | GTCCATAATG ATGGCAATGG GCCCACGGGC GGCGGCCTGG GCGAAGATAT |
| 4651 | TTCTGGGATC ACTAACGTCA TAGTTGTGTT CCAGGATGAG ATCGTCATAG |
| 4701 | GCCATTTTTA CAAAGCGCGG GCGGAGGGTG CCAGACTGCG GTATAATGGT |
| 4751 | TCCATCCGGC CCAGGGGCGT AGTTACCCTC ACAGATTTGC ATTTCCCACG |
| 4801 | CTTTGAGTTC AGATGGGGGG ATCATGTCTA CCTGCGGGGC GATGAAGAAA |
| 4851 | ACGGTTTCCG GGGTAGGGGA GATCAGCTGG GAAGAAAGCA GGTTCCTGAG |
| 4901 | CAGCTGCGAC TTACCGCAGC CGGTGGGCCC GTAAATCACA CCTATTACCG |
| 4951 | GGTGCAACTG GTAGTTAAGA GAGCTGCAGC TGCCGTCATC CCTGAGCAGG |
| 5001 | GGGGCCACTT CGTTAAGCAT GTCCCTGACT CGCATGTTTT CCCTGACCAA |
| 5051 | ATCCGCCAGA AGGCGCTCGC CGCCCAGCGA TAGCAGTTCT TGCAAGGAAG |
| 5101 | CAAAGTTTTT CAACGGTTTG AGACCGTCCG CCGTAGGCAT GCTTTTGAGC |
| 5151 | GTTTGACCAA GCAGTTCCAG GCGGTCCCAC AGCTCGGTCA CCTGCTCTAC |
| 5201 | GGCATCTCGA TCCAGCATAT CTCCTCGTTT CGCGGGTTGG GGCGGCTTTC |
| 5251 | GCTGTACGGC AGTAGTCGGT GCTCGTCCAG ACGGGCCAGG GTCATGTCTT |
| 5301 | TCCACGGGCG CAGGGTCCTC GTCAGCGTAG TCTGGGTCAC GGTGAAGGGG |
| 5351 | TGCGCTCCGG GCTGCGCGCT GGCCAGGGTG CGCTTGAGGC TGGTCCTGCT |
| 5401 | GGTGCTGAAG CGCTGCCGGT CTTCGCCCTG CGCGTCGGCC AGGTAGCATT |
| 5451 | TGACCATGGT GTCATAGTCC AGCCCCTCCG CGGCGTGGCC CTTGGCGCGC |
| 5501 | AGCTTGCCCT TGGAGGAGGC GCCGCACGAG GGGCAGTGCA GACTTTTGAG |
| 5551 | GGCGTAGAGC TTGGGCGCGA GAAATACCGA TTCCGGGGAG TAGGCATCCG |
| 5601 | CGCCGCAGGC CCCGCAGACG GTCTCGCATT CCACGAGCCA GGTGAGCTCT |
| 5651 | GGCCGTTCGG GGTCAAAAAC CAGGTTTCCC CCATGCTTTT TGATGCGTTT |
| 5701 | CTTACCTCTG GTTTCCATGA GCCGGTGTCC ACGCTCGGTG ACGAAAAGGC |

XhoI

| | |
|---|---|
| 5751 | TGTCCGTGTC CCCGTATACA GACTTGAGAG GCCTGTCCTC GAGCGGTGTT |
| 5801 | CCGCGGTCCT CCTCGTATAG AAACTCGGAC CACTCTGAGA CAAAGGCTCG |
| 5851 | CGTCCAGGCC AGCACGAAGG AGGCTAAGTG GGAGGGGTAG CGGTCGTTGT |
| 5901 | CCACTAGGGG GTCCACTCGC TCCAGGGTGT GAAGACACAT GTCGCCCTCT |
| 5951 | TCGGCATCAA GGAAGGTGAT TGGTTTGTAG GTGTAGGCCA CGTGACCGGG |
| 6001 | TGTTCCTGAA GGGGGGCTAT AAAAGGGGGT GGGGGCGCGT TCGTCCTCAC |
| 6051 | TCTCTTCCGC ATCGCTGTCT GCGAGGGCCA GCTGTTGGGG TGAGTACTCC |
| 6101 | CTCTGAAAAG CGGGCATGAC TTCTGCGCTA AGATTGTCAG TTTCCAAAAA |
| 6151 | CGAGGAGGAT TTGATATTCA CCTGGCCCGC GGTGATGCCT TTGAGGGTGG |

HindIII

| | |
|---|---|
| 6201 | CCGCATCCAT CTGGTCAGAA AAGACAATCT TTTTGTTGTC AAGCTTGGTG |
| 6251 | GCAAACGACC CGTAGAGGGC GTTGGACAGC AACTTGGCGA TGGAGCGCAG |
| 6301 | GGTTTGGTTT TTGTCGCGAT CGGCGCGCTC CTTGGCCGCG ATGTTTAGCT |
| 6351 | GCACGTATTC GCGCGCAACG CACCGCCATT CGGGAAAGAC GGTGGTGCGC |
| 6401 | TCGTCGGGCA CCAGGTGCAC GCGCCAACCG CGGTTGTGCA GGGTGACAAG |
| 6451 | GTCAACGCTG GTGGCTACCT CTCCGCGTAG GCGCTCGTTG GTCCAGCAGA |

NotI

| | |
|---|---|
| 6501 | GGCGGCCGCC CTTGCGCGAG CAGAATGGCG GTAGGGGGTC TAGCTGCGTC |
| 6551 | TCGTCCGGGG GGTCTGCGTC CACGGTAAAG ACCCCGGGCA GCAGGCGCGC |
| 6601 | GTCGAAGTAG TCTATCTTGC ATCCTTGCAA GTCTAGCGCC TGCTGCCATG |
| 6651 | CGCGGGCGGC AAGCGCGCGC TCGTATGGGT TGAGTGGGGG ACCCCATGGC |
| 6701 | ATGGGGTGGG TGAGCGCGGA GGCGTACATG CCGCAAATGT CGTAAACGTA |
| 6751 | GAGGGGCTCT CTGAGTATTC CAAGATATGT AGGGTAGCAT CTTCCACCGC |
| 6801 | GGATGCTGGC GCGCACGTAA TCGTATAGTT CGTGCGAGGG AGCGAGGAGG |
| 6851 | TCGGGACCGA GGTTGCTACG GGCGGGCTGC TCTGCTCGGA AGACTATCTG |
| 6901 | CCTGAAGATG GCATGTGAGT TGGATGATAT GGTTGGACGC TGGAAGACGT |
| 6951 | TGAAGCTGGC GTCTGTGAGA CCTACCGCGT CACGCACGAA GGAGGCGTAG |
| 7001 | GAGTCGCGCA GCTTGTTGAC CAGCTCGGCG GTGACCTGCA CGTCTAGGGC |
| 7051 | GCAGTAGTCC AGGGTTTCCT TGATGATGTC ATACTTATCC TGTCCCTTTT |
| 7101 | TTTTCCACAG CTCGCGGTTG AGGACAAACT CTTCGCGGTC TTTCCAGTAC |
| 7151 | TCTTGGATCG GAAACCCGTC GGCCTCCGAA CGGTAAGAGC CTAGCATGTA |
| 7201 | GAACTGGTTG ACGGCCTGGT AGGCGCAGCA TCCCTTTTCT ACGGGTAGCG |
| 7251 | CGTATGCCTG CGCGGCCTTC CGGAGCGAGG TGTGGGTGAG CGCAAAGGTG |
| 7301 | TCCCTGACCA TGACTTTGAG GTACTGGTAT TTGAAGTCAG TGTCGTCGCA |
| 7351 | TCCGCCCTGC TCCCAGAGCA AAAAGTCCGT GCGCTTTTTG GAACGCGGAT |
| 7401 | TTGGCAGGGC GAAGGTGACA TCGTTGAAGA GTATCTTTCC CGCGCGAGGC |
| 7451 | ATAAAGTTGC GTGTGATGCG GAAGGGTCCC GGCACCTCGG AACGGTTGTT |
| 7501 | AATTACCTGG GCGGCGAGCA CGATCTCGTC AAAGCCGTTG ATGTTGTGGC |
| 7551 | CCACAATGTA AAGTTCCAAG AAGCGCGGGA TGCCCTTGAT GGAAGGCAAT |
| 7601 | TTTTTAAGTT CCTCGTAGGT GAGCTCTTCA GGGGAGCTGA GCCCGTGCTC |
| 7651 | TGAAAGGGCC CAGTCTGCAA GATGAGGGTT GGAAGCGACG AATGAGCTCC |
| 7701 | ACAGGTCACG GGCCATTAGC ATTTGCAGGT GGTCGCGAAA GGTCCTAAAC |
| 7751 | TGGCGACCTA TGGCCATTTT TTCTGGGGTG ATGCAGTAGA AGGTAAGCGG |
| 7801 | GTCTTGTTCC CAGCGGTCCC ATCCAAGGTT CGCGGCTAGG TCTCGCGCGG |
| 7851 | CAGTCACTAG AGGCTCATCT CCGCCGAACT TCATGACCAG CATGAAGGGC |
| 7901 | ACGAGCTGCT TCCCAAAGGC CCCCATCCAA GTATAGGTCT CTACATCGTA |
| 7951 | GGTGACAAAG AGACGCTCGG TGCGAGGATG CGAGCCGATC GGGAAGAACT |

MfeI

| | |
|---|---|
| 8001 | GGATCTCCCG CCACCAATTG GAGGAGTGGC TATTGATGTG GTGAAAGTAG |
| 8051 | AAGTCCCTGC GACGGGCCGA ACACTCGTGC TGGCTTTTGT AAAAACGTGC |
| 8101 | GCAGTACTGG CAGCGGTGCA CGGGCTGTAC ATCCTGCACG AGGTTGACCT |
| 8151 | GACGACCGCG CACAAGGAAG CAGAGTGGGA ATTTGAGCCC CTCGCCTGGC |
| 8201 | GGGTTTGGCT GGTGGTCTTC TACTTCGGCT GCTTGTCCTT GACCGTCTGG |

XhoI

| | |
|---|---|
| 8251 | CTGCTCGAGG GGAGTTACGG TGGATCGGAC CACCACGCCG CGCGAGCCCA |
| 8301 | AAGTCCAGAT GTCCGCGCGC GGCGGTCGGA GCTTGATGAC AACATCGCGC |
| 8351 | AGATGGGAGC TGTCCATGGT CTGGAGCTCC CGCGGCGTCA GGTCAGGCGG |
| 8401 | GAGCTCCTGC AGGTTTACCT CGCATAGACG GGTCAGGGCG CGGGCTAGAT |
| 8451 | CCAGGTGATA CCTAATTTCC AGGGGCTGGT TGGTGGCGGC GTCGATGGCT |

KpnI

| | |
|---|---|
| 8501 | TGCAAGAGGC CGCATCCCCG CGGCGCGACT ACGGTACCGC GCGGCGGGCG |
| 8551 | GTGGGCCGCG GGGGTGTCCT TGGATGATGC ATCTAAAAGC GGTGACGCGG |
| 8601 | GCGAGCCCCC GGAGGTAGGG GGGGCTCCGG ACCCGCCGGG AGAGGGGGCA |
| 8651 | GGGGCACGTC GGCGCCGCGC GCGGGCAGGA GCTGGTGCTG CGCGCGTAGG |
| 8701 | TTGCTGGCGA ACGCGACGAC GCGGCGGTTG ATCTCCTGAA TCTGGCGCCT |
| 8751 | CTGCGTGAAG ACGACGGGCC CGGTGAGCTT GAGCCTGAAA GAGAGTTCGA |
| 8801 | CAGAATCAAT TTCGGTGTCG TTGACGGCGG CCTGGCGCAA AATCTCCTGC |
| 8851 | ACGTCTCCTG AGTTGTCTTG ATAGGCGATC TCGGCCATGA ACTGCTCGAT |
| 8901 | CTCTTCCTCC TGGAGATCTC CGCGTCCGGC TCGCTCCACG GTGGCGGCGA |
| 8951 | GGTCGTTGGA AATGCGGGCC ATGAGCTGCG AGAAGGCGTT GAGGCCTCCC |
| 9001 | TCGTTCCAGA CGCGGCTGTA GACCACGCCC CCTTCGGCAT CGCGGGCGCG |
| 9051 | CATGACCACC TGCGCGAGAT TGAGCTCCAC GTGCCGGGCG AAGACGGCGT |
| 9101 | AGTTTCGCAG GCGCTGAAAG AGGTAGTTGA GGGTGGTGGC GGTGTGTTCT |

EcoRV

| | |
|---|---|
| 9151 | GCCACGAAGA AGTACATAAC CCAGCGTCGC AACGTGGATT CGTTGATATC |
| 9201 | CCCCAAGGCC TCAAGGCGCT CCATGGCCTC GTAGAAGTCC ACGGCGAAGT |
| 9151 | TGAAAAACTG GGAGTTGCGC GCCGACACGG TTAACTCCTC CTCCAGAAGA |
| 9301 | CGGATGAGCT CGGCGACAGT GTCGCGCACC TCGCGCTCAA AGGCTACAGG |
| 9351 | GGCCTCTTCT TCTTCTTCAA TCTCCTCTTC CATAAGGGCC TCCCCTTCTT |
| 9401 | CTTCTTCTGG CGGCGGTGGG GGAGGGGGGA CACGGCGGCG ACGACGGCGC |

SalI

| | |
|---|---|
| 9451 | ACCGGGAGGC GGTCGACAAA GCGCTCGATC ATCTCCCCGC GGCGACGGCG |
| 9501 | CATGGTCTCG GTGACGGCGC GGCCGTTCTC GCGGGGGCGC AGTTGGAAGA |
| 9551 | CGCCGCCCGT CATGTCCCGG TTATGGGTTG GCGGGGGGCT GCCATGCGGC |

MfeI

| | |
|---|---|
| 9601 | AGGGATACGG CGCTAACGAT GCATCTCAAC AATTGTTGTG TAGGTACTCC |

XhoI

| | |
|---|---|
| 9651 | GCCGCCGAGG GACCTGAGCG AGTCCGCATC GACCGGATCG GAAAACCTCT |
| 9701 | CGAGAAAGGC GTCTAACCAG TCACAGTCGC AAGGTAGGCT GAGCACCGTG |
| 9751 | GCGGGCGGCA GCGGGCGGCG GTCGGGGTTG TTTCTGGCGG AGGTGCTGCT |

SalI

| | |
|---|---|
| 9801 | GATGATGTAA TTAAAGTAGG CGGTCTTGAG ACGGCGGATG GTCGACAGAA |
| 9851 | GCACCATGTC CTTGGGTCCG GCCTGCTGAA TGCGCAGGCG GTCGGCCATG |
| 9901 | CCCCAGGCTT CGTTTTGACA TCGGCGCAGG TCTTTGTAGT AGTCTTGCAT |
| 9951 | GAGCCTTTCT ACCGGCACTT CTTCTTCTCC TTCCTCTTGT CCTGCATCTC |
| 10001 | TTGCATCTAT CGCTGCGGCG GCGGCGGAGT TTGGCCGTAG GTGGCGCCCT |
| 10051 | CTTCCTCCCA TGCGTGTGAC CCCGAAGCCC CTCATCGGCT GAAGCAGGGC |
| 10101 | TAGGTCGGCG ACAACGCGCT CGGCTAATAT GGCCTGCTGC ACCTGCGTGA |
| 10151 | GGGTAGACTG GAAGTCATCC ATGTCCACAA AGCGGTGGTA TGCGCCCGTG |
| 10202 | TTGATGGTGT AAGTGCAGTT GGCCATAACG GACCAGTTAA CGGTCTGGTG |

XhoI

| | |
|---|---|
| 10251 | ACCCGGCTGC GAGAGCTCGG TGTACCTGAG ACGCGAGTAA GCCCTCGAGT |
| 10301 | CAAATACGTA GTCGTTGCAA GTCCGCACCA GGTACTGGTA TCCCACCAAA |
| 10351 | AAGTGCGGCG GCGGCTGGCG GTAGAGGGGC CAGCGTAGGG TGGCCGGGGC |

EcoRV

| | |
|---|---|
| 10401 | TCCGGGGGCG AGATCTTCCA ACATAAGGCG ATGATATCCG TAGATGTACC |
| 10451 | TGGACATCCA GGTGATGCCG GCGGCGGTGG TGGAGGCGCG CGGAAAGTCG |
| 10501 | CGGACGCGGT TCCAGATGTT GCGCAGCGGC AAAAAGTGCT CCATGGTCGG |
| 10551 | GACGCTCTGG CCGGTCAGGC GCGCGCAATC GTTGACGCTC TAGACCGTGC |
| 10601 | AAAAGGAGAG CCTGTAAGCG GGCACTCTTC CGTGGTCTGG TGGATAAATT |
| 10651 | CGCAAGGGTA TCATGGCGGA CGACCGGGGT TCGAGCCCCG TATCCGGCCG |
| 10701 | TCCGCCGTGA TCCATGCGGT TACCGCCCGC GTGTCGAACC CAGGTGTGCG |
| 10751 | ACGTCAGACA ACGGGGGAGT GCTCCTTTTG GCTTCCTTCC AGGCGCGGCG |

NheI

| | |
|---|---|
| 10801 | GCTGCTGCGC TAGCTTTTTT GGCCACTGGC CGCGCGCAGC GTAAGCGGTT |
| 10851 | AGGCTGGAAA GCGAAAGCAT TAAGTGGCTC GCTCCCTGTA GCCGGAGGGT |
| 10901 | TATTTTCCAA GGGTTGAGTC GCGGGACCCC CGGTTCGAGT CTCGGACCGG |
| 10951 | CCGGACTGCG GCGAACGGGG GTTTGCCTCC CCGTCATGCA AGACCCCGCT |
| 11001 | TGCAAATTCC TCCGGAAACA GGGACGAGCC CCTTTTTTGC TTTTCCCAGA |

M Frame 1

| | |
|---|---|
| 11051 | TGCATCCGGT GCTGCGGCAG ATGCGCCCCC CTCCTCAGCA GCGGCAAGAG |
| 11101 | CAAGAGCAGC GGCAGACATG CAGGGCACCC TCCCCTCCTC CTACCGCGTC |
| 11151 | AGGAGGGGCG ACATCCGCGG TTGACGCGGC AGCAGATGGT GATTACGAAC |
| 11201 | CCCCGCGGCG CCGGGCCCGG CACTACCTGG ACTTGGAGGA GGGCGAGGGC |

KpnI

| | |
|---|---|
| 11251 | CTGGCGCGGC TAGGAGCGCC CTCTCCTGAG CGGTACCCAA GGGTGCAGCT |

MluI

| | |
|---|---|
| 11301 | GAAGCGTGAT ACGCGTGAGG CGTACGTGCC GCGGCAGAAC CTGTTTCGCG |
| 11351 | ACCGCGAGGG AGAGGAGCCC GAGGAGATGC GGGATCGAAA GTTCCACGCA |
| 11401 | GGGCGCGAGC TGCGGCATGG CCTGAATCGC GAGCGGTTGC TGCGCGAGGA |
| 11451 | GGACTTTGAG CCCGACGCGC GAACCGGGAT TAGTCCCGCG CGCGCACACG |

NotI

| | |
|---|---|
| 11501 | TGGCGGCCGC CGACCTGGTA ACCGCATACG AGCAGACGGT GAACCAGGAG |

HindIII

| | |
|---|---|
| 11551 | ATTAACTTTC AAAAAAGCTT TAACAACCAC GTGCGTACGC TTGTGGCGCG |
| 11601 | CGAGGAGGTG GCTATAGGAC TGATGCATCT GTGGGACTTT GTAAGCGCGC |
| 11651 | TGGAGCAAAA CCCAAATAGC AAGCCGCTCA TGGCGCAGCT GTTCCTTATA |
| 11701 | GTGCAGCACA GCAGGGACAA CGAGGCATTC AGGGATGCGC TGCTAAACAT |
| 11751 | AGTAGAGCCC GAGGGCCGCT GGCTGCTCGA TTTGATAAAC ATCCTGCAGA |
| 11801 | GCATAGTGGT GCAGGAGCGC AGCTTGAGCC TGGCTGACAA GGTGGCCGCC |
| 11851 | ATCAACTATT CCATGCTTAG CCTGGGCAAG TTTTACGCCC GCAAGATATA |
| 11901 | CCATACCCCT TACGTTCCCA TAGACAAGGA GGTAAAGATC GAGGGGTTCT |
| 11951 | ACATGCGCAT GGCGCTGAAG GTGCTTACCT TGAGCGACGA CCTGGGCGTT |
| 12001 | TATCGCAACG AGCGCATCCA CAAGGCCGTG AGCGTGAGCC GGCGGCGCGA |
| 12051 | GCTCAGCGAC CGCGAGCTGA TGCACAGCCT GCAAAGGGCC CTGGCTGGCA |
| 12101 | CGGGCAGCGG CGATAGAGAG GCCGAGTCCT ACTTTGACGC GGGCGCTGAC |
| 12151 | CTGCGCTGGG CCCCAAGCCG ACGCGCCCTG GAGGCAGCTG GGGCCGGACC |
| 12201 | TGGGCTGGCG GTGGCACCCG CGCGCGCTGG CAACGTCGGC GGCGTGGAGG |
| 12251 | AATATGACGA GGACGATGAG TACGAGCCAG AGGACGGCGA GTACTAAGCG |
| 12301 | GTGATGTTTC TGATCAGATG ATGCAAGACG CAACGGACCC GGCGGTGCGG |
| 12351 | GCGGCGCTGC AGAGCCAGCC GTCCGGCCTT AACTCCACGG ACGACTGGCG |

MluI

| | |
|---|---|
| 12401 | CCAGGTCATG GACCGCATCA TGTCGCTGAC TGCGCGCAAT CCTGACGCGT |
| 12451 | TCCGGCAGCA GCCGCAGGCC AACCGGCTCT CCGCAATTCT GGAAGCGGTG |
| 12501 | GTCCCGGCGC GCGCAAACCC CACGCACGAG AAGGTGCTGG CGATCGTAAA |
| 12551 | CGCGCTGGCC GAAAACAGGG CCATCCGGCC CGACGAGGCC GGCCTGGTCT |
| 12601 | ACGACGCGCT GCTTCAGCGC GTGGCTCGTT ACAACAGCGG CAACGTGCAG |
| 12651 | ACCAACCTGG ACCGGCTGGT GGGGGATGTG CGCGAGGCCG TGGCGCAGCG |
| 12701 | TGAGCGCGCG CAGCAGCAGG GCAACCTGGG CTCCATGGTT GCACTAAACG |
| 12751 | CCTTCCTGAG TACACAGCCC GCCAACGTGC CGCGGGGACA GGAGGACTAC |
| 12801 | ACCAACTTTG TGAGCGCACT GCGGCTAATG GTGACTGAGA CACCGCAAAG |
| 12851 | TGAGGTGTAC CAGTCTGGGC CAGACTATTT TTTCCAGACC AGTAGACAAG |
| 12901 | GCCTGCAGAC CGTAAACCTG AGCCAGGCTT TCAAAAACTT GCAGGGGCTG |
| 12951 | TGGGGGGTGC GGGCTCCCAC AGGCGACCGC GCGACCGTGT CTAGCTTGCT |
| 13001 | GACGCCCAAC TCGCGCCTGT TGCTGCTGCT AATAGCGCCC TTCACGGACA |
| 13051 | GTGGCAGCGT GTCCCGGGAC ACATACCTAG GTCACTTGCT GACACTGTAC |
| 13101 | CGCGAGGCCA TAGGTCAGGC GCATGTGGAC GAGCATACTT TCCAGGAGAT |
| 13151 | TACAAGTGTC AGCCGCGCGC TGGGGCAGGA GGACACGGGC AGCCTGGAGG |
| 13201 | CAACCCTAAA CTACCTGCTG ACCAACCGGC GGCAGAAGAT CCCCTCGTTG |

PmeI

| | |
|---|---|
| 13251 | CACAGTTTAA ACAGCGAGGA GGAGCGCATT TTGCGCTACG TGCAGCAGAG |
| 13301 | CGTGAGCCTT AACCTGATGC GCGACGGGGT AACGCCCAGC GTGGCGCTGG |
| 13351 | ACATGACCGC GCGCAACATG GAACCGGGCA TGTATGCCTC AAACCGGCCG |

NotI

| | |
|---|---|
| 13401 | TTTATCAACC GCCTAATGGA CTACTTGCAT CGCGCGGCCG CCGTGAACCC |
| 13451 | CGAGTATTTC ACCAATGCCA TCTTGAACCC GCACTGGCTA CCGCCCCCTG |
| 13501 | GTTTCTACAC CGGGGGATTC GAGGTGCCCG AGGGTAACGA TGGATTCCTC |
| 13551 | TGGGACGACA TAGACGACAG CGTGTTTTCC CCGCAACCGC AGACCCTGCT |

HindIII

| | |
|---|---|
| 13601 | AGAGTTGCAA CAGCGCGAGC AGGCAGAGGC GGCGCTGCGA AAGGAAAGCT |
| 13651 | TCCGCAGGCC AAGCAGCTTG TCCGATCTAG GCGCTGCGGC CCCGCGGTCA |

HindIII

| | |
|---|---|
| 13701 | GATGCTAGTA GCCCATTTCC AAGCTTGATA GGGTCTCTTA CCAGCACTCG |
| 13751 | CACCACCCGC CCGCGCCTGC TGGGCGAGGA GGAGTACCTA AACAACTCGC |
| 13801 | TGCTGCAGCC GCAGCGCGAA AAAAACCTGC CTCCGGCATT TCCCAACAAC |
| 13851 | GGGATAGAGA GCCTAGTGGA CAAGATGAGT AGATGGAAGA CGTACGCGCA |
| 13901 | GGAGCACAGG GACGTGCCAG GCCCGCGCCC GCCCACCCGT CGTCAAAGGC |
| 13951 | ACGACCGTCA GCGGGGTCTG GTGTGGGAGG ACGATGACTC GGCAGACGAC |
| 14001 | AGCAGCGTCC TGGATTTGGG AGGGAGTGGC AACCCGTTTG CGCACCTTCG |
| 14051 | CCCCAGGCTG GGGAGAATGT TTTAAAAAAA AAAAAGCATG ATGCAAAATA |
| 14101 | AAAAACTCAC CAAGGCCATG GCACCGAGCG TTGGTTTTCT TGTATTCCCC |
| 14151 | TTAGTATGCG GCGCGCGGCG ATGTATGAGG AAGGTCCTCC TCCCTCCTAC |
| 14201 | GAGAGTGTGG TGAGCGCGGC GCCAGTGGCG GCGGCGCTGG GTTCTCCCTT |

KpnI

| | |
|---|---|
| 14251 | CGATGCTCCC CTGGACCCGC CGTTTGTGCC TCCGCGGTAC CTGCGGCCTA |
| 14301 | CCGGGGGGAG AAACAGCATC CGTTACTCTG AGTTGGCACC CCTATTCGAC |
| 14351 | ACCACCCGTG TGTACCTGGT GGACAACAAG TCAACGGATG TGGCATCCCT |
| 14401 | GAACTACCAG AACGACCACA GCAACTTTCT GACCACGGTC ATTCAAAACA |

AgeI

| | |
|---|---|
| 14451 | ATGACTACAG CCCGGGGGAG GCAAGCACAC AGACCATCAA TCTTGACGAC |
| 14501 | CGGTCGCACT GGGGCGGCGA CCTGAAAACC ATCCTGCATA CCAACATGCC |
| 14551 | AAATGTGAAC GAGTTCATGT TTACCAATAA GTTTAAGGCG CGGGTGATGG |
| 14601 | TGTCGCGCTT GCCTACTAAG GACAATCAGG TGGAGCTGAA ATACGAGTGG |
| 14651 | GTGGAGTTCA CGCTGCCCGA GGGCAACTAC TCCGAGACCA TGACCATAGA |
| 14701 | CCTTATGAAC AACGCGATCG TGGAGCACTA CTTGAAAGTG GGCAGACAGA |
| 14751 | ACGGGGTTCT GGAAAGCGAC ATCGGGGTAA AGTTTGACAC CCGCAACTTC |
| 14801 | AGACTGGGGT TTGACCCCGT CACTGGTCTT GTCATGCCTG GGGTATATAC |
| 14851 | AAACGAAGCC TTCCATCCAG ACATCATTTT GCTGCCAGGA TGCGGGGTGG |
| 14901 | ACTTCACCCA CAGCCGCCTG AGCAACTTGT TGGGCATCCG CAAGCGGCAA |
| 14951 | CCCTTCCAGG AGGGCTTTAG GATCACCTAC GATGATCTGG AGGGTGGTAA |
| 15001 | CATTCCCGCA CTGTTGGATG TGGACGCCTA CCAGGCGAGC TTGAAAGATG |
| 15051 | ACACCGAACA GGGCGGGGGT GGCGCAGGCG GCAGCAACAG CAGTGGCAGC |
| 15101 | GGCGCGGAAG AGAACTCCAA CGCGGCAGCC GCGGCAATGC AGCCGGTGGA |
| 15151 | GGACATGAAC GATCATGCCA TTCGCGGCGA CACCTTTGCC ACACGGGCTG |
| 15201 | AGGAGAAGCG CGCTGAGGCC GAAGCAGCGG CCGAAGCTGC CGCCCCCGCT |

AgeI

| | |
|---|---|
| 15251 | GCGCAACCCG AGGTCGAGAA GCCTCAGAAG AAACCGGTGA TCAAACCCCT |
| 15301 | GACAGAGGAC AGCAAGAAAC GCAGTTACAA CCTAATAAGC AATGACAGCA |

KpnI

| | |
|---|---|
| 15351 | CCTTCACCCA GTACCGCAGC TGGTACCTTG CATACAACTA CGGCGACCCT |
| 15401 | CAGACCGGAA TCCGCTCATG GACCCTGCTT TGCACTCCTG ACGTAACCTG |
| 15451 | CGGCTCGGAG CAGGTCTACT GGTCGTTGCC AGACATGATG CAAGACCCCG |
| 15501 | TGACCTTCCG CTCCACGCGC CAGATCAGCA ACTTTCCGGT GGTGGGCGCC |
| 15551 | GAGCTGTTGC CCGTGCACTC CAAGAGCTTC TACAACGACC AGGCCGTCTA |
| 15601 | CTCCCAACTC ATCCGCCAGT TTACCTCTCT GACCCACGTG TTCAATCGCT |
| 15651 | TTCCCGAGAA CCAGATTTTG GCGCGCCCGC CAGCCCCCAC CATCACCACC |
| 15701 | GTCAGTGAAA ACGTTCCTGC TCTCACAGAT CACGGGACGC TACCGCTGCG |
| 15751 | CAACAGCATC GGAGGAGTCC AGCGAGTGAC CATTACTGAC GCCAGACGCC |
| 15801 | GCACCTGCCC CTACGTTTAC AAGGCCCTGG GCATAGTCTC GCCGCGCGTC |
| 15851 | CTATCGAGCC GCACTTTTTG AGCAAGCATG TCCATCCTTA TATCGCCCAG |
| 15901 | CAATAACACA GGCTGGGGCC TGCGCTTCCC AAGCAAGATG TTTGGCGGGG |
| 15951 | CCAAGAAGCG CTCCGACCAA CACCCAGTGC GCGTGCGCGG GCACTACCGC |

NotI

| | |
|---|---|
| 16001 | GCGCCCTGGG GCGCGCACAA ACGCGGCCGC ACTGGGCGCA CCACCGTCGA |
| 16051 | TGACGCCATC GACGCGGTGG TGGAGGAGGC GCGCAACTAC ACGCCCACGC |
| 16101 | CGCCACCAGT GTCCACAGTG GACGCGGCCA TTCAGACCGT GGTGCGCGGA |
| 16151 | GCCCGGCGCT ATGCTAAAAT GAAGAGACGG CGGAGGCGCG TAGCACGTCG |
| 16201 | CCACCGCCGC CGACCCGGCA CTGCCGCCCA ACGCGCGGCG GCGGCCCTGC |
| 16251 | TTAACCGCGC ACGTCGCACC GGCCGACGGG CGGCCATGCG GGCCGCTCGA |

NotI

| | |
|---|---|
| 16301 | AGGCTGGCCG CGGGTATTGT CACTGTGCCC CCCAGGTCCA GGCGACGAGC |
| 16351 | GGCCGCCGCA GCAGCCGCGG CCATTAGTGC TATGACTCAG GGTCGCAGGG |
| 16401 | GCAACGTGTA TTGGGTGCGC GACTCGGTTA GCGGCCTGCG CGTGCCCGTG |
| 16451 | CGCACCCGCC CCCCGCGCAA CTAGATTGCA AGAAAAAACT ACTTAGACTC |
| 16501 | GTACTGTTGT ATGTATCCAG CGGCGGCGGC GCGCAACGAA GCTATGTCCA |
| 16551 | AGCGCAAAAT CAAAGAAGAG ATGCTCCAGG TCATCGCGCC GGAGATCTAT |
| 16601 | GGCCCCCCGA AGAAGGAAGA GCAGGATTAC AAGCCCCGAA AGCTAAAGCG |
| 16651 | GGTCAAAAAG AAAAAGAAAG ATGATGATGA TGAACTTGAC GACGAGGTGG |

MluI
SalI

| | |
|---|---|
| 16701 | AACTGCTGCA CGCTACCGCG CCCAGGCGAC GGGTACAGTG GAAAGGTCGA |
| 16751 | CGCGTAAAAC GTGTTTTGCG ACCCGGCACC ACCGTAGTCT TTACGCCCGG |
| 16801 | TGAGCGCTCC ACCCGCACCT ACAAGCGCGT GTATGATGAG GTGTACGGCG |
| 16851 | ACGAGGACCT GCTTGAGCAG GCCAACGAGC GCCTCGGGGA GTTTGCCTAC |
| 16901 | GGAAAGCGGC ATAAGGACAT GCTGGCGTTG CCGCTGGACG AGGGCAACCC |
| 16951 | AACACCTAGC CTAAAGCCCG TAACACTGCA GCAGGTGCTG CCCGCGCTTG |
| 17001 | CACCGTCCGA AGAAAAGCGC GGCCTAAAGC GCGAGTCTGG TGACTTGGCA |

KpnI

| | |
|---|---|
| 17051 | CCCACCGTGC AGCTGATGGT ACCCAAGCGC CAGCGACTGG AAGATGTCTT |
| 17101 | GGAAAAAATG ACCGTGGAAC CTGGGCTGGA GCCCGAGGTC CGCGTGCGGC |
| 17151 | CAATCAAGCA GGTGGCGCCG GGACTGGGCG TGCAGACCGT GGACGTTCAG |
| 17201 | ATACCCACTA CCAGTAGCAC CAGTATTGCC ACCGCCACAG AGGGCATGGA |
| 17251 | GACACAAACG TCCCCGGTTG CCTCAGCGGT GGCGGATGCC GCGGTGCAGG |

NotI

| | |
|---|---|
| 17301 | CGGTCGCTGC GGCCGCGTCC AAGACCTCTA CGGAGGTGCA AACGGACCCG |
| 17351 | TGGATGTTTC GCGTTTCAGC CCCCCGGCGC CCGCGCGGTT CGAGGAAGTA |
| 17401 | CGGCGCCGCC AGCGCGCTAC TGCCCGAATA TGCCCTACAT CCTTCCATTG |
| 17451 | CGCCTACCCC CGGCTATCGT GGCTACACCT ACCGCCCCAG AAGACGAGCA |
| 17501 | ACTACCCGAC GCCGAACCAC CACTGGAACC CGCCGCCGCC GTCGCCGTCG |
| 17551 | CCAGCCCGTG CTGGCCCCGA TTTCCGTGCG CAGGGTGGCT CGCGAAGGAG |
| 17601 | GCAGGACCCT GGTGCTGCCA ACAGCGCGCT ACCACCCCAG CATCGTTTAA |
| 17651 | AAGCCGGTCT TTGTGGTTCT TGCAGATATG GCCCTCACCT GCCGCCTCCG |
| 17701 | TTTCCCGGTG CCGGGATTCC GAGGAAGAAT GCACCGTAGG AGGGGCATGG |
| 17751 | CCGGCCACGG CCTGACGGGC GGCATGCGTC GTGCGCACCA CCGGCGGCGG |
| 17801 | CGCGCGTCGC ACCGTCGCAT GCGCGGCGGT ATCCTGCCCC TCCTTATTCC |
| 17851 | ACTGATCGCC GCGGCGATTG GCGCCGTGCC CGGAATTGCA TCCGTGGCCT |
| 17901 | TGCAGGCGCA GAGACACTGA TTAAAAACAA GTTGCATGTG GAAAAATCAA |
| 17951 | AATAAAAAGT CTGGACTCTC ACGCTCGCTT GGTCCTGTAA CTATTTTGTA |
| 18001 | GAATGGAAGA CATCAACTTT GCGTCTCTGG CCCCGCGACA CGGCTCGCGC |

EcoRV

| | |
|---|---|
| 18051 | CCGTTCATGG GAAACTGGCA AGATATCGGC ACCAGCAATA TGAGCGGTGG |
| 18101 | CGCCTTCAGC TGGGGCTCGC TGTGGAGCGG CATTAAAAAT TTCGGTTCCA |
| 18151 | CCGTTAAGAA CTATGGCAGC AAGGCCTGGA ACAGCAGCAC AGGCCAGATG |
| 18201 | CTGAGGGATA AGTTGAAAGA GCAAAATTTC CAACAAAAGG TGGTAGATGG |
| 18251 | CCTGGCCTCT GGCATTAGCG GGGTGGTGGA CCTGGCCAAC CAGGCAGTGC |

HindIII

| | |
|---|---|
| 18301 | AAAATAAGAT TAACAGTAAG CTTGATCCCC GCCCTCCCGT AGAGGAGCCT |
| 18351 | CCACCGGCCG TGGAGACAGT GTCTCCAGAG GGGCGTGGCG AAAAGCGTCC |
| 18401 | GCGCCCCGAC AGGGAAGAAA CTCTGGTGAC GCAAATAGAC GAGCCTCCCT |
| 18451 | CGTACGAGGA GGCACTAAAG CAAGGCCTGC CCACCACCCG TCCCATCGCG |
| 18501 | CCCATGGCTA CCGGAGTGCT GGGCCAGCAC ACACCCGTAA CGCTGGACCT |
| 18551 | GCCTCCCCCC GCCGACACCC AGCAGAAACC TGTGCTGCCA GGCCCGACCG |
| 18601 | CCGTTGTTGT AACCCGTCCT AGCCGCGCGT CCCTGCGCCG CGCCGCCAGC |
| 18651 | GGTCCGCGAT CGTTGCGGCC CGTAGCCAGT GGCAACTGGC AAAGCACACT |
| 18701 | GAACAGCATC GTGGGTCTGG GGGTGCAATC CCTGAAGCGC CGACGATGCT |
| 18751 | TCTGAATAGC TAACGTGTCG TATGTGTGTC ATGTATGCGT CCATGTCGCC |
| 18801 | GCCAGAGGAG CTGCTGAGCC GCCGCGCGCC CGCTTTCCAA GATGGCTACC |
| 18851 | CCTTCGATGA TGCCGCAGTG GTCTTACATG CACATCTCGG GCCAGGACGC |
| 18901 | CTCGGAGTAC CTGAGCCCCG GGCTGGTGCA GTTTGCCCGC GCCACCGAGA |
| 18951 | CGTACTTCAG CCTGAATAAC AAGTTTAGAA ACCCCACGGT GGCGCCTACG |

AgeI

| | |
|---|---|
| 19001 | CACGACGTGA CCACAGACCG GTCCCAGCGT TTGACGCTGC GGTTCATCCC |
| 19051 | TGTGGACCGT GAGGATACTG CGTACTCGTA CAAGGCGCGG TTCACCCTAG |
| 19101 | CTGTGGGTGA TAACCGTGTG CTGGACATGG CTTCCACGTA CTTTGACATC |
| 19151 | CGCGGCGTGC TGGACAGGGG CCCTACTTTT AAGCCCTACT CTGGCACTGC |
| 19201 | CTACAACGCC CTGGCTCCCA AGGGTGCCCC AAATCCTTGC GAATGGGATG |
| 19251 | AAGCTGCTAC TGCTCTTGAA ATAAACCTAG AAGAAGAGGA CGATGACAAC |
| 19301 | GAAGACGAAG TAGACGAGCA AGCTGAGCAG CAAAAAACTC ACGTATTTGG |
| 19351 | GCAGGCGCCT TATTCTGGTA TAAATATTAC AAAGGAGGGT ATTCAAATAG |
| 19401 | GTGTCGAAGG TCAAACACCT AAATATGCCG ATAAAACATT TCAACCTGAA |
| 19451 | CCTCAAATAG GAGAATCTCA GTGGTACGAA ACTGAAATTA ATCATGCAGC |

NdeI

| | |
|---|---|
| 19501 | TGGGAGAGTC CTTAAAAAGA CTACCCCAAT GAAACCATGT TACGGTTCAT |
| 19551 | ATGCAAAACC CACAAATGAA AATGGAGGGC AAGGCATTCT TGTAAAGCAA |
| 19601 | CAAAATGGAA AGCTAGAAAG TCAAGTGGAA ATGCAATTTT TCTCAACTAC |
| 19651 | TGAGGCGACC GCAGGCAATG GTGATAACTT GACTCCTAAA GTGGTATTGT |
| 19701 | ACAGTGAAGA TGTAGATATA GAAACCCCAG ACACTCATAT TTCTTACATG |
| 19751 | CCCACTATTA AGGAAGGTAA CTCACGAGAA CTAATGGGCC AACAATCTAT |
| 19801 | GCCCAACAGG CCTAATTACA TTGCTTTTAG GGACAATTTT ATTGGTCTAA |
| 19851 | TGTATTACAA CAGCACGGGT AATATGGGTG TTCTGGCGGG CCAAGCATCG |
| 19901 | CAGTTGAATG CTGTTGTAGA TTTGCAAGAC AGAAACACAG AGCTTTCATA |
| 19951 | CCAGCTTTTG CTTGATTCCA TTGGTGATAG AACCAGGTAC TTTTCTATGT |
| 20001 | GGAATCAGGC TGTTGACAGC TATGATCCAG ATGTTAGAAT TATTGAAAAT |
| 20051 | CATGGAACTG AAGATGAACT TCCAAATTAC TGCTTTCCAC TGGGAGGTGT |
| 20101 | GATTAATACA GAGACTCTTA CCAAGGTAAA ACCTAAAACA GGTCAGGAAA |
| 20151 | ATGGATGGGA AAAAGATGCT ACAGAATTTT CAGATAAAAA TGAAATAAGA |
| 20201 | GTTGGAAATA ATTTTGCCAT GGAAATCAAT CTAAATGCCA ACCTGTGGAG |
| 20251 | AAATTTCCTG TACTCCAACA TAGCGCTGTA TTTGCCCGAC AAGCTAAAGT |
| 20301 | ACAGTCCTTC CAACGTAAAA ATTTCTGATA ACCCAAACAC CTACGACTAC |
| 20351 | ATGAACAAGC GAGTGGTGGC TCCCGGGTTA GTGGACTGCT ACATTAACCT |
| 20401 | TGGAGCACGC TGGTCCCTTG ACTATATGGA CAACGTCAAC CCATTTAACC |
| 20451 | ACCACCGCAA TGCTGGCCTG CGCTACCGCT CAATGTTGCT GGGCAATGGT |
| 20501 | CGCTATGTGC CCTTCCACAT CCAGGTGCCT CAGAAGTTCT TTGCCATTAA |
| 20551 | AAACCTCCTT CTCCTGCCGG GCTCATACAC CTACGAGTGG AACTTCAGGA |
| 20601 | AGGATGTTAA CATGGTTCTG CAGAGCTCCC TAGGAAATGA CCTAAGGGTT |
| 20651 | GACGGAGCCA GCATTAAGTT TGATAGCATT TGCCTTTACG CCACCTTCTT |
| 20701 | CCCCATGGCC CACAACACCG CCTCCACGCT TGAGGCCATG CTTAGAAACG |
| 20751 | ACACCAACGA CCAGTCCTTT AACGACTATC TCTCCGCCGC CAACATGCTC |
| 20801 | TACCCTATAC CCGCCAACGC TACCAACGTG CCCATATCCA TCCCCTCCCG |
| 20851 | CAACTGGGCG GCTTTCCGCG GCTGGGCCTT CACGCGCCTT AAGACTAAGG |
| 20901 | AAACCCCATC ACTGGGCTCG GGCTACGACC CTTATTACAC CTACTCTGGC |
| 20951 | TCTATACCCT ACCTAGATGG AACCTTTTAC CTCAACCACA CCTTTAAGAA |
| 21001 | GGTGGCCATT ACCTTTGACT CTTCTGTCAG CTGGCCTGGC AATGACCGCC |
| 21051 | TGCTTACCCC CAACGAGTTT GAAATTAAGC GCTCAGTTGA CGGGGAGGGT |
| 21101 | TACAACGTTG CCCAGTGTAA CATGACCAAA GACTGGTTCC TGGTACAAAT |

NheI

| | |
|---|---|
| 21151 | GCTAGCTAAC TACAACATTG GCTACCAGGG CTTCTATATC CCAGAGAGCT |
| 21201 | ACAAGGACCG CATGTACTCC TTCTTTAGAA ACTTCCAGCC CATGAGCCGT |
| 21251 | CAGGTGGTGG ATGATACTAA ATACAAGGAC TACCAACAGG TGGGCATCCT |
| 21301 | ACACCAACAC AACAACTCTG GATTTGTTGG CTACCTTGCC CCCACCATGC |
| 21351 | GCGAAGGACA GGCCTACCCT GCTAACTTCC CCTATCCGCT TATAGGCAAG |
| 21401 | ACCGCAGTTG ACAGCATTAC CCAGAAAAAG TTTCTTTGCG ATCGCACCCT |
| 21451 | TTGGCGCATC CCATTCTCCA GTAACTTTAT GTCCATGGGC GCACTCACAG |
| 21501 | ACCTGGGCCA AAACCTTCTC TACGCCAACT CCGCCCACGC GCTAGACATG |

BamHI

| | |
|---|---|
| 21551 | ACTTTTGAGG TGGATCCCAT GGACGAGCCC ACCCTTCTTT ATGTTTTGTT |
| 21601 | TGAAGTCTTT GACGTGGTCC GTGTGCACCG GCCGCACCGC GGCGTCATCG |
| 21651 | AAACCGTGTA CCTGCGCACG CCCTTCTCGG CCGGCAACGC CACAACATAA |
| 21701 | AGAAGCAAGC AACATCAACA ACAGCTGCCG CCATGGGCTC CAGTGAGCAG |
| 21751 | GAACTGAAAG CCATTGTCAA AGATCTTGGT TGTGGGCCAT ATTTTTTGGG |
| 21801 | CACCTATGAC AAGCGCTTTC CAGGCTTTGT TTCTCCACAC AAGCTCGCCT |
| 21851 | GCGCCATAGT CAATACGGCC GGTCGCGAGA CTGGGGGCGT ACACTGGATG |
| 21901 | GCCTTTGCCT GGAACCCGCA CTCAAAAACA TGCTACCTCT TTGAGCCCTT |
| 21951 | TGGCTTTTCT GACCAGCGAC TCAAGCAGGT TTACCAGTTT GAGTACGAGT |
| 22001 | CACTCCTGCG CCGTAGCGCC ATTGCTTCTT CCCCCGACCG CTGTATAACG |
| 22051 | CTGGAAAAGT CCACCCAAAG CGTACAGGGG CCCAACTCGG CCGCCTGTGG |
| 22101 | ACTATTCTGC TGCATGTTTC TCCACGCCTT TGCCAACTGG CCCCAAACTC |

KpnI

| | |
|---|---|
| 22151 | CCATGGATCA CAACCCCACC ATGAACCTTA TTACCGGGGT ACCCAACTCC |
| 22201 | ATGCTCAACA GTCCCCAGGT ACAGCCCACC CTGCGTCGCA ACCAGGAACA |
| 22251 | GCTCTACAGC TTCCTGGAGC GCCACTCGCC CTACTTCCGC AGCCACAGTG |
| 22301 | CGCAGATTAG GAGCGCCACT TCTTTTTGTC ACTTGAAAAA CATGTAAAAA |
| 22351 | TAATGTACTA GAGACACTTT CAATAAAGGC AAATGCTTTT ATTTGTACAC |
| 22401 | TCTCGGGTGA TTATTTACCC CCACCCTTGC CGTCTGCGCC GTTTAAAAAT |
| 22451 | CAAAGGGGTT CTGCCGCGCA TCGCTATGCG CCACTGGCAG GGACACGTTG |
| 22501 | CGATACTGGT GTTTAGTGCT CCACTTAAAC TCAGGCACAA CCATCCGCGG |

MluI

| | |
|---|---|
| 22551 | CAGCTCGGTG AAGTTTTCAC TCCACAGGCT GCGCACCATC ACCAACGCGT |

EcoRV

| | |
|---|---|
| 22601 | TTAGCAGGTC GGGCGCCGAT ATCTTGAAGT CGCAGTTGGG GCCTCCGCCC |
| 22651 | TGCGCGCGCG AGTTGCGATA CACAGGGTTG CAGCACTGGA ACACTATCAG |
| 22701 | CGCCGGGTGG TGCACGCTGG CCAGCACGCT CTTGTCGGAG ATCAGATCCG |
| 22751 | CGTCCAGGTC CTCCGCGTTG CTCAGGGCGA ACGGAGTCAA CTTTGGTAGC |
| 22801 | TGCCTTCCCA AAAAGGGCGC GTGCCCAGGC TTTGAGTTGC ACTCGCACCG |
| 22851 | TAGTGGCATC AAAAGGTGAC CGTGCCCGGT CTGGGCGTTA GGATACAGCG |
| 22901 | CCTGCATAAA AGCCTTGATC TGCTTAAAAG CCACCTGAGC CTTTGCGCCT |
| 22951 | TCAGAGAAGA ACATGCCGCA AGACTTGCCG GAAAACTGAT TGGCCGGACA |
| 23001 | GGCCGCGTCG TGCACGCAGC ACCTTGCGTC GGTGTTGGAG ATCTGCACCA |

AgeI

| | |
|---|---|
| 23051 | CATTTCGGCC CCACCGGTTC TTCACGATCT TGGCCTTGCT AGACTGCTCC |
| 23101 | TTCAGCGCGC GCTGCCCGTT TTCGCTCGTC ACATCCATTT CAATCACGTG |
| 23151 | CTCCTTATTT ATCATAATGC TTCCGTGTAG ACACTTAAGC TCGCCTTCGA |
| 23201 | TCTCAGCGCA GCGGTGCAGC CACAACGCGC AGCCCGTGGG CTCGTGATGC |
| 23251 | TTGTAGGTCA CCTCTGCAAA CGACTGCAGG TACGCCTGCA GGAATCGCCC |
| 23301 | CATCATCGTC ACAAAGGTCT TGTTGCTGGT GAAGGTCAGC TGCAACCCGC |
| 23351 | GGTGCTCCTC GTTCAGCCAG GTCTTGCATA CGGCCGCCAG AGCTTCCACT |
| 23401 | TGGTCAGGCA GTAGTTTGAA GTTCGCCTTT AGATCGTTAT CCACGTGGTA |
| 23451 | CTTGTCCATC AGCGCGCGCG CAGCCTCCAT GCCCTTCTCC CACGCAGACA |
| 23501 | CGATCGGCAC ACTCAGCGGG TTCATCACCG TAATTTCACT TTCCGCTTCG |
| 23551 | CTGGGCTCTT CCTCTTCCTC TTGCGTCCGC ATACCACGCG CCACTGGGTC |
| 23601 | GTCTTCATTC AGCCGCCGCA CTGTGCGCTT ACCTCCTTTG CCATGCTTGA |

AgeI

| | |
|---|---|
| 23651 | TTAGCACCGG TGGGTTGCTG AAACCCACCA TTTGTAGCGC CACATCTTCT |
| 23701 | CTTTCTTCCT CGCTGTCCAC GATTACCTCT GGTGATGGCG GGCGCTCGGG |
| 23751 | CTTGGGAGAA GGGCGCTTCT TTTTCTTCTT GGGCGCAATG GCCAAATCCG |
| 23801 | CCGCCGAGGT CGATGGCCGC GGGCTGGGTG TGCGCGGCAC CAGCGCGTCT |
| 23851 | TGTGATGAGT CTTCCTCGTC CTCGGACTCG ATACGCCGCC TCATCCGCTT |
| 23901 | TTTTGGGGGC GCCCGGGGAG GCGGCGGCGA CGGGGACGGG GACGACACGT |
| 23951 | CCTCCATGGT TGGGGGACGT CGCGCCGCAC CGCGTCCGCG CTCGGGGGTG |
| 24001 | GTTTCGCGCT GCTCCTCTTC CCGACTGGCC ATTTCCTTCT CCTATAGGCA |
| 24051 | GAAAAAGATC ATGGAGTCAG TCGAGAAGAA GGACAGCCTA ACCGCCCCCT |
| 24101 | CTGAGTTCGC CACCACCGCC TCCACCGATG CCGCCAACGC GCCTACCACC |
| 24151 | TTCCCCGTCG AGGCACCCCC GCTTGAGGAG GAGGAAGTGA TTATCGAGCA |
| 24201 | GGACCCAGGT TTTGTAAGCG AAGACGACGA GGACCGCTCA GTACCAACAG |
| 24251 | AGGATAAAAA GCAAGACCAG GACAACGCAG AGGCAAACGA GGAACAAGTC |
| 24301 | GGGCGGGGGG ACGAAAGGCA TGGCGACTAC CTAGATGTGG GAGACGACGT |

MluI

| | |
|---|---|
| 24351 | GCTGTTGAAG CATCTGCAGC GCCAGTGCGC CATTATCTGC GACGCGTTGC |
| 24401 | AAGAGCGCAG CGATGTGCCC CTCGCCATAG CGGATGTCAG CCTTGCCTAC |
| 24451 | GAACGCCACC TATTCTCACC GCGCGTACCC CCCAAACGCC AAGAAAACGG |
| 24501 | CACATGCGAG CCCAACCCGC GCCTCAACTT CTACCCCGTA TTTGCCGTGC |
| 24551 | CAGAGGTGCT TGCCACCTAT CACATCTTTT TCCAAAACTG CAAGATACCC |
| 24601 | CTATCCTGCC GTGCCAACCG CAGCCGAGCG GACAAGCAGC TGGCCTTGCG |

EcoRV

| | |
|---|---|
| 24651 | GCAGGGCGCT GTCATACCTG ATATCGCCTC GCTCAACGAA GTGCCAAAAA |
| 24701 | TCTTTGAGGG TCTTGGACGC GACGAGAAGC GCGCGGCAAA CGCTCTGCAA |

XhoI

| | |
|---|---|
| 24751 | CAGGAAAACA GCGAAAATGA AAGTCACTCT GGAGTGTTGG TGGAACTCGA |
| 24801 | GGGTGACAAC GCGCGCCTAG CCGTACTAAA ACGCAGCATC GAGGTCACCC |
| 24851 | ACTTTGCCTA CCCGGCACTT AACCTACCCC CCAAGGTCAT GAGCACAGTC |
| 24901 | ATGAGTGAGC TGATCGTGCG CCGTGCGCAG CCCCTGGAGA GGGATGCAAA |

NheI

| | |
|---|---|
| 24951 | TTTGCAAGAA CAAACAGAGG AGGGCCTACC CGCAGTTGGC GACGAGCAGC |
| 25001 | TAGCGCGCTG GCTTCAAACG CGCGAGCCTG CCGACTTGGA GGAGCGACGC |
| 25051 | AAACTAATGA TGGCCGCAGT GCTCGTTACC GTGGAGCTTG AGTGCATGCA |
| 25101 | GCGGTTCTTT GCTGACCCGG AGATGCAGCG CAAGCTAGAG GAAACATTGC |
| 25151 | ACTACACCTT TCGACAGGGC TACGTACGCC AGGCCTGCAA GATCTCCAAC |
| 25201 | GTGGAGCTCT GCAACCTGGT CTCCTACCTT GGAATTTTGC ACGAAAACCG |
| 25251 | CCTTGGGCAA AACGTGCTTC ATTCCACGCT CAAGGGCGAG GCGCGCCGCG |
| 25301 | ACTACGTCCG CGACTGCGTT TACTTATTTC TATGCTACAC CTGGCAGACG |
| 25351 | GCCATGGGCG TTTGGCAGCA GTGCTTGGAG GAGTGCAACC TCAAGGAGCT |
| 25401 | GCAGAAACTG CTAAAGCAAA ACTTGAAGGA CCTATGGACG GCCTTCAACG |
| 25451 | AGCGCTCCGT GGCCGCGCAC CTGGCGGACA TCATTTTCCC CGAACGCCTG |
| 25501 | CTTAAAACCC TGCAACAGGG TCTGCCAGAC TTCACCAGTC AAAGCATGTT |
| 25551 | GCAGAACTTT AGGAACTTTA TCCTAGAGCG CTCAGGAATC TTGCCCGCCA |
| 25601 | CCTGCTGTGC ACTTCCTAGC GACTTTGTGC CCATTAAGTA CCGCGAATGC |

NheI

| | |
|---|---|
| 25651 | CCTCCGCCGC TTTGGGGCCA CTGCTACCTT CTGCAGCTAG CCAACTACCT |
| 25701 | TGCCTACCAC TCTGACATAA TGGAAGACGT GAGCGGTGAC GGTCTACTGG |
| 25751 | AGTGTCACTG TCGCTGCAAC CTATGCACCC CGCACCGCTC CCTGGTTTGC |

KpnI

| | |
|---|---|
| 25801 | AATTCGCAGC TGCTTAACGA AAGTCAAATT ATCGGTACCT TTGAGCTGCA |
| 25851 | GGGTCCCTCG CCTGACGAAA AGTCCGCGGC TCCGGGGTTG AAACTCACTC |
| 25901 | CGGGGCTGTG GACGTCGGCT TACCTTCGCA AATTTGTACC TGAGGACTAC |
| 25951 | CACGCCCACG AGATTAGGTT CTACGAAGAC CAATCCCGCC CGCCAAATGC |

MfeI

| | |
|---|---|
| 26001 | GGAGCTTACC GCCTGCGTCA TTACCCAGGG CCACATTCTT GGCCAATTGC |
| 26051 | AAGCCATCAA CAAAGCCCGC CAAGAGTTTC TGCTACGAAA GGGACGGGGG |
| 26101 | GTTTACTTGG ACCCCCAGTC CGGCGAGGAG CTCAACCCAA TCCCCCCGCC |
| 26151 | GCCGCAGCCC TATCAGCAGC AGCCGCGGGC CCTTGCTTCC CAGGATGGCA |
| 26201 | CCCAAAAAGA AGCTGCAGCT GCCGCCGCCA CCCACGGACG AGGAGGAATA |
| 26251 | CTGGGACAGT CAGGCAGAGG AGGTTTTGGA CGAGGAGGAG GAGGACATGA |

HindIII

| | |
|---|---|
| 26301 | TGGAAGACTG GGAGAGCCTA GACGAGGAAG CTTCCGAGGT CGAAGAGGTG |
| 26351 | TCAGACGAAA CACCGTCACC CTCGGTCGCA TTCCCCTCGC CGGCGCCCCA |

AgeI

| | |
|---|---|
| 26401 | GAAATCGGCA ACCGGTTCCA GCATGGCTAC AACCTCCGCT CCTCAGGCGC |
| 26451 | CGCCGGCACT GCCCGTTCGC CGACCCAACC GTAGATGGGA CACCACTGGA |
| 26501 | ACCAGGGCCG GTAAGTCCAA GCAGCCGCCG CCGTTAGCCC AAGAGCAACA |
| 26551 | ACAGCGCCAA GGCTACCGCT CATGGCGCGG GCACAAGAAC GCCATAGTTG |
| 26601 | CTTGCTTGCA AGACTGTGGG GGCAACATCT CCTTCGCCCG CCGCTTTCTT |
| 26651 | CTCTACCATC ACGGCGTGGC CTTCCCCCGT AACATCCTGC ATTACTACCG |
| 26701 | TCATCTCTAC AGCCCATACT GCACCGGCGG CAGCGGCAGC GGCAGCAACA |
| 26751 | GCAGCGGCCA CACAGAAGCA AAGGCGACCG GATAGCAAGA CTCTGACAAA |
| 26801 | GCCCAAGAAA TCCACAGCGG CGGCAGCAGC AGGAGGAGGA GCGCTGCGTC |
| 26851 | TGGCGCCCAA CGAACCCGTA TCGACCCGCG AGCTTAGAAA CAGGATTTTT |
| 26901 | CCCACTCTGT ATGCTATATT TCAACAGAGC AGGGGCCAAG AACAAGAGCT |
| 26951 | GAAAATAAAA AACAGGTCTC TGCGATCCCT CACCCGCAGC TGCCTGTATC |
| 27001 | ACAAAAGCGA AGATCAGCTT CGGCGCACGC TGGAAGACGC GGAGGCTCTC |

SpeI

| | |
|---|---|
| 27051 | TTCAGTAAAT ACTGCGCGCT GACTCTTAAG GACTAGTTTC GCGCCCTTTC |
| 27101 | TCAAATTTAA GCGCGAAAAC TACGTCATCT CCAGCGGCCA CACCCGGCGC |
| 27151 | CAGCACCTGT CGTCAGCGCC ATTATGAGCA AGGAAATTCC CACGCCCTAC |
| 27201 | ATGTGGAGTT ACCAGCCACA AATGGGACTT GCGGCTGGAG CTGCCCAAGA |

EcoRV

| | |
|---|---|
| 27251 | CTACTCAACC CGAATAAACT ACATGAGCGC GGGACCCCAC ATGATATCCC |

EcoRI

| | |
|---|---|
| 27301 | GGGTCAACGG AATCCGCGCC CACCGAAACC GAATTCTCTT GGAACAGGCG |
| 27351 | GCTATTACCA CCACACCTCG TAATAACCTT AATCCCCGTA GTTGGCCCGC |
| 27401 | TGCCCTGGTG TACCAGGAAA GTCCCGCTCC CACCACTGTG GTACTTCCCA |
| 27451 | GAGACGCCCA GGCCGAAGTT CAGATGACTA ACTCAGGGGC GCAGCTTGCG |
| 27501 | GGCGGCTTTC GTCACAGGGT GCGGTCGCCC GGGCAGGGTA TAACTCACCT |
| 27551 | GACAATCAGA GGGCGAGGTA TTCAGCTCAA CGACGAGTCG GTGAGCTCCT |
| 27601 | CGCTTGGTCT CCGTCCGGAC GGGACATTTC AGATCGGCGG CGCCGGCCGT |
| 27651 | CCTTCATTCA CGCCTCGTCA GGCAATCCTA ACTCTGCAGA CCTCGTCCTC |
| 27701 | TGAGCCGCGC TCTGGAGGCA TTGGAACTCT GCAATTTATT GAGGAGTTTG |
| 27751 | TGCCATCGGT CTACTTTAAC CCCTTCTCGG GACCTCCCGG CCACTATCCG |
| 27801 | GATCAATTTA TTCCTAACTT TGACGCGGTA AAGGACTCGG CGGACGGCTA |
| 27851 | CGACTGATTA TTAAGTGGAG AGGCAGAGCA ACTGCGCCTG AAACACCTGG |
| 27901 | TCCACTGTCG CCGCCACAAG TGCTTTGCCC GCGACTCCGG TGAGTTTTGC |
| 27951 | TACTTTGAAT TGCCCGAGGA TCATATCGAG GATCTTTGTT GCCATCTCTG |
| 28001 | TGCTGAGTAT AATAAATACA GAAATTAAAA TATACTGGGG CTCCTATCGC |
| 28051 | CATCCTGTAA ACGCCACCGT CTTCACCCGC CCAAGCAAAC CAAGGCGAAC |
| 28101 | CTTACCTGGT ACTTTTAACA TCTCTCCCTC TGTGATTTAC AACAGTTTCA |
| 28151 | ACCCAGACGG AGTGAGTCTA CGAGAGAACC TCTCCGAGCT CAGCTACTCC |
| 28201 | ATCAGAAAAA ACACCACCCT CCTTACCTGC CGGGAACGTA CCCTTATATA |

SwaI

| | |
|---|---|
| 28251 | AAAGTCAGGC TTCCTGGTGA TTAAATGAGA ATTTTAATTC GAATTTAAAT |

EcoRI KpnI BamHI XhoI

| | |
|---|---|
| 28301 | GAATTCGAGC TCGGTACCCG GGGATCCTCT AGCCCGGGCT CGAGATCTGC |
| 28351 | GATCTAAGTA AGCTCTAGAG TCGGGGCGGC CGGCCGCTTC GAGCAGACAT |
| 28401 | GATAAGATAC ATTGATGAGT TTGGACAAAC CACAACTAGA ATGCAGTGAA |
| 28451 | AAAAATGCTT TATTTGTGAA ATTTGTGATG CTATTGCTTT ATTTGTAACC |

MfeI

| | |
|---|---|
| 28501 | ATTATAAGCT GCAATAAACA AGTTAACAAC AACAATTGCA TTCATTTTAT |
| 28551 | GTTTCAGGTT CAGGGGGAGG TGTGGGAGGT TTTTTAAAGC AAGTAAAACC |

ClaI BamHI SalI

| | |
|---|---|
| 28601 | TCTACAAATG TGGTAAAATC GATAAGGATC CGTCGACCTG CAGGAAATGG |
| 28651 | AATTTCTGTC CAGTTTATTC AGCAGCACCT CCTTGCCCTC CTCCCAGCTC |
| 28701 | TGGTATTGCA GCTTCCTCCT GGCTGCAAAC TTTCTCCACA ATCTAAATGG |
| 28751 | AATGTCAGTT TCCTCCTGTT CCTGTCCATC CGCACCCACT ATCTTCATGT |
| 28801 | TGTTGCAGAT GAAGCGCGCA AGACCGTCTG AAGATACCTT CAACCCCGTG |

NdeI AgeI

| | |
|---|---|
| 28851 | TATCCATATG ACACGGAAAC CGGTCCTCCA ACTGTGCCTT TTCTTACTCC |
| 28901 | TCCCTTTGTA TCCCCCAATG GGTTTCAAGA GAGTCCCCCT GGGGTACTCT |
| 28951 | CTTTGCGCCT ATCCGAACCT CTAGTTACCT CCAATGGCAT GCTTGCGCTC |
| 29001 | AAAATGGGCA ACGGCCTCTC TCTGGACGAG GCCGGCAACC TTACCTCCCA |
| 29051 | AAATGTAACC ACTGTGAGCC CACCTCTCAA AAAAACCAAG TCAAACATAA |
| 29101 | ACCTGGAAAT ATCTGCACCC CTCACAGTTA CCTCAGAAGC CCTAACTGTG |
| 29151 | GCTGCCGCCG CACCTCTAAT GGTCGCGGGC AACACACTCA CCATGCAATC |
| 29201 | ACAGGCCCCG CTAACCGTGC ACGACTCCAA ACTTAGCATT GCCACCCAAG |

NheI

| | |
|---|---|
| 29251 | GACCCCTCAC AGTGTCAGAA GGAAAGCTAG CCCTGCAAAC ATCAGGCCCC |
| 29301 | CTCACCACCA CCGATAGCAG TACCCTTACT ATCACTGCCT CACCCCCTCT |
| 29351 | AACTACTGCC ACTGGTAGCT TGGGCATTGA CTTGAAAGAG CCCATTTATA |
| 29401 | CACAAAATGG AAAACTAGGA CTAAAGTACG GGGCTCCTTT GCATGTAACA |
| 29451 | GACGACCTAA ACACTTTGAC CGTAGCAACT GGTCCAGGTG TGACTATTAA |
| 29501 | TAATACTTCC TTGCAAACTA AAGTTACTGG AGCCTTGGGT TTTGATTCAC |
| 29551 | AAGGCAATAT GCAACTTAAT GTAGCAGGAG GACTAAGGAT TGATTCTCAA |
| 29601 | AACAGACGCC TTATACTTGA TGTTAGTTAT CCGTTTGATG CTCAAAACCA |
| 29651 | ACTAAATCTA AGACTAGGAC AGGGCCCTCT TTTTATAAAC TCAGCCCACA |
| 29701 | ACTTGGATAT TAACTACAAC AAAGGCCTTT ACTTGTTTAC AGCTTCAAAC |

HindIII

| | |
|---|---|
| 29751 | AATTCCAAAA AGCTTGAGGT TAACCTAAGC ACTGCCAAGG GGTTGATGTT |
| 29801 | TGACGCTACA GCCATAGCCA TTAATGCAGG AGATGGGCTT GAATTTGGTT |
| 29851 | CACCTAATGC ACCAAACACA AATCCCCTCA AAACAAAAAT TGGCCATGGC |
| 29901 | CTAGAATTTG ATTCAAACAA GGCTATGGTT CCTAAACTAG GAACTGGCCT |
| 29951 | TAGTTTTGAC AGCACAGGTG CCATTACAGT AGGAAACAAA AATAATGATA |
| 30001 | AGCTAACTTT GTGGACCACA CCAGCTCCAT CTCCTAACTG TAGACTAAAT |
| 30051 | GCAGAGAAAG ATGCTAAACT CACTTTGGTC TTAACAAAAT GTGGCAGTCA |
| 30101 | AATACTTGCT ACAGTTTCAG TTTTGGCTGT TAAAGGCAGT TTGGCTCCAA |
| 30151 | TATCTGGAAC AGTTCAAAGT GCTCATCTTA TTATAAGATT TGACGAAAAT |
| 30201 | GGAGTGCTAC TAAACAATTC CTTCCTGGAC CCAGAATATT GGAACTTTAG |
| 30251 | AAATGGAGAT CTTACTGAAG GCACAGCCTA TACAAACGCT GTTGGATTTA |
| 30301 | TGCCTAACCT ATCAGCTTAT CCAAAATCTC ACGGTAAAAC TGCCAAAAGT |
| 30351 | AACATTGTCA GTCAAGTTTA CTTAAACGGA GACAAAACTA AACCTGTAAC |
| 30401 | ACTAACCATT ACACTAAACG GTACACAGGA AACAGGAGAC ACAACTCCAA |
| 30451 | GTGCATACTC TATGTCATTT TCATGGGACT GGTCTGGCCA CAACTACATT |
| 30501 | AATGAAATAT TTGCCACATC CTCTTACACT TTTTCATACA TTGCCCAAGA |

MfeI

| | |
|---|---|
| 30551 | ATAAAGAATC GTTTGTGTTA TGTTTCAACG TGTTTATTTT TCAATTGCAG |
| 30601 | AAAATTTCAA GTCATTTTTC ATTCAGTAGT ATAGCCCCAC CACCACATAG |
| 30651 | CTTATACAGA TCACCGTACC TTAATCAAAC TCACAGAACC CTAGTATTCA |
| 30701 | ACCTGCCACC TCCCTCCCAA CACACAGAGT ACACAGTCCT TTCTCCCCGG |
| 30751 | CTGGCCTTAA AAAGCATCAT ATCATGGGTA ACAGACATAT TCTTAGGTGT |
| 30801 | TATATTCCAC ACGGTTTCCT GTCGAGCCAA ACGCTCATCA GTGATATTAA |
| 30851 | TAAACTCCCC GGGCAGCTCA CTTAAGTTCA TGTCGCTGTC CAGCTGCTGA |
| 30901 | GCCACAGGCT GCTGTCCAAC TTGCGGTTGC TTAACGGGCG GCGAAGGAGA |
| 30951 | AGTCCACGCC TACATGGGGG TAGAGTCATA ATCGTGCATC AGGATAGGGC |
| 31001 | GGTGGTGCTG CAGCAGCGCG CGAATAAACT GCTGCCGCCG CCGCTCCGTC |
| 31051 | CTGCAGGAAT ACAACATGGC AGTGGTCTCC TCAGCGATGA TTCGCACCGC |
| 31101 | CCGCAGCATA AGGCGCCTTG TCCTCCGGGC ACAGCAGCGC ACCCTGATCT |
| 31151 | CACTTAAATC AGCACAGTAA CTGCAGCACA GCACCACAAT ATTGTTCAAA |
| 31201 | ATCCCACAGT GCAAGGCGCT GTATCCAAAG CTCATGGCGG GGACCACAGA |
| 31251 | ACCCACGTGG CCATCATACC ACAAGCGCAG GTAGATTAAG TGGCGACCCC |
| 31301 | TCATAAACAC GCTGGACATA AACATTACCT CTTTTGGCAT GTTGTAATTC |

KpnI

| | |
|---|---|
| 31351 | ACCACCTCCC GGTACCATAT AAACCTCTGA TTAAACATGG CGCCATCCAC |
| 31401 | CACCATCCTA AACCAGCTGG CCAAAACCTG CCCGCCGGCT ATACACTGCA |
| 31451 | GGGAACCGGG ACTGGAACAA TGACAGTGGA GAGCCCAGGA CTCGTAACCA |

EcoRV

| | |
|---|---|
| 31501 | TGGATCATCA TGCTCGTCAT GATATCAATG TTGGCACAAC ACAGGCACAC |
| 31551 | GTGCATACAC TTCCTCAGGA TTACAAGCTC CTCCCGCGTT AGAACCATAT |
| 31601 | CCCAGGGAAC AACCCATTCC TGAATCAGCG TAAATCCCAC ACTGCAGGGA |
| 31651 | AGACCTCGCA CGTAACTCAC GTTGTGCATT GTCAAAGTGT TACATTCGGG |
| 31701 | CAGCAGCGGA TGATCCTCCA GTATGGTAGC GCGGGTTTCT GTCTCAAAAG |
| 31751 | GAGGTAGACG ATCCCTACTG TACGGAGTGC GCCGAGACAA CCGAGATCGT |
| 31801 | GTTGGTCGTA GTGTCATGCC AAATGGAACG CCGGACGTAG TCATATTTCC |
| 31851 | TGAAGCAAAA CCAGGTGCGG GCGTGACAAA CAGATCTGCG TCTCCGGTCT |
| 31901 | CGCCGCTTAG ATCGCTCTGT GTAGTAGTTG TAGTATATCC ACTCTCTCAA |
| 31951 | AGCATCCAGG CGCCCCCTGG CTTCGGGTTC TATGTAAACT CCTTCATGCG |
| 32001 | CCGCTGCCCT GATAACATCC ACCACCGCAG AATAAGCCAC ACCCAGCCAA |
| 32051 | CCTACACATT CGTTCTGCGA GTCACACACG GGAGGAGCGG GAAGAGCTGG |
| 32101 | AAGAACCATG TTTTTTTTTT TATTCCAAAA GATTATCCAA AACCTCAAAA |
| 32151 | TGAAGATCTA TTAAGTGAAC GCGCTCCCCT CCGGTGGCGT GGTCAAACTC |
| 32201 | TACAGCCAAA GAACAGATAA TGGCATTTGT AAGATGTTGC ACAATGGCTT |
| 32251 | CCAAAAGGCA AACGGCCCTC ACGTCCAAGT GGACGTAAAG GCTAAACCCT |
| 32301 | TCAGGGTGAA TCTCCTCTAT AAACATTCCA GCACCTTCAA CCATGCCCAA |
| 32351 | ATAATTCTCA TCTCGCCACC TTCTCAATAT ATCTCTAAGC AAATCCCGAA |
| 32401 | TATTAAGTCC GGCCATTGTA AAAATCTGCT CCAGAGCGCC CTCCACCTTC |
| 32451 | AGCCTCAAGC AGCGAATCAT GATTGCAAAA ATTCAGGTTC CTCACAGACC |
| 32501 | TGTATAAGAT TCAAAAGCGG AACATTAACA AAAATACCGC GATCCCGTAG |
| 32551 | GTCCCTTCGC AGGGCCAGCT GAACATAATC GTGCAGGTCT GCACGGACCA |
| 32601 | GCGCGGCCAC TTCCCCGCCA GGAACCATGA CAAAAGAACC CACACTGATT |

HindIII

| | |
|---|---|
| 32651 | ATGACACGCA TACTCGGAGC TATGCTAACC AGCGTAGCCC CGATGTAAGC |
| 32701 | TTGTTGCATG GGCGGCGATA TAAAATGCAA GGTGCTGCTC AAAAAATCAG |
| 32751 | GCAAAGCCTC GCGCAAAAAA GAAAGCACAT CGTAGTCATG CTCATGCAGA |
| 32801 | TAAAGGCAGG TAAGCTCCGG AACCACCACA GAAAAAGACA CCATTTTTCT |
| 32851 | CTCAAACATG TCTGCGGGTT TCTGCATAAA CACAAAATAA AATAACAAAA |
| 32901 | AAACATTTAA ACATTAGAAG CCTGTCTTAC AACAGGAAAA ACAACCCTTA |
| 32951 | TAAGCATAAG ACGGACTACG GCCATGCCGG CGTGACCGTA AAAAAACTGG |
| 33001 | TCACCGTGAT TAAAAAGCAC CACCGACAGC TCCTCGGTCA TGTCCGGAGT |
| 33051 | CATAATGTAA GACTCGGTAA ACACATCAGG TTGATTCACA TCGGTCAGTG |
| 33101 | CTAAAAAGCG ACCGAAATAG CCCGGGGGAA TACATACCCG CAGGCGTAGA |
| 33151 | GACAACATTA CAGCCCCCAT AGGAGGTATA ACAAAATTAA TAGGAGAGAA |
| 33201 | AAACACATAA ACACCTGAAA AACCCTCCTG CCTAGGCAAA ATAGCACCCT |
| 33251 | CCCGCTCCAG AACAACATAC AGCGCTTCCA CAGCGGCAGC CATAACAGTC |
| 33301 | AGCCTTACCA GTAAAAAAGA AAACCTATTA AAAAAACACC ACTCGACACG |
| 33351 | GCACCAGCTC AATCAGTCAC AGTGTAAAAA AGGGCCAAGT GCAGAGCGAG |
| 33401 | TATATATAGG ACTAAAAAAT GACGTAACGG TTAAAGTCCA CAAAAAACAC |
| 33451 | CCAGAAAACC GCACGCGAAC CTACGCCCAG AAACGAAAGC CAAAAAACCC |
| 33501 | ACAACTTCCT CAAATCGTCA CTTCCGTTTT CCCACGTTAC GTCACTTCCC |
| 33551 | ATTTTAAGAA AACTACAATT CCCAACACAT ACAAGTTACT CCGCCCTAAA |
| 33601 | ACCTACGTCA CCCGCCCCGT TCCCACGCCC CGCGCCACGT CACAAACTCC |
| 33651 | ACCCCCTCAT TATCATATTG GCTTCAATCC AAAATAAGGT ATATTATTGA |

PacI EcoRI

| | |
|---|---|
| 33701 | TGATGTTAAT TAAGAATTCG GATCTGCGAC GCGAGGCTGG ATGGCCTTCC |
| 33751 | CCATTATGAT TCTTCTCGCT TCCGGCGGCA TCGGGATGCC CGCGTTGCAG |
| 33801 | GCCATGCTGT CCAGGCAGGT AGATGACGAC CATCAGGGAC AGCTTCACGG |
| 33851 | CCAGCAAAAG GCCAGGAACC GTAAAAAGGC CGCGTTGCTG GCGTTTTTCC |
| 33901 | ATAGGCTCCG CCCCCCTGAC GAGCATCACA AAAATCGACG CTCAAGTCAG |
| 33951 | AGGTGGCGAA ACCCGACAGG ACTATAAAGA TACCAGGCGT TTCCCCCTGG |
| 34001 | AAGCTCCCTC GTGCGCTCTC CTGTTCCGAC CCTGCCGCTT ACCGGATACC |
| 34051 | TGTCCGCCTT TCTCCCTTCG GGAAGCGTGG CGCTTTCTCA ATGCTCACGC |
| 34101 | TGTAGGTATC TCAGTTCGGT GTAGGTCGTT CGCTCCAAGC TGGGCTGTGT |
| 34151 | GCACGAACCC CCCGTTCAGC CCGACCGCTG CGCCTTATCC GGTAACTATC |
| 34201 | GTCTTGAGTC CAACCCGGTA AGACACGACT TATCGCCACT GGCAGCAGCC |
| 34251 | ACTGGTAACA GGATTAGCAG AGCGAGGTAT GTAGGCGGTG CTACAGAGTT |
| 34301 | CTTGAAGTGG TGGCCTAACT ACGGCTACAC TAGAAGGACA GTATTTGGTA |
| 34351 | TCTGCGCTCT GCTGAAGCCA GTTACCTTCG GAAAAAGAGT TGGTAGCTCT |
| 34401 | TGATCCGGCA AACAAACCAC CGCTGGTAGC GGTGGTTTTT TTGTTTGCAA |
| 34451 | GCAGCAGATT ACGCGCAGAA AAAAAGGATC TCAAGAAGAT CCTTTGATCT |
| 34501 | TTTCTACGGG GTCTGACGCT CAGTGGAACG AAAACTCACG TTAAGGGATT |
| 34551 | TTGGTCATGA GATTATCAAA AAGGATCTTC ACCTAGATCC TTTTAAATCA |
| 34601 | ATCTAAAGTA TATATGAGTA AACTTGGTCT GACAGTTACC AATGCTTAAT |
| 34651 | CAGTGAGGCA CCTATCTCAG CGATCTGTCT ATTTCGTTCA TCCATAGTTG |
| 34701 | CCTGACTCCC CGTCGTGTAG ATAACTACGA TACGGGAGGG CTTACCATCT |
| 34751 | GGCCCCAGTG CTGCAATGAT ACCGCGAGAC CCACGCTCAC CGGCTCCAGA |
| 34801 | TTTATCAGCA ATAAACCAGC CAGCCGGAAG GGCCGAGCGC AGAAGTGGTC |
| 34851 | CTGCAACTTT ATCCGCCTCC ATCCAGTCTA TTAATTGTTG CCGGGAAGCT |
| 34901 | AGAGTAAGTA GTTCGCCAGT TAATAGTTTG CGCAACGTTG TTGCCATTGN |
| 34951 | TGCAGGCATC GTGGTGTCAC GCTCGTCGTT TGGTATGGCT TCATTCAGCT |
| 45001 | CCGGTTCCCA ACGATCAAGG CGAGTTACAT GATCCCCCAT GTGGTGCAAA |
| 35051 | AAAGCGGTTA GCTCCTTCGG TCCTCCGATC GTTGTCAGAA GTAAGTTGGC |
| 35101 | CGCAGTGTTA TCACTCATGG TTATGGCAGC ACTGCATAAT TCTCTTACTG |
| 35151 | TCATGCCATC CGTAAGATGC TTTTCTGTGA CTGGTGAGTA CTCAACCAAG |
| 35201 | TCATTCTGAG AATAGTGTAT GCGGCGACCG AGTTGCTCTT GCCCGGCGTC |
| 35251 | AACACGGGAT AATACCGCGC CACATAGCAG AACTTTAAAA GTGCTCATCA |
| 35301 | TTGGAAAACG TTCTTCGGGG CGAAAACTCT CAAGGATCTT ACCGCTGTTG |
| 35351 | AGATCCAGTT CGATGTAACC CACTCGTGCA CCCAACTGAT CTTCAGCATC |
| 35401 | TTTTACTTTC ACCAGCGTTT CTGGGTGAGC AAAAACAGGA AGGCAAAATG |
| 35451 | CCGCAAAAAA GGGAATAAGG GCGACACGGA AATGTTGAAT ACTCATACTC |
| 35501 | TTCCTTTTTC AATATTATTG AAGCATTTAT CAGGGTTATT GTCTCATGAG |
| 35551 | CGGATACATA TTTGAATGTA TTTAGAAAAA TAAACAAATA GGGGTTCCGC |
| 35601 | GCACATTTCC CCGAAAAGTG CCACCTGACG TCTAAGAAAC CATTATTATC |
| 35651 | ATGACATTAA CCTATAAAAA TAGGCGTATC ACGAGGCCCT TTCGTCTTCA |

BamHI EcoRI PacI

| | |
|---|---|
| 35701 | AGGATCCGAA TTCTTAATTA A |

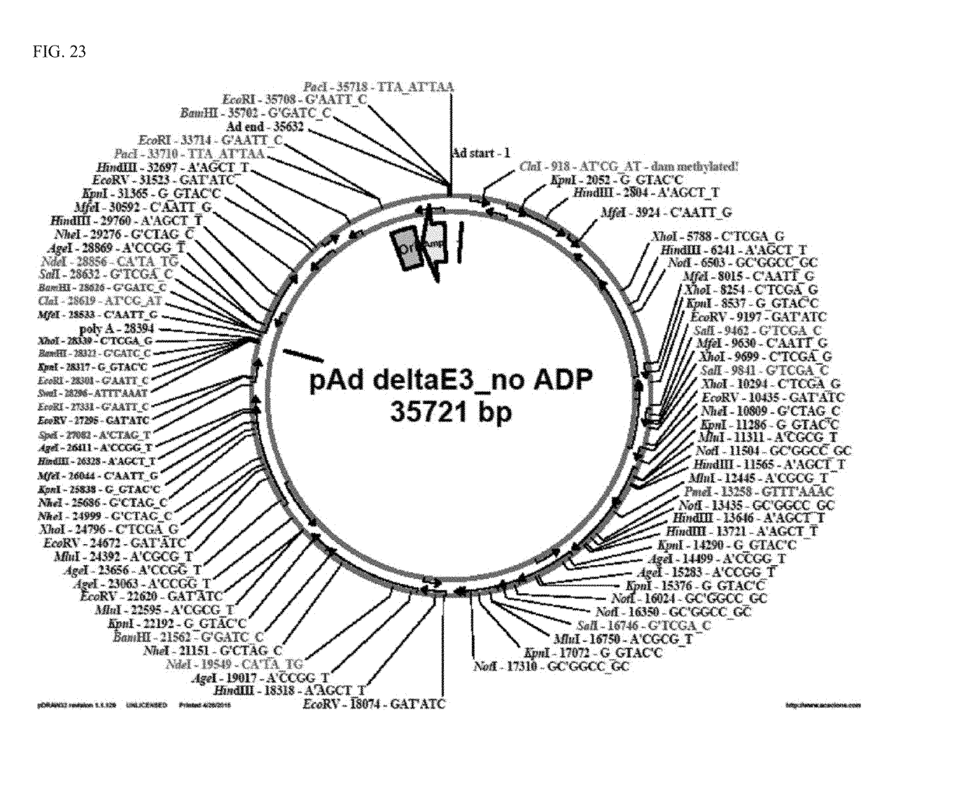

SEQ ID NO: 2
Ad5/Ad3 Cox2-ΔE3/ΔADP-NIS vector (FIG. 23). This vector is based
on the ΔE3/ΔADP structure with the following interchangeable
parts:
Tumor Specific Promoter (TSP): Cox2
Adenovirus fiber: Ad5/Ad3
Therapeutic/Imaging Transgene: NIS
Adenovirus E3 region structure: deletion of ADP

| | |
|---|---|
| 1 | TTAACATCAT CAATAATATA CCTTATTTTG GATTGAAGCC AATATGATAA |
| 51 | TGAGGGGGTG GAGTTTGTGA CGTGGCGCGG GGCGTGGGAA CGGGGCGGGT |
| 101 | GACGTAGTAG TGTGGCGGAA GTGTGATGTT GCAAGTGTGG CGGAACACAT |
| 151 | GTAAGCGACG GATGTGGCAA AAGTGACGTT TTTGGTGTGC GCCGGTGTAC |
| 201 | ACAGGAAGTG ACAATTTTCG CGCGGTTTTA GGCGGATGTT GTAGTAAATT |
| 251 | TGGGCGTAAC CGAGTAAGAT TTGGCCATTT TCGCGGGAAA ACTGAATAAG |
| 301 | AGGAAGTGAA ATCTGAATAA TTTTGTGTTA CTCATAGCGC GTAATACTGG |
| 351 | TACGGGGTCG ACCCCGTACC TGGTGTAGTT TTATTTCAGG TTTTATACTG |
| 401 | TCATTTTCCT GTAATGCTAA GGACTTAGGA CATAACTGAA TTTTCTATTT |
| 451 | TCCACTTCTT TTCTGGTGTG TGTGTATATA TATATGTATA TATACACACA |
| 501 | CACATATACA TATATATATT TTTTAGTATC TCACCCTCAC ATGCTCCTCC |
| 551 | CTGAGCACTA CCCATGATAG ATGTTAAACA AAAGCAAAGA TGAAATTCCA |
| 601 | ACTGTCAAAA TCTCCCTTCC ATCTAATTAA TTCCTCATCC AACTATGTTC |
| 651 | CAAAACGAGA ATAGAAAATT AGCCCCAATA AGCCCAGGCA ACTGAAAAGT |
| 701 | AAATGCTATG TTGTACTTTG ATCCATGGTC ACAACTCATA ATCTTGGAAA |
| 751 | AGTGGACAGA AAAGACAAAA GAGTGAACTT TAAAACTCGA ATTTATTTTA |
| 801 | CCAGTATCTC CTATGAAGGG CTAGTAACCA AAATAATCCA CGCATCAGGG |
| 851 | AGAGAAATGC CTTAAGGCAT ACGTTTTGGA CATTTAGCGT CCCTGCAAAT |
| 901 | TCTGGCCATC GCCGCTTCCT TTGTCCATCA GAAGGCAGGA AACTTTATAT |
| 951 | TGGTGACCCG TGGAGCTCAC ATTAACTATT TACAGGGTAA CTGCTTAGGA |
| 1001 | CCAGTATTAT GAGGAGAATT TACCTTTCCC GCCTCTCTTT CCAAGAAACA |
| 1051 | AGGAGGGGGT GAAGGTACGG AGAACAGTAT TTCTTCTGTT GAAAGCAACT |
| 1101 | TAGCTACAAA GATAAATTAC AGCTATGTAC ACTGAAGGTA GCTATTTCAT |
| 1151 | TCCACAAAAT AAGAGTTTTT TAAAAAGCTA TGTATGTATG TCCTGCATAT |
| 1201 | AGAGCAGATA TACAGCCTAT TAAGCGTCGT CACTAAAACA TAAAACATGT |
| 1251 | CAGCCTTTCT TAACCTTACT CGCCCCAGTC TGTCCCGACG TGACTTCCTC |
| 1301 | GACCCTCTAA AGACGTACAG ACCAGACACG GCGGCGGTGG CGGGAGAGGG |
| 1351 | GATTCCCTGC GCCCCCGGAC CTCAGGGCCG CTCAGATTCC TGGAGAGGAA |
| 1401 | GCCAAGTGTC CTTCTGCCCT CCCCCGGTAT CCCATCCAAG GCGATCAGTC |
| 1451 | CAGAACTGGC TCTCGGAAGC GCTCGGGCAA AGACTGCGAA GAAGAAAAGA |
| 1501 | CATCTGGCGG AAACCTGTGC GCCTGGGGCG GTGGAACTCG GGGAGGAGAG |
| 1551 | GGAGGGATCA GACAGGAGAG TGGGGACTAC CCCCTCTGCT CCCAAATTGG |
| 1601 | GGCAGCTTCC TGGGTTTCCG ATTTTCTCAT TTCCGTGGGT AAAAAACCCT |
| 1651 | GCCCCCACCG GGCTTACGCA ATTTTTTTAA GGGGAGAGGA GGGAAAAATT |
| 1701 | TGTGGGGGGT ACGAAAAGGC GGAAAGAAAC AGTCATTTCG TCACATGGGC |
| 1751 | TTGGTTTTCA GTCTTATAAA AAGGAAGGTT CTCTCGGTTA GCGACCAATT |
| 1801 | GTCATACGAC TTGCAGTGAG CGTCAGGAGC ACGTCCAGGA ACTCCTCAGC |
| 1851 | AGGGCTGCAG GAATTCGATA TCAAGCTTGA AAATGAGACA TATTATCTGC |
| 1901 | CACGGAGGTG TTATTACCGA AGAAATGGCC GCCAGTCTTT TGGACCAGCT |
| 1951 | GATCGAAGAG GTACTGGCTG ATAATCTTCC ACCTCCTAGC CATTTTGAAC |
| 2001 | CACCTACCCT TCACGAACTG TATGATTTAG ACGTGACGGC CCCCGAAGAT |
| 2051 | CCCAACGAGG AGGCGGTTTC GCAGATTTTT CCCGACTCTG TAATGTTGGC |
| 2101 | GGTGCAGGAA GGGATTGACT TACTCACTTT TCCGCCGGCG CCCGGTTCTC |
| 2151 | CGGAGCCGCC TCACCTTTCC CGGCAGCCCG AGCAGCCGGA GCAGAGAGCC |
| 2201 | TTGGGTCCGG TTTCTATGCC AAACCTTGTA CCGGAGGTGA TCGATCTTAC |
| 2251 | CTGCCACGAG GCTGGCTTTC CACCCAGTGA CGACGAGGAT GAAGAGGGTG |
| 2301 | AGGAGTTTGT GTTAGATTAT GTGGAGCACC CCGGGCACGG TTGCAGGTCT |
| 2351 | TGTCATTATC ACCGGAGGAA TACGGGGGAC CCAGATATTA TGTGTTCGCT |
| 2401 | TTGCTATATG AGGACCTGTG GCATGTTTGT CTACAGTAAG TGAAAATTAT |
| 2451 | GGGCAGTGGG TGATAGAGTG GTGGGTTTGG TGTGGTAATT TTTTTTTTAA |
| 2501 | TTTTTACAGT TTTGTGGTTT AAAGAATTTT GTATTGTGAT TTTTTTAAAA |
| 2551 | GGTCCTGTGT CTGAACCTGA GCCTGAGCCC GAGCCAGAAC CGGAGCCTGC |
| 2601 | AAGACCTACC CGCCGTCCTA AAATGGCGCC TGCTATCCTG AGACGCCCGA |
| 2651 | CATCACCTGT GTCTAGAGAA TGCAATAGTA GTACGGATAG CTGTGACTCC |
| 2701 | GGTCCTTCTA ACACACCTCC TGAGATACAC CCGGTGGTCC CGCTGTGCCC |
| 2751 | CATTAAACCA GTTGCCGTGA GAGTTGGTGG GCGTCGCCAG GCTGTGGAAT |
| 2801 | GTATCGAGGA CTTGCTTAAC GAGCCTGGGC AACCTTTGGA CTTGAGCTGT |
| 2851 | AAACGCCCCA GGCCATAAGG TGTAAACCTG TGATTGCGTG TGTGGTTAAC |
| 2901 | GCCTTTGTTT GCTGAATGAG TTGATGTAAG TTTAATAAAG GGTGAGATAA |
| 2951 | TGTTTAACTT GCATGGCGTG TTAAATGGGG CGGGGCTTAA AGGGTATATA |
| 3001 | ATGCGCCGTG GGCTAATCTT GGTTACATCT GACCTCATGG AGGCTTGGGA |
| 3051 | GTGTTTGGAA GATTTTTCTG CTGTGCGTAA CTTGCTGGAA CAGAGCTCTA |
| 3101 | ACAGTACCTC TTGGTTTTGG AGGTTTCTGT GGGGCTCATC CCAGGCAAAG |
| 3151 | TTAGTCTGCA GAATTAAGGA GGATTACAAG TGGGAATTTG AAGAGCTTTT |
| 3201 | GAAATCCTGT GGTGAGCTGT TTGATTCTTT GAATCTGGGT CACCAGGCGC |
| 3251 | TTTTCCAAGA GAAGGTCATC AAGACTTTGG ATTTTTCCAC ACCGGGGCGC |
| 3301 | GCTGCGGCTG CTGTTGCTTT TTTGAGTTTT ATAAAGGATA AATGGAGCGA |
| 3351 | AGAAACCCAT CTGAGCGGGG GGTACCTGCT GGATTTTCTG GCCATGCATC |
| 3401 | TGTGGAGAGC GGTTGTGAGA CACAAGAATC GCCTGCTACT GTTGTCTTCC |
| 3451 | GTCCGCCCGG CGATAATACC GACGGAGGAG CAGCAGCAGC AGCAGGAGGA |
| 3501 | AGCCAGGCGG CGGCGGCAGG AGCAGAGCCC ATGGAACCCG AGAGCCGGCC |
| 3551 | TGGACCCTCG GGAATGAATG TTGTACAGGT GGCTGAACTG TATCCAGAAC |
| 3601 | TGAGACGCAT TTTGACAATT ACAGAGGATG GGCAGGGGCT AAAGGGGGTA |
| 3651 | AAGAGGGAGC GGGGGGCTTG TGAGGCTACA GAGGAGGCTA GGAATCTAGC |
| 3701 | TTTTAGCTTA ATGACCAGAC ACCGTCCTGA GTGTATTACT TTTCAACAGA |
| 3751 | TCAAGGATAA TTGCGCTAAT GAGCTTGATC TGCTGGCGCA GAAGTATTCC |
| 3801 | ATAGAGCAGC TGACCACTTA CTGGCTGCAG CCAGGGGATG ATTTTGAGGA |
| 3851 | GGCTATTAGG GTATATGCAA AGGTGGCACT TAGGCCAGAT TGCAAGTACA |
| 3901 | AGATCAGCAA ACTTGTAAAT ATCAGGAATT GTTGCTACAT TTCTGGGAAC |
| 3951 | GGGGCCGAGG TGGAGATAGA TACGGAGGAT AGGGTGGCCT TTAGATGTAG |
| 4001 | CATGATAAAT ATGTGGCCGG GGGTGCTTGG CATGGACGGG GTGGTTATTA |
| 4051 | TGAATGTAAG GTTTACTGGC CCCAATTTTA GCGGTACGGT TTTCCTGGCC |
| 4101 | AATACCAACC TTATCCTACA CGGTGTAAGC TTCTATGGGT TTAACAATAC |
| 4151 | CTGTGTGGAA GCCTGGACCG ATGTAAGGGT TCGGGGCTGT GCCTTTTACT |
| 4201 | GCTGCTGGAA GGGGGTGGTG TGTCGCCCCA AAAGCAGGGC TTCAATTAAG |
| 4251 | AAATGCCTCT TTGAAAGGTG TACCTTGGGT ATCCTGTCTG AGGGTAACTC |
| 4301 | CAGGGTGCGC CACAATGTGG CCTCCGACTG TGGTTGCTTC ATGCTAGTGA |
| 4351 | AAAGCGTGGC TGTGATTAAG CATAACATGG TATGTGGCAA CTGCGAGGAC |
| 4401 | AGGGCCTCTC AGATGCTGAC CTGCTCGGAC GGCAACTGTC ACCTGCTGAA |
| 4451 | GACCATTCAC GTAGCCAGCC ACTCTCGCAA GGCCTGGCCA GTGTTTGAGC |
| 4501 | ATAACATACT GACCCGCTGT TCCTTGCATT TGGGTAACAG GAGGGGGGTG |
| 4551 | TTCCTACCTT ACCAATGCAA TTTGAGTCAC ACTAAGATAT TGCTTGAGCC |
| 4601 | CGAGAGCATG TCCAAGGTGA ACCTGAACGG GGTGTTTGAC ATGACCATGA |
| 4651 | AGATCTGGAA GGTGCTGAGG TACGATGAGA CCCGCACCAG GTGCAGACCC |
| 4701 | TGCGAGTGTG GCGGTAAACA TATTAGGAAC CAGCCTGTGA TGCTGGATGT |
| 4751 | GACCGAGGAG CTGAGGCCCG ATCACTTGGT GCTGGCCTGC ACCCGCGCTG |
| 4801 | AGTTTGGCTC TAGCGATGAA GATACAGATT GAGGTTCTAG AGTCGGGGCG |
| 4851 | GCCGGCCGCT TCGAGCAGAC ATGATAAGAT ACATTGATGA GTTTGGACAA |
| 4901 | ACCACAACTA GAATGCAGTG AAAAAAATGC TTTATTTGTG AAATTTGTGA |
| 4951 | TGCTATTGCT TTATTTGTAA CCATTATAAG CTGCAATAAA CAAGTTAACA |
| 5001 | ACAACAATTG CATTCATTTT ATGTTTCAGG TTCAGGGGGA GGTGTGGGAG |
| 5051 | GTTTTTTAAA GCAAGTAAAA CCTCTACAAA TGTGGTAAAA TCGATAAGGA |
| 5101 | TCCGTCGACT CGAAGATCTT GTACTGAAAT GTGTGGGCGT GGCTTAAGGG |
| 5151 | TGGGAAAGAA TATATAAGGT GGGGGTCTTA TGTAGTTTTG TATCTGTTTT |
| 5201 | GCAGCAGCCG CCGCCGCCAT GAGCACCAAC TCGTTTGATG GAAGCATTGT |
| 5251 | GAGCTCATAT TTGACAACGC GCATGCCCCC ATGGGCCGGG GTGCGTCAGA |
| 5301 | ATGTGATGGG CTCCAGCATT GATGGTCGCC CCGTCCTGCC CGCAAACTCT |
| 5351 | ACTACCTTGA CCTACGAGAC CGTGTCTGGA ACGCCGTTGG AGACTGCAGC |
| 5401 | CTCCGCCGCC GCTTCAGCCG CTGCAGCCAC CGCCCGCGGG ATTGTGACTG |
| 5451 | ACTTTGCTTT CCTGAGCCCG CTTGCAAGCA GTGCAGCTTC CCGTTCATCC |
| 5501 | GCCCGCGATG ACAAGTTGAC GGCTCTTTTG GCACAATTGG ATTCTTTGAC |
| 5551 | CCGGGAACTT AATGTCGTTT CTCAGCAGCT GTTGGATCTG CGCCAGCAGG |
| 5601 | TTTCTGCCCT GAAGGCTTCC TCCCCTCCCA ATGCGGTTTA AAACATAAAT |
| 5651 | AAAAAACCAG ACTCTGTTTG GATTTGGATC AAGCAAGTGT CTTGCTGTCT |
| 5701 | TTATTTAGGG GTTTTGCGCG CGCGGTAGGC CCGGGACCAG CGGTCTCGGT |
| 5751 | CGTTGAGGGT CCTGTGTATT TTTTCCAGGA CGTGGTAAAG GTGACTCTGG |
| 5801 | ATGTTCAGAT ACATGGGCAT AAGCCCGTCT CTGGGGTGGA GGTAGCACCA |
| 5851 | CTGCAGAGCT TCATGCTGCG GGGTGGTGTT GTAGATGATC CAGTCGTAGC |
| 5901 | AGGAGCGCTG GGCGTGGTGC CTAAAAATGT CTTTCAGTAG CAAGCTGATT |
| 5951 | GCCAGGGGCA GGCCCTTGGT GTAAGTGTTT ACAAAGCGGT TAAGCTGGGA |
| 6001 | TGGGTGCATA CGTGGGGATA TGAGATGCAT CTTGGACTGT ATTTTTAGGT |
| 6051 | TGGCTATGTT CCCAGCCATA TCCCTCCGGG GATTCATGTT GTGCAGAACC |
| 6101 | ACCAGCACAG TGTATCCGGT GCACTTGGGA AATTTGTCAT GTAGCTTAGA |
| 6151 | AGGAAATGCG TGGAAGAACT TGGAGACGCC CTTGTGACCT CCAAGATTTT |
| 6201 | CCATGCATTC GTCCATAATG ATGGCAATGG GCCCACGGGC GGCGGCCTGG |
| 6251 | GCGAAGATAT TTCTGGGATC ACTAACGTCA TAGTTGTGTT CCAGGATGAG |
| 6301 | ATCGTCATAG GCCATTTTTA CAAAGCGCGG GCGGAGGGTG CCAGACTGCG |
| 6351 | GTATAATGGT TCCATCCGGC CCAGGGGCGT AGTTACCCTC ACAGATTTGC |
| 6401 | ATTTCCCACG CTTTGAGTTC AGATGGGGGG ATCATGTCTA CCTGCGGGGC |
| 6451 | GATGAAGAAA ACGGTTTCCG GGGTAGGGGA GATCAGCTGG GAAGAAAGCA |
| 6501 | GGTTCCTGAG CAGCTGCGAC TTACCGCAGC CGGTGGGCCC GTAAATCACA |
| 6551 | CCTATTACCG GGTGCAACTG GTAGTTAAGA GAGCTGCAGC TGCCGTCATC |
| 6601 | CCTGAGCAGG GGGGCCACTT CGTTAAGCAT GTCCCTGACT CGCATGTTTT |
| 6651 | CCCTGACCAA ATCCGCCAGA AGGCGCTCGC CGCCCAGCGA TAGCAGTTCT |
| 6701 | TGCAAGGAAG CAAAGTTTTT CAACGGTTTG AGACCGTCCG CCGTAGGCAT |
| 6751 | GCTTTTGAGC GTTTGACCAA GCAGTTCCAG GCGGTCCCAC AGCTCGGTCA |
| 6801 | CCTGCTCTAC GGCATCTCGA TCCAGCATAT CTCCTCGTTT CGCGGGTTGG |
| 6851 | GGCGGCTTTC GCTGTACGGC AGTAGTCGGT GCTCGTCCAG ACGGGCCAGG |
| 6901 | GTCATGTCTT TCCACGGGCG CAGGGTCCTC GTCAGCGTAG TCTGGGTCAC |
| 6951 | GGTGAAGGGG TGCGCTCCGG GCTGCGCGCT GGCCAGGGTG CGCTTGAGGC |
| 7001 | TGGTCCTGCT GGTGCTGAAG CGCTGCCGGT CTTCGCCCTG CGCGTCGGCC |
| 7051 | AGGTAGCATT TGACCATGGT GTCATAGTCC AGCCCCTCCG CGGCGTGGCC |
| 7101 | CTTGGCGCGC AGCTTGCCCT TGGAGGAGGC GCCGCACGAG GGGCAGTGCA |
| 7151 | GACTTTTGAG GGCGTAGAGC TTGGGCGCGA GAAATACCGA TTCCGGGGAG |
| 7201 | TAGGCATCCG CGCCGCAGGC CCCGCAGACG GTCTCGCATT CCACGAGCCA |
| 7251 | GGTGAGCTCT GGCCGTTCGG GGTCAAAAAC CAGGTTTCCC CCATGCTTTT |
| 7301 | TGATGCGTTT CTTACCTCTG GTTTCCATGA GCCGGTGTCC ACGCTCGGTG |
| 7351 | ACGAAAAGGC TGTCCGTGTC CCCGTATACA GACTTGAGAG GCCTGTCCTC |
| 7401 | GAGCGGTGTT CCGCGGTCCT CCTCGTATAG AAACTCGGAC CACTCTGAGA |
| 7451 | CAAAGGCTCG CGTCCAGGCC AGCACGAAGG AGGCTAAGTG GGAGGGGTAG |
| 7501 | CGGTCGTTGT CCACTAGGGG GTCCACTCGC TCCAGGGTGT GAAGACACAT |
| 7551 | GTCGCCCTCT TCGGCATCAA GGAAGGTGAT TGGTTTGTAG GTGTAGGCCA |
| 7601 | CGTGACCGGG TGTTCCTGAA GGGGGGCTAT AAAAGGGGGT GGGGGCGCGT |
| 7651 | TCGTCCTCAC TCTCTTCCGC ATCGCTGTCT GCGAGGGCCA GCTGTTGGGG |
| 7701 | TGAGTACTCC CTCTGAAAAG CGGGCATGAC TTCTGCGCTA AGATTGTCAG |
| 7751 | TTTCCAAAAA CGAGGAGGAT TTGATATTCA CCTGGCCCGC GGTGATGCCT |
| 7801 | TTGAGGGTGG CCGCATCCAT CTGGTCAGAA AAGACAATCT TTTTGTTGTC |
| 7851 | AAGCTTGGTG GCAAACGACC CGTAGAGGGC GTTGGACAGC AACTTGGCGA |
| 7901 | TGGAGCGCAG GGTTTGGTTT TTGTCGCGAT CGGCGCGCTC CTTGGCCGCG |
| 7951 | ATGTTTAGCT GCACGTATTC GCGCGCAACG CACCGCCATT CGGGAAAGAC |
| 8001 | GGTGGTGCGC TCGTCGGGCA CCAGGTGCAC GCGCCAACCG CGGTTGTGCA |
| 8051 | GGGTGACAAG GTCAACGCTG GTGGCTACCT CTCCGCGTAG GCGCTCGTTG |
| 8101 | GTCCAGCAGA GGCGGCCGCC CTTGCGCGAG CAGAATGGCG GTAGGGGGTC |
| 8151 | TAGCTGCGTC TCGTCCGGGG GGTCTGCGTC CACGGTAAAG ACCCCGGGCA |
| 8201 | GCAGGCGCGC GTCGAAGTAG TCTATCTTGC ATCCTTGCAA GTCTAGCGCC |
| 8251 | TGCTGCCATG CGCGGGCGGC AAGCGCGCGC TCGTATGGGT TGAGTGGGGG |
| 8301 | ACCCCATGGC ATGGGGTGGG TGAGCGCGGA GGCGTACATG CCGCAAATGT |
| 8351 | CGTAAACGTA GAGGGGCTCT CTGAGTATTC CAAGATATGT AGGGTAGCAT |
| 8401 | CTTCCACCGC GGATGCTGGC GCGCACGTAA TCGTATAGTT CGTGCGAGGG |
| 8451 | AGCGAGGAGG TCGGGACCGA GGTTGCTACG GGCGGGCTGC TCTGCTCGGA |
| 8501 | AGACTATCTG CCTGAAGATG GCATGTGAGT TGGATGATAT GGTTGGACGC |
| 8551 | TGGAAGACGT TGAAGCTGGC GTCTGTGAGA CCTACCGCGT CACGCACGAA |
| 8601 | GGAGGCGTAG GAGTCGCGCA GCTTGTTGAC CAGCTCGGCG GTGACCTGCA |
| 8651 | CGTCTAGGGC GCAGTAGTCC AGGGTTTCCT TGATGATGTC ATACTTATCC |
| 8701 | TGTCCCTTTT TTTTCCACAG CTCGCGGTTG AGGACAAACT CTTCGCGGTC |
| 8751 | TTTCCAGTAC TCTTGGATCG GAAACCCGTC GGCCTCCGAA CGGTAAGAGC |
| 8801 | CTAGCATGTA GAACTGGTTG ACGGCCTGGT AGGCGCAGCA TCCCTTTTCT |
| 8851 | ACGGGTAGCG CGTATGCCTG CGCGGCCTTC CGGAGCGAGG TGTGGGTGAG |
| 8901 | CGCAAAGGTG TCCCTGACCA TGACTTTGAG GTACTGGTAT TTGAAGTCAG |
| 8951 | TGTCGTCGCA TCCGCCCTGC TCCCAGAGCA AAAAGTCCGT GCGCTTTTTG |
| 9001 | GAACGCGGAT TTGGCAGGGC GAAGGTGACA TCGTTGAAGA GTATCTTTCC |
| 9051 | CGCGCGAGGC ATAAAGTTGC GTGTGATGCG GAAGGGTCCC GGCACCTCGG |
| 9101 | AACGGTTGTT AATTACCTGG GCGGCGAGCA CGATCTCGTC AAAGCCGTTG |
| 9151 | ATGTTGTGGC CCACAATGTA AAGTTCCAAG AAGCGCGGGA TGCCCTTGAT |
| 9201 | GGAAGGCAAT TTTTTAAGTT CCTCGTAGGT GAGCTCTTCA GGGGAGCTGA |
| 9251 | GCCCGTGCTC TGAAAGGGCC CAGTCTGCAA GATGAGGGTT GGAAGCGACG |
| 9301 | AATGAGCTCC ACAGGTCACG GGCCATTAGC ATTTGCAGGT GGTCGCGAAA |
| 9351 | GGTCCTAAAC TGGCGACCTA TGGCCATTTT TTCTGGGGTG ATGCAGTAGA |
| 9401 | AGGTAAGCGG GTCTTGTTCC CAGCGGTCCC ATCCAAGGTT CGCGGCTAGG |
| 9451 | TCTCGCGCGG CAGTCACTAG AGGCTCATCT CCGCCGAACT TCATGACCAG |
| 9501 | CATGAAGGGC ACAGACTGCT TCCCAAAGGC CCCCATCCAA GTATAGGTCT |
| 9551 | CTACATCGTA GGTGACAAAG AGACGCTCGG TGCGAGGATG CGAGCCGATC |
| 9601 | GGGAAGAACT GGATCTCCCG CCACCAATTG GAGGAGTGGC TATTGATGTG |
| 9651 | GTGAAAGTAG AAGTCCCTGC GACGGGCCGA ACACTCGTGC TGGCTTTTGT |
| 9701 | AAAAACGTGC GCAGTACTGG CAGCGGTGCA CGGGCTGTAC ATCCTGCACG |
| 9751 | AGGTTGACCT GACGACCGCG CACAAGGAAG CAGAGTGGGA ATTTGAGCCC |
| 9801 | CTCGCCTGGC GGGTTTGGCT GGTGGTCTTC TACTTCGGCT GCTTGTCCTT |
| 9851 | GACCGTCTGG CTGCTCGAGG GGAGTTACGG TGGATCGGAC CACCACGCCG |
| 9901 | CGCGAGCCCA AAGTCCAGAT GTCCGCGCGC GGCGGTCGGA GCTTGATGAC |
| 9951 | AACATCGCGC AGATGGGAGC TGTCCATGGT CTGGAGCTCC CGCGGCGTCA |
| 10001 | GGTCAGGCGG GAGCTCCTGC AGGTTTACCT CGCATAGACG GGTCAGGGCG |
| 10051 | CGGGCTAGAT CCAGGTGATA CCTAATTTCC AGGGGCTGGT TGGTGGCGGC |
| 10101 | GTCGATGGCT TGCAAGAGGC CGCATCCCCG CGGCGCGACT ACGGTACCGC |
| 10151 | GCGGCGGGCG GTGGGCCGCG GGGGTGTCCT TGGATGATGC ATCTAAAAGC |
| 10201 | GGTGACGCGG GCGAGCCCCC GGAGGTAGGG GGGGCTCCGG ACCCGCCGGG |
| 10251 | AGAGGGGGCA GGGGCACGTC GGCGCCGCGC GCGGGCAGGA GCTGGTGCTG |
| 10301 | CGCGCGTAGG TTGCTGGCGA ACGCGACGAC GCGGCGGTTG ATCTCCTGAA |
| 10351 | TCTGGCGCCT CTGCGTGAAG ACGACGGGCC CGGTGAGCTT GAGCCTGAAA |
| 10401 | GAGAGTTCGA CAGAATCAAT TTCGGTGTCG TTGACGGCGG CCTGGCGCAA |
| 10451 | AATCTCCTGC ACGTCTCCTG AGTTGTCTTG ATAGGCGATC TCGGCCATGA |
| 10501 | ACTGCTCGAT CTCTTCCTCC TGGAGATCTC CGCGTCCGGC TCGCTCCACG |
| 10551 | GTGGCGGCGA GGTCGTTGGA AATGCGGGCC ATGAGCTGCG AGAAGGCGTT |
| 10601 | GAGGCCTCCC TCGTTCCAGA CGCGGCTGTA GACCACGCCC CCTTCGGCAT |
| 10651 | CGCGGGCGCG CATGACCACC TGCGCGAGAT TGAGCTCCAC GTGCCGGGCG |
| 10701 | AAGACGGCGT AGTTTCGCAG GCGCTGAAAG AGGTAGTTGA GGGTGGTGGC |
| 10751 | GGTGTGTTCT GCCACGAAGA AGTACATAAC CCAGCGTCGC AACGTGGATT |
| 10801 | CGTTGATATC CCCCAAGGCC TCAAGGCGCT CCATGGCCTC GTAGAAGTCC |
| 10851 | ACGGCGAAGT TGAAAAACTG GGAGTTGCGC GCCGACACGG TTAACTCCTC |
| 10901 | CTCCAGAAGA CGGATGAGCT CGGCGACAGT GTCGCGCACC TCGCGCTCAA |
| 10951 | AGGCTACAGG GGCCTCTTCT TCTTCTTCAA TCTCCTCTTC CATAAGGGCC |
| 11001 | TCCCCTTCTT CTTCTTCTGG CGGCGGTGGG GGAGGGGGGA CACGGCGGCG |
| 11051 | ACGACGGCGC ACCGGGAGGC GGTCGACAAA GCGCTCGATC ATCTCCCCGC |
| 11101 | GGCGACGGCG CATGGTCTCG GTGACGGCGC GGCCGTTCTC GCGGGGGCGC |
| 11151 | AGTTGGAAGA CGCCGCCCGT CATGTCCCGG TTATGGGTTG GCGGGGGGCT |
| 11201 | GCCATGCGGC AGGGATACGG CGCTAACGAT GCATCTCAAC AATTGTTGTG |
| 11251 | TAGGTACTCC GCCGCCGAGG GACCTGAGCG AGTCCGCATC GACCGGATCG |
| 11301 | GAAAACCTCT CGAGAAAGGC GTCTAACCAG TCACAGTCGC AAGGTAGGCT |
| 11351 | GAGCACCGTG GCGGGCGGCA GCGGGCGGCG GTCGGGGTTG TTTCTGGCGG |
| 11401 | AGGTGCTGCT GATGATGTAA TTAAAGTAGG CGGTCTTGAG ACGGCGGATG |
| 11451 | GTCGACAGAA GCACCATGTC CTTGGGTCCG GCCTGCTGAA TGCGCAGGCG |
| 11501 | GTCGGCCATG CCCCAGGCTT CGTTTTGACA TCGGCGCAGG TCTTTGTAGT |
| 11551 | AGTCTTGCAT GAGCCTTTCT ACCGGCACTT CTTCTTCTCC TTCCTCTTGT |
| 11601 | CCTGCATCTC TTGCATCTAT CGCTGCGGCG GCGGCGGAGT TTGGCCGTAG |
| 11651 | GTGGCGCCCT CTTCCTCCCA TGCGTGTGAC CCCGAAGCCC CTCATCGGCT |
| 11701 | GAAGCAGGGC TAGGTCGGCG ACAACGCGCT CGGCTAATAT GGCCTGCTGC |
| 11751 | ACCTGCGTGA GGGTAGACTG GAAGTCATCC ATGTCCACAA AGCGGTGGTA |
| 11801 | TGCGCCCGTG TTGATGGTGT AAGTGCAGTT GGCCATAACG GACCAGTTAA |
| 11851 | CGGTCTGGTG ACCCGGCTGC GAGAGCTCGG TGTACCTGAG ACGCGAGTAA |
| 11901 | GCCCTCGAGT CAAATACGTA GTCGTTGCAA GTCCGCACCA GGTACTGGTA |
| 11951 | TCCCACCAAA AAGTGCGGCG GCGGCTGGCG GTAGAGGGGC CAGCGTAGGG |
| 12001 | TGGCCGGGGC TCCGGGGGCG AGATCTTCCA ACATAAGGCG ATGATATCCG |
| 12051 | TAGATGTACC TGGACATCCA GGTGATGCCG GCGGCGGTGG TGGAGGCGCG |
| 12101 | CGGAAAGTCG CGGACGCGGT TCCAGATGTT GCGCAGCGGC AAAAAGTGCT |
| 12151 | CCATGGTCGG GACGCTCTGG CCGGTCAGGC GCGCGCAATC GTTGACGCTC |
| 12201 | TAGACCGTGC AAAAGGAGAG CCTGTAAGCG GGCACTCTTC CGTGGTCTGG |
| 12251 | TGGATAAATT CGCAAGGGTA TCATGGCGGA CGACCGGGGT TCGAGCCCCG |
| 12301 | TATCCGGCCG TCCGCCGTGA TCCATGCGGT TACCGCCCGC GTGTCGAACC |
| 12351 | CAGGTGTGCG ACGTCAGACA ACGGGGGAGT GCTCCTTTTG GCTTCCTTCC |
| 12401 | AGGCGCGGCG GCTGCTGCGC TAGCTTTTTT GGCCACTGGC CGCGCGCAGC |
| 12451 | GTAAGCGGTT AGGCTGGAAA GCGAAAGCAT TAAGTGGCTC GCTCCCTGTA |
| 12501 | GCCGGAGGGT TATTTTCCAA GGGTTGAGTC GCGGGACCCC CGGTTCGAGT |
| 12551 | CTCGGACCGG CCGGACTGCG GCGAACGGGG GTTTGCCTCC CCGTCATGCA |
| 12601 | AGACCCCGCT TGCAAATTCC TCCGGAAACA GGGACGAGCC CCTTTTTTGC |
| 12651 | TTTTCCCAGA TGCATCCGGT GCTGCGGCAG ATGCGCCCCC CTCCTCAGCA |
| 12701 | GCGGCAAGAG CAAGAGCAGC GGCAGACATG CAGGGCACCC TCCCCTCCTC |
| 12751 | CTACCGCGTC AGGAGGGGCG ACATCCGCGG TTGACGCGGC AGCAGATGGT |
| 12801 | GATTACGAAC CCCCGCGGCG CCGGGCCCGG CACTACCTGG ACTTGGAGGA |
| 12851 | GGGCGAGGGC CTGGCGCGGC TAGGAGCGCC CTCTCCTGAG CGGTACCCAA |
| 12901 | GGGTGCAGCT GAAGCGTGAT ACGCGTGAGG CGTACGTGCC GCGGCAGAAC |
| 12951 | CTGTTTCGCG ACCGCGAGGG AGAGGAGCCC GAGGAGATGC GGGATCGAAA |
| 13001 | GTTCCACGCA GGGCGCGAGC TGCGGCATGG CCTGAATCGC GAGCGGTTGC |
| 13051 | TGCGCGAGGA GGACTTTGAG CCCGACGCGC GAACCGGGAT TAGTCCCGCG |
| 13101 | CGCGCACACG TGGCGGCCGC CGACCTGGTA ACCGCATACG AGCAGACGGT |
| 13151 | GAACCAGGAG ATTAACTTTC AAAAAAGCTT TAACAACCAC GTGCGTACGC |
| 13201 | TTGTGGCGCG CGAGGAGGTG GCTATAGGAC TGATGCATCT GTGGGACTTT |
| 13251 | GTAAGCGCGC TGGAGCAAAA CCCAAATAGC AAGCCGCTCA TGGCGCAGCT |
| 13301 | GTTCCTTATA GTGCAGCACA GCAGGGACAA CGAGGCATTC AGGGATGCGC |
| 13351 | TGCTAAACAT AGTAGAGCCC GAGGGCCGCT GGCTGCTCGA TTTGATAAAC |
| 13401 | ATCCTGCAGA GCATAGTGGT GCAGGAGCGC AGCTTGAGCC TGGCTGACAA |
| 13451 | GGTGGCCGCC ATCAACTATT CCATGCTTAG CCTGGGCAAG TTTTACGCCC |
| 13501 | GCAAGATATA CCATACCCCT TACGTTCCCA TAGACAAGGA GGTAAAGATC |
| 13551 | GAGGGGTTCT ACATGCGCAT GGCGCTGAAG GTGCTTACCT TGAGCGACGA |
| 13601 | CCTGGGCGTT TATCGCAACG AGCGCATCCA CAAGGCCGTG AGCGTGAGCC |
| 13651 | GGCGGCGCGA GCTCAGCGAC CGCGAGCTGA TGCACAGCCT GCAAAGGGCC |
| 13701 | CTGGCTGGCA CGGGCAGCGG CGATAGAGAG GCCGAGTCCT ACTTTGACGC |
| 13751 | GGGCGCTGAC CTGCGCTGGG CCCCAAGCCG ACGCGCCCTG GAGGCAGCTG |
| 13801 | GGGCCGGACC TGGGCTGGCG GTGGCACCCG CGCGCGCTGG CAACGTCGGC |
| 13851 | GGCGTGGAGG AATATGACGA GGACGATGAG TACGAGCCAG AGGACGGCGA |
| 13901 | GTACTAAGCG GTGATGTTTC TGATCAGATG ATGCAAGACG CAACGGACCC |
| 13951 | GGCGGTGCGG GCGGCGCTGC AGAGCCAGCC GTCCGGCCTT AACTCCACGG |
| 14001 | ACGACTGGCG CCAGGTCATG GACCGCATCA TGTCGCTGAC TGCGCGCAAT |
| 14051 | CCTGACGCGT TCCGGCAGCA GCCGCAGGCC AACCGGCTCT CCGCAATTCT |
| 14101 | GGAAGCGGTG GTCCCGGCGC GCGCAAACCC CACGCACGAG AAGGTGCTGG |
| 14151 | CGATCGTAAA CGCGCTGGCC GAAAACAGGG CCATCCGGCC CGACGAGGCC |
| 14201 | GGCCTGGTCT ACGACGCGCT GCTTCAGCGC GTGGCTCGTT ACAACAGCGG |
| 14251 | CAACGTGCAG ACCAACCTGG ACCGGCTGGT GGGGGATGTG CGCGAGGCCG |
| 14301 | TGGCGCAGCG TGAGCGCGCG CAGCAGCAGG GCAACCTGGG CTCCATGGTT |
| 14351 | GCACTAAACG CCTTCCTGAG TACACAGCCC GCCAACGTGC CGCGGGGACA |
| 14401 | GGAGGACTAC ACCAACTTTG TGAGCGCACT GCGGCTAATG GTGACTGAGA |
| 14451 | CACCGCAAAG TGAGGTGTAC CAGTCTGGGC CAGACTATTT TTTCCAGACC |
| 14501 | AGTAGACAAG GCCTGCAGAC CGTAAACCTG AGCCAGGCTT TCAAAAACTT |
| 14551 | GCAGGGGCTG TGGGGGGTGC GGGCTCCCAC AGGCGACCGC GCGACCGTGT |
| 14601 | CTAGCTTGCT GACGCCCAAC TCGCGCCTGT TGCTGCTGCT AATAGCGCCC |
| 14651 | TTCACGGACA GTGGCAGCGT GTCCCGGGAC ACATACCTAG GTCACTTGCT |
| 14701 | GACACTGTAC CGCGAGGCCA TAGGTCAGGC GCATGTGGAC GAGCATACTT |
| 14751 | TCCAGGAGAT TACAAGTGTC AGCCGCGCGC TGGGGCAGGA GGACACGGGC |
| 14801 | AGCCTGGAGG CAACCCTAAA CTACCTGCTG ACCAACCGGC GGCAGAAGAT |
| 14851 | CCCCTCGTTG CACAGTTTAA ACAGCGAGGA GGAGCGCATT TTGCGCTACG |
| 14901 | TGCAGCAGAG CGTGAGCCTT AACCTGATGC GCGACGGGGT AACGCCCAGC |
| 14951 | GTGGCGCTGG ACATGACCGC GCGCAACATG GAACCGGGCA TGTATGCCTC |
| 15001 | AAACCGGCCG TTTATCAACC GCCTAATGGA CTACTTGCAT CGCGCGGCCG |
| 15051 | CCGTGAACCC CGAGTATTTC ACCAATGCCA TCTTGAACCC GCACTGGCTA |
| 15101 | CCGCCCCCTG GTTTCTACAC CGGGGGATTC GAGGTGCCCG AGGGTAACGA |
| 15151 | TGGATTCCTC TGGGACGACA TAGACGACAG CGTGTTTTCC CCGCAACCGC |
| 15201 | AGACCCTGCT AGAGTTGCAA CAGCGCGAGC AGGCAGAGGC GGCGCTGCGA |
| 15251 | AAGGAAAGCT TCCGCAGGCC AAGCAGCTTG TCCGATCTAG GCGCTGCGGC |
| 15301 | CCCGCGGTCA GATGCTAGTA GCCCATTTCC AAGCTTGATA GGGTCTCTTA |
| 15351 | CCAGCACTCG CACCACCCGC CCGCGCCTGC TGGGCGAGGA GGAGTACCTA |
| 15401 | AACAACTCGC TGCTGCAGCC GCAGCGCGAA AAAAACCTGC CTCCGGCATT |
| 15451 | TCCCAACAAC GGGATAGAGA GCCTAGTGGA CAAGATGAGT AGATGGAAGA |
| 15501 | CGTACGCGCA GGAGCACAGG GACGTGCCAG GCCCGCGCCC GCCCACCCGT |
| 15551 | CGTCAAAGGC ACGACCGTCA GCGGGGTCTG GTGTGGGAGG ACGATGACTC |
| 15601 | GGCAGACGAC AGCAGCGTCC TGGATTTGGG AGGGAGTGGC AACCCGTTTG |
| 15651 | CGCACCTTCG CCCCAGGCTG GGGAGAATGT TTTAAAAAAA AAAAAGCATG |
| 15701 | ATGCAAAATA AAAAACTCAC CAAGGCCATG GCACCGAGCG TTGGTTTTCT |
| 15751 | TGTATTCCCC TTAGTATGCG GCGCGCGGCG ATGTATGAGG AAGGTCCTCC |
| 15801 | TCCCTCCTAC GAGAGTGTGG TGAGCGCGGC GCCAGTGGCG GCGGCGCTGG |
| 15851 | GTTCTCCCTT CGATGCTCCC CTGGACCCGC CGTTTGTGCC TCCGCGGTAC |
| 15901 | CTGCGGCCTA CCGGGGGGAG AAACAGCATC CGTTACTCTG AGTTGGCACC |
| 15951 | CCTATTCGAC ACCACCCGTG TGTACCTGGT GGACAACAAG TCAACGGATG |
| 16001 | TGGCATCCCT GAACTACCAG AACGACCACA GCAACTTTCT GACCACGGTC |
| 16051 | ATTCAAAACA ATGACTACAG CCCGGGGGAG GCAAGCACAC AGACCATCAA |
| 16101 | TCTTGACGAC CGGTCGCACT GGGGCGGCGA CCTGAAAACC ATCCTGCATA |
| 16151 | CCAACATGCC AAATGTGAAC GAGTTCATGT TTACCAATAA GTTTAAGGCG |
| 16201 | CGGGTGATGG TGTCGCGCTT GCCTACTAAG GACAATCAGG TGGAGCTGAA |
| 16251 | ATACGAGTGG GTGGAGTTCA CGCTGCCCGA GGGCAACTAC TCCGAGACCA |
| 16301 | TGACCATAGA CCTTATGAAC AACGCGATCG TGGAGCACTA CTTGAAAGTG |
| 16351 | GGCAGACAGA ACGGGGTTCT GGAAAGCGAC ATCGGGGTAA AGTTTGACAC |
| 16401 | CCGCAACTTC AGACTGGGGT TTGACCCCGT CACTGGTCTT GTCATGCCTG |
| 16451 | GGGTATATAC AAACGAAGCC TTCCATCCAG ACATCATTTT GCTGCCAGGA |
| 16501 | TGCGGGGTGG ACTTCACCCA CAGCCGCCTG AGCAACTTGT TGGGCATCCG |
| 16551 | CAAGCGGCAA CCCTTCCAGG AGGGCTTTAG GATCACCTAC GATGATCTGG |
| 16601 | AGGGTGGTAA CATTCCCGCA CTGTTGGATG TGGACGCCTA CCAGGCGAGC |
| 16651 | TTGAAAGATG ACACCGAACA GGGCGGGGGT GGCGCAGGCG GCAGCAACAG |
| 16701 | CAGTGGCAGC GGCGCGGAAG AGAACTCCAA CGCGGCAGCC GCGGCAATGC |
| 16751 | AGCCGGTGGA GGACATGAAC GATCATGCCA TTCGCGGCGA CACCTTTGCC |
| 16801 | ACACGGGCTG AGGAGAAGCG CGCTGAGGCC GAAGCAGCGG CCGAAGCTGC |
| 16851 | CGCCCCCGCT GCGCAACCCG AGGTCGAGAA GCCTCAGAAG AAACCGGTGA |
| 16901 | TCAAACCCCT GACAGAGGAC AGCAAGAAAC GCAGTTACAA CCTAATAAGC |
| 16951 | AATGACAGCA CCTTCACCCA GTACCGCAGC TGGTACCTTG CATACAACTA |
| 17001 | CGGCGACCCT CAGACCGGAA TCCGCTCATG GACCCTGCTT TGCACTCCTG |
| 17051 | ACGTAACCTG CGGCTCGGAG CAGGTCTACT GGTCGTTGCC AGACATGATG |
| 17101 | CAAGACCCCG TGACCTTCCG CTCCACGCGC CAGATCAGCA ACTTTCCGGT |
| 17151 | GGTGGGCGCC GAGCTGTTGC CCGTGCACTC CAAGAGCTTC TACAACGACC |
| 17201 | AGGCCGTCTA CTCCCAACTC ATCCGCCAGT TTACCTCTCT GACCCACGTG |
| 17251 | TTCAATCGCT TTCCCGAGAA CCAGATTTTG GCGCGCCCGC CAGCCCCCAC |
| 17301 | CATCACCACC GTCAGTGAAA ACGTTCCTGC TCTCACAGAT CACGGGACGC |
| 17351 | TACCGCTGCG CAACAGCATC GGAGGAGTCC AGCGAGTGAC CATTACTGAC |
| 17401 | GCCAGACGCC GCACCTGCCC CTACGTTTAC AAGGCCCTGG GCATAGTCTC |
| 17451 | GCCGCGCGTC CTATCGAGCC GCACTTTTTG AGCAAGCATG TCCATCCTTA |
| 17501 | TATCGCCCAG CAATAACACA GGCTGGGGCC TGCGCTTCCC AAGCAAGATG |
| 17551 | TTTGGCGGGG CCAAGAAGCG CTCCGACCAA CACCCAGTGC GCGTGCGCGG |
| 17601 | GCACTACCGC GCGCCCTGGG GCGCGCACAA ACGCGGCCGC ACTGGGCGCA |
| 17651 | CCACCGTCGA TGACGCCATC GACGCGGTGG TGGAGGAGGC GCGCAACTAC |
| 17701 | ACGCCCACGC CGCCACCAGT GTCCACAGTG GACGCGGCCA TTCAGACCGT |
| 17751 | GGTGCGCGGA GCCCGGCGCT ATGCTAAAAT GAAGAGACGG CGGAGGCGCG |
| 17801 | TAGCACGTCG CCACCGCCGC CAGCCCGGCA CTGCCGCCCA ACGCGCGGCG |
| 17851 | GCGGCCCTGC TTAACCGCGC ACGTCGCACC GGCCGACGGG CGGCCATGCG |
| 17901 | GGCCGCTCGA AGGCTGGCCG CGGGTATTGT CACTGTGCCC CCCAGGTCCA |
| 17951 | GGCGACGAGC GGCCGCCGCA GCAGCCGCGG CCATTAGTGC TATGACTCAG |
| 18001 | GGTCGCAGGG GCAACGTGTA TTGGGTGCGC GACTCGGTTA GCGGCCTGCG |
| 18051 | CGTGCCCGTG CGCACCCGCC CCCCGCGCAA CTAGATTGCA AGAAAAAACT |
| 18101 | ACTTAGACTC GTACTGTTGT ATGTATCCAG CGGCGGCGGC GCGCAACGAA |
| 18151 | GCTATGTCCA AGCGCAAAAT CAAAGAAGAG ATGCTCCAGG TCATCGCGCC |
| 18201 | GGAGATCTAT GGCCCCCCGA AGAAGGAAGA GCAGGATTAC AAGCCCCGAA |
| 18251 | AGCTAAAGCG GGTCAAAAAG AAAAAGAAAG ATGATGATGA TGAACTTGAC |
| 18301 | GACGAGGTGG AACTGCTGCT CGCTACCGCG CCCAGGCGAC GGGTACAGTG |
| 18351 | GAAAGGTCGA CGCGTAAAAC GTGTTTTGCG ACCCGGCACC ACCGTAGTCT |
| 18401 | TTACGCCCGG TGAGCGCTCC ACCCGCACCT ACAAGCGCGT GTATGATGAG |
| 18451 | GTGTACGGCG ACGAGGACCT GCTTGAGCAG GCCAACGAGC GCCTCGGGGA |
| 18501 | GTTTGCCTAC GGAAAGCGGC ATAAGGACAT GCTGGCGTTG CCGCTGGACG |
| 18551 | AGGGCAACCC AACACCTAGC CTAAAGCCCG TAACACTGCA GCAGGTGCTG |
| 18601 | CCCGCGCTTG CACCGTCCGA AGAAAAGCGC GGCCTAAAGC GCGAGTCTGG |
| 18651 | TGACTTGGCA CCCACCGTGC AGCTGATGGT ACCCAAGCGC CAGCGACTGG |
| 18701 | AAGATGTCTT GGAAAAAATG ACCGTGGAAC CTGGGCTGGA GCCCGAGGTC |
| 18751 | CGCGTGCGGC CAATCAAGCA GGTGGCGCCG GGACTGGGCG TGCAGACCGT |
| 18801 | GGACGTTCAG ATACCCACTA CCAGTAGCAC CAGTATTGCC ACCGCCACAG |
| 18851 | AGGGCATGGA GACACAAACG TCCCCGGTTG CCTCAGCGGT GGCGGATGCC |
| 18901 | GCGGTGCAGG CGGTCGCTGC GGCCGCGTCC AAGACCTCTA CGGAGGTGCA |
| 18951 | AACGGACCCG TGGATGTTTC GCGTTTCAGC CCCCCGGCGC CCGCGCGGTT |
| 19001 | CGAGGAAGTA CGGCGCCGCC AGCGCGCTAC TGCCCGAATA TGCCCTACAT |
| 19051 | CCTTCCATTG CGCCTACCCC CGGCTATCGT GGCTACACCT ACCGCCCCAG |
| 19101 | AAGACGAGCA ACTACCCGAC GCCGAACCAC CACTGGAACC CGCCGCCGCC |
| 19151 | GTCGCCGTCG CCAGCCCGTG CTGGCCCCGA TTTCCGTGCG CAGGGTGGCT |
| 19201 | CGCGAAGGAG GCAGGACCCT GGTGCTGCCA ACAGCGCGCT ACCACCCCAG |
| 19251 | CATCGTTTAA AAGCCGGTCT TTGTGGTTCT TGCAGATATG GCCCTCACCT |
| 19301 | GCCGCCTCCG TTTCCCGGTG CCGGGATTCC GAGGAAGAAT GCACCGTAGG |
| 19351 | AGGGGCATGG CCGGCCACGG CCTGACGGGC GGCATGCGTC GTGCGCACCA |
| 19401 | CCGGCGGCGG CGCGCGTCGC ACCGTCGCAT GCGCGGCGGT ATCCTGCCCC |
| 19451 | TCCTTATTCC ACTGATCGCC GCGGCGATTG GCGCCGTGCC CGGAATTGCA |
| 19501 | TCCGTGGCCT TGCAGGCGCA GAGACACTGA TTAAAAACAA GTTGCATGTG |
| 19551 | GAAAAATCAA AATAAAAAGT CTGGACTCTC ACGCTCGCTT GGTCCTGTAA |
| 19601 | CTATTTTGTA GAATGGAAGA CATCAACTTT GCGTCTCTGG CCCCGCGACA |
| 19651 | CGGCTCGCGC CCGTTCATGG GAAACTGGCA AGATATCGGC ACCAGCAATA |
| 19701 | TGAGCGGTGG CGCCTTCAGC TGGGGCTCGC TGTGGAGCGG CATTAAAAAT |
| 19751 | TTCGGTTCCA CCGTTAAGAA CTATGGCAGC AAGGCCTGGA ACAGCAGCAC |
| 19801 | AGGCCAGATG CTGAGGGATA AGTTGAAAGA GCAAAATTTC CAACAAAAGG |
| 19851 | TGGTAGATGG CCTGGCCTCT GGCATTAGCG GGGTGGTGGA CCTGGCCAAC |
| 19901 | CAGGCAGTGC AAAATAAGAT TAACAGTAAG CTTGATCCCC GCCCTCCCGT |
| 19951 | AGAGGAGCCT CCACCGGCCG TGGAGACAGT GTCTCCAGAG GGGCGTGGCG |
| 20001 | AAAAGCGTCC GCGCCCCGAC AGGGAAGAAA CTCTGGTGAC GCAAATAGAC |
| 20051 | GAGCCTCCCT CGTACGAGGA GGCACTAAAG CAAGGCCTGC CCACCACCCG |
| 20101 | TCCCATCGCG CCCATGGCTA CCGGAGTGCT GGGCCAGCAC ACACCCGTAA |
| 20151 | CGCTGGACCT GCCTCCCCCC GCCGACACCC AGCAGAAACC TGTGCTGCCA |
| 20201 | GGCCCGACCG CCGTTGTTGT AACCCGTCCT AGCCGCGCGT CCCTGCGCCG |
| 20251 | CGCCGCCAGC GGTCCGCGAT CGTTGCGGCC CGTAGCCAGT GGCAACTGGC |
| 20301 | AAAGCACACT GAACAGCATC GTGGGTCTGG GGGTGCAATC CCTGAAGCGC |
| 20351 | CGACGATGCT TCTGAATAGC TAACGTGTCG TATGTGTGTC ATGTATGCGT |
| 20401 | CCATGTCGCC GCCAGAGGAG CTGCTGAGCC GCCGCGCGCC CGCTTTCCAA |
| 20451 | GATGGCTACC CCTTCGATGA TGCCGCAGTG GTCTTACATG CACATCTCGG |
| 20501 | GCCAGGACGC CTCGGAGTAC CTGAGCCCCG GGCTGGTGCA GTTTGCCCGC |
| 20551 | GCCACCGAGA CGTACTTCAG CCTGAATAAC AAGTTTAGAA ACCCCACGGT |
| 20601 | GGCGCCTACG CACGACGTGA CCACAGACCG GTCCCAGCGT TTGACGCTGC |
| 20651 | GGTTCATCCC TGTGGACCGT GAGGATACTG CGTACTCGTA CAAGGCGCGG |
| 20701 | TTCACCCTAG CTGTGGGTGA TAACCGTGTG CTGGACATGG CTTCCACGTA |
| 20751 | CTTTGACATC CGCGGCGTGC TGGACAGGGG CCCTACTTTT AAGCCCTACT |
| 20801 | CTGGCACTGC CTACAACGCC CTGGCTCCCA AGGGTGCCCC AAATCCTTGC |
| 20851 | GAATGGGATG AAGCTGCTAC TGCTCTTGAA ATAAACCTAG AAGAAGAGGA |
| 20901 | CGATGACAAC GAAGACGAAG TAGACGAGCA AGCTGAGCAG CAAAAAACTC |
| 20951 | ACGTATTTGG GCAGGCGCCT TATTCTGGTA TAAATATTAC AAAGGAGGGT |
| 21001 | ATTCAAATAG GTGTCGAAGG TCAAACACCT AAATATGCCG ATAAAACATT |
| 21051 | TCAACCTGAA CCTCAAATAG GAGAATCTCA GTGGTACGAA ACTGAAATTA |
| 21101 | ATCATGCAGC TGGGAGAGTC CTTAAAAAGA CTACCCCAAT GAAACCATGT |
| 21151 | TACGGTTCAT ATGCAAAACC CACAAATGAA AATGGAGGGC AAGGCATTCT |
| 21201 | TGTAAAGCAA CAAAATGGAA AGCTAGAAAG TCAAGTGGAA ATGCAATTTT |
| 21251 | TCTCAACTAC TGAGGCGACC GCAGGCAATG GTGATAACTT GACTCCTAAA |
| 21301 | GTGGTATTGT ACAGTGAAGA TGTAGATATA GAAACCCCAG ACACTCATAT |
| 21351 | TTCTTACATG CCCACTATTA AGGAAGGTAA CTCACGAGAA CTAATGGGCC |
| 21401 | AACAATCTAT GCCCAACAGG CCTAATTACA TTGCTTTTAG GGACAATTTT |
| 21451 | ATTGGTCTAA TGTATTACAA CAGCACGGGT AATATGGGTG TTCTGGCGGG |
| 21501 | CCAAGCATCG CAGTTGAATG CTGTTGTAGA TTTGCAAGAC AGAAACACAG |
| 21551 | AGCTTTCATA CCAGCTTTTG CTTGATTCCA TTGGTGATAG AACCAGGTAC |
| 21601 | TTTTCTATGT GGAATCAGGC TGTTGACAGC TATGATCCAG ATGTTAGAAT |
| 21651 | TATTGAAAAT CATGGAACTG AAGATGAACT TCCAAATTAC TGCTTTCCAC |
| 21701 | TGGGAGGTGT GATTAATACA GAGACTCTTA CCAAGGTAAA ACCTAAAACA |
| 21751 | GGTCAGGAAA ATGGATGGGA AAAAGATGCT ACAGAATTTT CAGATAAAAA |
| 21801 | TGAAATAAGA GTTGGAAATA ATTTTGCCAT GGAAATCAAT CTAAATGCCA |
| 21851 | ACCTGTGGAG AAATTTCCTG TACTCCAACA TAGCGCTGTA TTTGCCCGAC |
| 21901 | AAGCTAAAGT ACAGTCCTTC CAACGTAAAA ATTTCTGATA ACCCAAACAC |
| 21951 | CTACGACTAC ATGAACAAGC GAGTGGTGGC TCCCGGGTTA GTGGACTGCT |
| 22001 | ACATTAACCT TGGAGCACGC TGGTCCCTTG ACTATATGGA CAACGTCAAC |
| 22051 | CCATTTAACC ACCACCGCAA TGCTGGCCTG CGCTACCGCT CAATGTTGCT |
| 22101 | GGGCAATGGT CGCTATGTGC CCTTCCACAT CCAGGTGCCT CAGAAGTTCT |
| 22151 | TTGCCATTAA AAACCTCCTT CTCCTGCCGG GCTCATACAC CTACGAGTGG |
| 22201 | AACTTCAGGA AGGATGTTAA CATGGTTCTG CAGAGCTCCC TAGGAAATGA |
| 22251 | CCTAAGGGTT GACGGAGCCA GCATTAAGTT TGATAGCATT TGCCTTTACG |
| 22301 | CCACCTTCTT CCCCATGGCC CACAACACCG CCTCCACGCT TGAGGCCATG |
| 22351 | CTTAGAAACG ACACCAACGA CCAGTCCTTT AACGACTATC TCTCCGCCGC |
| 22401 | CAACATGCTC TACCCTATAC CCGCCAACGC TACCAACGTG CCCATATCCA |
| 22451 | TCCCCTCCCG CAACTGGGCG GCTTTCCGCG GCTGGGCCTT CACGCGCCTT |
| 22501 | AAGACTAAGG AAACCCCATC ACTGGGCTCG GGCTACGACC CTTATTACAC |
| 22551 | CTACTCTGGC TCTATACCCT ACCTAGATGG AACCTTTTAC CTCAACCACA |
| 22601 | CCTTTAAGAA GGTGGCCATT ACCTTTGACT CTTCTGTCAG CTGGCCTGGC |
| 22651 | AATGACCGCC TGCTTACCCC CAACGAGTTT GAAATTAAGC GCTCAGTTGA |
| 22701 | CGGGGAGGGT TACAACGTTG CCCAGTGTAA CATGACCAAA GACTGGTTCC |
| 22751 | TGGTACAAAT GCTAGCTAAC TACAACATTG GCTACCAGGG CTTCTATATC |
| 22801 | CCAGAGAGCT ACAAGGACCG CATGTACTCC TTCTTTAGAA ACTTCCAGCC |
| 22851 | CATGAGCCGT CAGGTGGTGG ATGATACTAA ATACAAGGAC TACCAACAGG |
| 22901 | TGGGCATCCT ACACCAACAC AACAACTCTG GATTTGTTGG CTACCTTGCC |
| 22951 | CCCACCATGC GCGAAGGACA GGCCTACCCT GCTAACTTCC CCTATCCGCT |
| 23001 | TATAGGCAAG ACCGCAGTTG ACAGCATTAC CCAGAAAAAG TTTCTTTGCG |
| 23051 | ATCGCACCCT TTGGCGCATC CCATTCTCCA GTAACTTTAT GTCCATGGGC |
| 23101 | GCACTCACAG ACCTGGGCCA AAACCTTCTC TACGCCAACT CCGCCCACGC |
| 23151 | GCTAGACATG ACTTTTGAGG TGGATCCCAT GGACGAGCCC ACCCTTCTTT |
| 23201 | ATGTTTTGTT TGAAGTCTTT GACGTGGTCC GTGTGCACCG GCCGCACCGC |
| 23251 | GGCGTCATCG AAACCGTGTA CCTGCGCACG CCCTTCTCGG CCGGCAACGC |
| 23301 | CACAACATAA AGAAGCAAGC AACATCAACA ACAGCTGCCG CCATGGGCTC |
| 23351 | CAGTGAGCAG GAACTGAAAG CCATTGTCAA AGATCTTGGT TGTGGGCCAT |
| 23401 | ATTTTTTGGG CACCTATGAC AAGCGCTTTC CAGGCTTTGT TTCTCCACAC |
| 23451 | AAGCTCGCCT GCGCCATAGT CAATACGGCC GGTCGCGAGA CTGGGGGCGT |
| 23501 | ACACTGGATG GCCTTTGCCT GGAACCCGCA CTCAAAAACA TGCTACCTCT |
| 23551 | TTGAGCCCTT TGGCTTTTCT GACCAGCGAC TCAAGCAGGT TTACCAGTTT |
| 23601 | GAGTACGAGT CACTCCTGCG CCGTAGCGCC ATTGCTTCTT CCCCCGACCG |
| 23651 | CTGTATAACG CTGGAAAAGT CCACCCAAAG CGTACAGGGG CCCAACTCGG |
| 23701 | CCGCCTGTGG ACTATTCTGC TGCATGTTTC TCCACGCCTT TGCCAACTGG |
| 23751 | CCCCAAACTC CCATGGATCA CAACCCCACC ATGAACCTTA TTACCGGGGT |
| 23801 | ACCCAACTCC ATGCTCAACA GTCCCCAGGT ACAGCCCACC CTGCGTCGCA |
| 23851 | ACCAGGAACA GCTCTACAGC TTCCTGGAGC GCCACTCGCC CTACTTCCGC |
| 23901 | AGCCACAGTG CGCAGATTAG GAGCGCCACT TCTTTTTGTC ACTTGAAAAA |
| 23951 | CATGTAAAAA TAATGTACTA GAGACACTTT CAATAAAGGC AAATGCTTTT |
| 24001 | ATTTGTACAC TCTCGGGTGA TTATTTACCC CCACCCTTGC CGTCTGCGCC |
| 24051 | GTTTAAAAAT CAAAGGGGTT CTGCCGCGCA TCGCTATGCG CCACTGGCAG |
| 24101 | GGACACGTTG CGATACTGGT GTTTAGTGCT CCACTTAAAC TCAGGCACAA |
| 24151 | CCATCCGCGG CAGCTCGGTG AAGTTTTCAC TCCACAGGCT GCGCACCATC |
| 24201 | ACCAACGCGT TTAGCAGGTC GGGCGCCGAT ATCTTGAAGT CGCAGTTGGG |
| 24251 | GCCTCCGCCC TGCGCGCGCG AGTTGCGATA CACAGGGTTG CAGCACTGGA |
| 24301 | ACACTATCAG CGCCGGGTGG TGCACGCTGG CCAGCACGCT CTTGTCGGAG |
| 24351 | ATCAGATCCG CGTCCAGGTC CTCCGCGTTG CTCAGGGCGA ACGGAGTCAA |
| 24401 | CTTTGGTAGC TGCCTTCCCA AAAAGGGCGC GTGCCCAGGC TTTGAGTTGC |
| 24451 | ACTCGCACCG TAGTGGCATC AAAAGGTGAC CGTGCCCGGT CTGGGCGTTA |
| 24501 | GGATACAGCG CCTGCATAAA AGCCTTGATC TGCTTAAAAG CCACCTGAGC |
| 24551 | CTTTGCGCCT TCAGAGAAGA ACATGCCGCA AGACTTGCCG GAAAACTGAT |
| 24601 | TGGCCGGACA GGCCGCGTCG TGCACGCAGC ACCTTGCGTC GGTGTTGGAG |
| 24651 | ATCTGCACCA CATTTCGGCC CCACCGGTTC TTCACGATCT TGGCCTTGCT |
| 24701 | AGACTGCTCC TTCAGCGCGC GCTGCCCGTT TTCGCTCGTC ACATCCATTT |
| 24751 | CAATCACGTG CTCCTTATTT ATCATAATGC TTCCGTGTAG ACACTTAAGC |
| 24801 | TCGCCTTCGA TCTCAGCGCA GCGGTGCAGC CACAACGCGC AGCCCGTGGG |
| 24851 | CTCGTGATGC TTGTAGGTCA CCTCTGCAAA CGACTGCAGG TACGCCTGCA |
| 24901 | GGAATCGCCC CATCATCGTC ACAAAGGTCT TGTTGCTGGT GAAGGTCAGC |
| 24951 | TGCAACCCGC GGTGCTCCTC GTTCAGCCAG GTCTTGCATA CGGCCGCCAG |
| 25001 | AGCTTCCACT TGGTCAGGCA GTAGTTTGAA GTTCGCCTTT AGATCGTTAT |
| 25051 | CCACGTGGTA CTTGTCCATC AGCGCGCGCG CAGCCTCCAT GCCCTTCTCC |
| 25101 | CACGCAGACA CGATCGGCAC ACTCAGCGGG TTCATCACCG TAATTTCACT |
| 25151 | TTCCGCTTCG CTGGGCTCTT CCTCTTCCTC TTGCGTCCGC ATACCACGCG |
| 25201 | CCACTGGGTC GTCTTCATTC AGCCGCCGCA CTGTGCGCTT ACCTCCTTTG |
| 25251 | CCATGCTTGA TTAGCACCGG TGGGTTGCTG AAACCCACCA TTTGTAGCGC |
| 25301 | CACATCTTCT CTTTCTTCCT CGCTGTCCAC GATTACCTCT GGTGATGGCG |
| 25351 | GGCGCTCGGG CTTGGGAGAA GGGCGCTTCT TTTTCTTCTT GGGCGCAATG |
| 25401 | GCCAAATCCG CCGCCGAGGT CGATGGCCGC GGGCTGGGTG TGCGCGGCAC |
| 25451 | CAGCGCGTCT TGTGATGAGT CTTCCTCGTC CTCGGACTCG ATACGCCGCC |
| 25501 | TCATCCGCTT TTTTGGGGGC GCCCGGGGAG GCGGCGGCGA CGGGGACGGG |
| 25551 | GACGACACGT CCTCCATGGT TGGGGGACGT CGCGCCGCAC CGCGTCCGCG |
| 25601 | CTCGGGGGTG GTTTCGCGCT GCTCCTCTTC CCGACTGGCC ATTTCCTTCT |
| 25651 | CCTATAGGCA GAAAAAGATC ATGGAGTCAG TCGAGAAGAA GGACAGCCTA |
| 25701 | ACCGCCCCCT CTGAGTTCGC CACCACCGCC TCCACCGATG CCGCCAACGC |
| 25751 | GCCTACCACC TTCCCCGTCG AGGCACCCCC GCTTGAGGAG GAGGAAGTGA |
| 25801 | TTATCGAGCA GGACCCAGGT TTTGTAAGCG AAGACGACGA GGACCGCTCA |
| 25851 | GTACCAACAG AGGATAAAAA GCAAGACCAG GACAACGCAG AGGCAAACGA |
| 25901 | GGAACAAGTC GGGCGGGGGG ACGAAAGGCA TGGCGACTAC CTAGATGTGG |
| 25951 | GAGACGACGT GCTGTTGAAG CATCTGCAGC GCCAGTGCGC CATTATCTGC |
| 26001 | GACGCGTTGC AAGAGCGCAG CGATGTGCCC CTCGCCATAG CGGATGTCAG |
| 26051 | CCTTGCCTAC GAACGCCACC TATTCTCACC GCGCGTACCC CCCAAACGCC |
| 26101 | AAGAAAACGG CACATGCGAG CCCAACCCGC GCCTCAACTT CTACCCCGTA |
| 26151 | TTTGCCGTGC CAGAGGTGCT TGCCACCTAT CACATCTTTT TCCAAAACTG |
| 26201 | CAAGATACCC CTATCCTGCC GTGCCAACCG CAGCCGAGCG GACAAGCAGC |
| 26251 | TGGCCTTGCG GCAGGGCGCT GTCATACCTG ATATCGCCTC GCTCAACGAA |
| 26301 | GTGCCAAAAA TCTTTGAGGG TCTTGGACGC GACGAGAAGC GCGCGGCAAA |
| 26351 | CGCTCTGCAA CAGGAAAACA GCGAAAATGA AAGTCACTCT GGAGTGTTGG |
| 26401 | TGGAACTCGA GGGTGACAAC GCGCGCCTAG CCGTACTAAA ACGCAGCATC |
| 26451 | GAGGTCACCC ACTTTGCCTA CCCGGCACTT AACCTACCCC CCAAGGTCAT |
| 26501 | GAGCACAGTC ATGAGTGAGC TGATCGTGCG CCGTGCGCAG CCCCTGGAGA |
| 26551 | GGGATGCAAA TTTGCAAGAA CAAACAGAGG AGGGCCTACC CGCAGTTGGC |
| 26601 | GACGAGCAGC TAGCGCGCTG GCTTCAAACG CGCGAGCCTG CCGACTTGGA |
| 26651 | GGAGCGACGC AAACTAATGA TGGCCGCAGT GCTCGTTACC GTGGAGCTTG |
| 26701 | AGTGCATGCA GCGGTTCTTT GCTGACCCGG AGATGCAGCG CAAGCTAGAG |
| 26751 | GAAACATTGC ACTACACCTT TCGACAGGGC TACGTACGCC AGGCCTGCAA |
| 26801 | GATCTCCAAC GTGGAGCTCT GCAACCTGGT CTCCTACCTT GGAATTTTGC |
| 26851 | ACGAAAACCG CCTTGGGCAA AACGTGCTTC ATTCCACGCT CAAGGGCGAG |
| 26901 | GCGCGCCGCG ACTACGTCCG CGACTGCGTT TACTTATTTC TATGCTACAC |
| 26951 | CTGGCAGACG GCCATGGGCG TTTGGCAGCA GTGCTTGGAG GAGTGCAACC |
| 27001 | TCAAGGAGCT GCAGAAACTG CTAAAGCAAA ACTTGAAGGA CCTATGGACG |
| 27051 | GCCTTCAACG AGCGCTCCGT GGCCGCGCAC CTGGCGGACA TCATTTTCCC |
| 27101 | CGAACGCCTG CTTAAAACCC TGCAACAGGG TCTGCCAGAC TTCACCAGTC |
| 27151 | AAAGCATGTT GCAGAACTTT AGGAACTTTA TCCTAGAGCG CTCAGGAATC |
| 27201 | TTGCCCGCCA CCTGCTGTGC ACTTCCTAGC GACTTTGTGC CCATTAAGTA |
| 27251 | CCGCGAATGC CCTCCGCCGC TTTGGGGCCA CTGCTACCTT CTGCAGCTAG |
| 27301 | CCAACTACCT TGCCTACCAC TCTGACATAA TGGAAGACGT GAGCGGTGAC |
| 27351 | GGTCTACTGG AGTGTCACTG TCGCTGCAAC CTATGCACCC CGCACCGCTC |
| 27401 | CCTGGTTTGC AATTCGCAGC TGCTTAACGA AAGTCAAATT ATCGGTACCT |
| 27451 | TTGAGCTGCA GGGTCCCTCG CCTGACGAAA AGTCCGCGGC TCCGGGGTTG |
| 27501 | AAACTCACTC CGGGGCTGTG GACGTCGGCT TACCTTCGCA AATTTGTACC |
| 27551 | TGAGGACTAC CACGCCCACG AGATTAGGTT CTACGAAGAC CAATCCCGCC |
| 27601 | CGCCAAATGC GGAGCTTACC GCCTGCGTCA TTACCCAGGG CCACATTCTT |
| 27651 | GGCCAATTGC AAGCCATCAA CAAAGCCCGC CAAGAGTTTC TGCTACGAAA |
| 27701 | GGGACGGGGG GTTTACTTGG ACCCCCAGTC CGGCGAGGAG CTCAACCCAA |
| 27751 | TCCCCCCGCC GCCGCAGCCC TATCAGCAGC AGCCGCGGGC CCTTGCTTCC |
| 27801 | CAGGATGGCA CCCAAAAAGA AGCTGCAGCT GCCGCCGCCA CCCACGGACG |
| 27851 | AGGAGGAATA CTGGGACAGT CAGGCAGAGG AGGTTTTGGA CGAGGAGGAG |
| 27901 | GAGGACATGA TGGAAGACTG GGAGAGCCTA GACGAGGAAG CTTCCGAGGT |
| 27951 | CGAAGAGGTG TCAGACGAAA CACCGTCACC CTCGGTCGCA TTCCCCTCGC |
| 28001 | CGGCGCCCCA GAAATCGGCA ACCGGTTCCA GCATGGCTAC AACCTCCGCT |
| 28051 | CCTCAGGCGC CGCCGGCACT GCCCGTTCGC CGACCCAACC GTAGATGGGA |
| 28101 | CACCACTGGA ACCAGGGCCG GTAAGTCCAA GCAGCCGCCG CCGTTAGCCC |
| 28151 | AAGAGCAACA ACAGCGCCAA GGCTACCGCT CATGGCGCGG GCACAAGAAC |
| 28201 | GCCATAGTTG CTTGCTTGCA AGACTGTGGG GGCAACATCT CCTTCGCCCG |
| 28251 | CCGCTTTCTT CTCTACCATC ACGGCGTGGC CTTCCCCCGT AACATCCTGC |
| 28301 | ATTACTACCG TCATCTCTAC AGCCCATACT GCACCGGCGG CAGCGGCAGC |
| 28351 | GGCAGCAACA GCAGCGGCCA CACAGAAGCA AAGGCGACCG GATAGCAAGA |
| 28401 | CTCTGACAAA GCCCAAGAAA TCCACAGCGG CGGCAGCAGC AGGAGGAGGA |
| 28451 | GCGCTGCGTC TGGCGCCCAA CGAACCCGTA TCGACCCGCG AGCTTAGAAA |
| 28501 | CAGGATTTTT CCCACTCTGT ATGCTATATT TCAACAGAGC AGGGGCCAAG |
| 28551 | AACAAGAGCT GAAAATAAAA AACAGGTCTC TGCGATCCCT CACCCGCAGC |
| 28601 | TGCCTGTATC ACAAAAGCGA AGATCAGCTT CGGCGCACGC TGGAAGACGC |
| 28651 | GGAGGCTCTC TTCAGTAAAT ACTGCGCGCT GACTCTTAAG GACTAGTTTC |
| 28701 | GCGCCCTTTC TCAAATTTAA GCGCGAAAAC TACGTCATCT CCAGCGGCCA |
| 28751 | CACCCGGCGC CAGCACCTGT CGTCAGCGCC ATTATGAGCA AGGAAATTCC |
| 28801 | CACGCCCTAC ATGTGGAGTT ACCAGCCACA AATGGGACTT GCGGCTGGAG |
| 28851 | CTGCCCAAGA CTACTCAACC CGAATAAACT ACATGAGCGC GGGACCCCAC |
| 28901 | ATGATATCCC GGGTCAACGG AATCCGCGCC CACCGAAACC GAATTCTCTT |
| 28951 | GGAACAGGCG GCTATTACCA CCACACCTCG TAATAACCTT AATCCCCGTA |
| 29001 | GTTGGCCCGC TGCCCTGGTG TACCAGGAAA GTCCCGCTCC CACCACTGTG |
| 29051 | GTACTTCCCA GAGACGCCCA GGCCGAAGTT CAGATGACTA ACTCAGGGGC |
| 29101 | GCAGCTTGCG GGCGGCTTTC GTCACAGGGT GCGGTCGCCC GGGCAGGGTA |
| 29151 | TAACTCACCT GACAATCAGA GGGCGAGGTA TTCAGCTCAA CGACGAGTCG |
| 29201 | GTGAGCTCCT CGCTTGGTCT CCGTCCGGAC GGGACATTTC AGATCGGCGG |
| 29251 | CGCCGGCCGT CCTTCATTCA CGCCTCGTCA GGCAATCCTA ACTCTGCAGA |
| 29301 | CCTCGTCCTC TGAGCCGCGC TCTGGAGGCA TTGGAACTCT GCAATTTATT |
| 29351 | GAGGAGTTTG TGCCATCGGT CTACTTTAAC CCCTTCTCGG GACCTCCCGG |
| 29401 | CCACTATCCG GATCAATTTA TTCCTAACTT TGACGCGGTA AAGGACTCGG |
| 29451 | CGGACGGCTA CGACTGATTA TTAAGTGGAG AGGCAGAGCA ACTGCGCCTG |
| 29501 | AAACACCTGG TCCACTGTCG CCGCCACAAG TGCTTTGCCC GCGACTCCGG |
| 29551 | TGAGTTTTGC TACTTTGAAT TGCCCGAGGA TCATATCGAG GATCTTTGTT |
| 29601 | GCCATCTCTG TGCTGAGTAT AATAAATACA GAAATTAAAA TATACTGGGG |
| 29651 | CTCCTATCGC CATCCTGTAA ACGCCACCGT CTTCACCCGC CCAAGCAAAC |
| 29701 | CAAGGCGAAC CTTACCTGGT ACTTTTAACA TCTCTCCCTC TGTGATTTAC |
| 29751 | AACAGTTTCA ACCCAGACGG AGTGAGTCTA CGAGAGAACC TCTCCGAGCT |
| 29801 | CAGCTACTCC ATCAGAAAAA ACACCACCCT CCTTACCTGC CGGGAACGTA |
| 29851 | CCCTTATATA AAAGTCAGGC TTCCTGGTGA TTAAATGAGA ATTTTAATTC |
| 29901 | GAATTTAAAT GAATTCGAGC TCGGTACCCG GGGATCGATC CGCCTCCGCA |
| 29951 | CCCGCCCTCA TGGAGGCCGT GGAGACCGGG GAACGGCCCA CCTTCGGAGC |
| 30001 | CTGGGACTAC GGGGTCTTTG CCCTCATGCT CCTGGTGTCC ACTGGCATCG |
| 30051 | GGCTGTGGGT CGGGCTGGCT CGGGGCGGGC AGCGCAGCGC TGAGGACTTC |
| 30101 | TTCACCGGGG GCCGGCGCCT GGCGGCCCTG CCCGTGGGCC TGTCGCTGTC |
| 30151 | TGCCAGCTTC ATGTCGGCCG TGCAGGTGCT GGGCGTGCCG TCGGAGGCCT |
| 30201 | ATCGCTATGG CCTCAAGTTC CTCTGGATGT GCCTGGGCCA GCTTCTGAAC |
| 30251 | TCGGTCCTCA CCGCCCTGCT CTTCATGCCC GTCTTCTACC GCCTGGGCCT |
| 30301 | CACCAGCACC TACGAGTACC TGGAGATGCG CTTCAGCCGC GCAGTGCGGC |
| 30351 | TCTGCGGGAC TTTGCAGTAC ATTGTAGCCA CGATGCTGTA CACCGGCATC |
| 30401 | GTAATCTACG CACCGGCCCT CATCCTGAAC CAAGTGACCG GGCTGGACAT |
| 30451 | CTGGGCGTCG CTCCTGTCCA CCGGAATTAT CTGCACCTTC TACACGGCTG |
| 30501 | TGGGCGGCAT GAAGGCTGTG GTCTGGACTG ATGTGTTCCA GGTCGTGGTA |
| 30551 | ATGCTAAGTG GCTTCTGGGT TGTCCTGGCA CGCGGTGTCA TGCTTGTGGG |
| 30601 | CGGGCCCCGN CAGGTGCTCA CGCTGGNCCA GAACCACTCC CGGATCAACC |
| 30651 | TCATGGACTT TAACCCTGAC CCGAGGAGCC GCTATNCATT CTGGACTTTT |
| 30701 | GTGGTGGGTG GCACGTTGGT GTGGCTCTCC ATGTATGGCG TGAACCANGC |
| 30751 | GCAGGTGCAG CGCTACNTGG CTTGCCGCAC AGAGAAGCAG GCCAAGCTGG |
| 30801 | CCCTGCTCAT CAACCAGGTC GGCCTGTTCC TGATCGTGTC CAGCGCTGCC |
| 30851 | TGCTGTGGCA TCGTCATGTT TGTGTTCTAC ACTGACTGCG ACCCTCTCCT |
| 30901 | CCTGGGGCGC ATCTCTGCCC CAGACCAGTA CATGCCTCTG CTGGTGCTGG |
| 30951 | ACATCTTCGA AGATCTGCCT GGAGTCCCCG GGCTTTTCCT GGCCTGTGCT |
| 31001 | TACAGTGGCA CCCTCAGCAC AGCATCCACC AGCATCAATG CTATGGCTGC |
| 31051 | AGTCACTGTA GAAGACCTCA TCAAACCTCG GCTGCGGAGC CTGGCACCCA |
| 31101 | GGAAACTCGT GATTATCTCC AAGGGGCTCT CACTCATCTA CGGATCGGCC |
| 31151 | TGTCTCACCG TGGCAGCCCT GTCCTCACTG CTCGGAGGAG GTGTCCTTCA |
| 31201 | GGGCTCCTTC ACCGTCATGG GAGTCATCAG CGGCCCCCTG CTGGGAGCCT |
| 31251 | TCATCTTGGG AATGTTCCTG CCGGCCTGCA ACACACCGGG CGTCCTCGCG |
| 30301 | GGACTAGGCG CGGGCTTGGC GCTGTCGCTG TGGGTGGCCT TGGGCGCCAC |
| 31351 | GCTGTACCCA CCCAGCGAGC AGACCATGAG GGTCCTGCCA TCGTCGGCTG |
| 31401 | CCCGCTGCGN GGCTCTCTCA GTCAACGCCT CTGGCCTCCT GGACCCGGCT |
| 31451 | CTCCTCCCTG CTAACGACTC CAGCAGGGCC CCCAGCTCAG GAATGGACGC |
| 31501 | CAGCCGACCC GCTTAGCTGA CAGCTTCTAT GCCATCTCCT ATCTCTATTA |
| 31551 | CGGTGCCCTG GGCACGCTGA CCACTGTGCT GTGCGGAGCC CTCATCAGCT |
| 31601 | GCCTGACAGG CCCCACCAAG CGCAGCACCC TGGCCCCGGG ATTGTTGTGG |
| 31651 | TGGGACCTCG CACGGCAGAC AGCATCAGTG GCCCCCAAGG AAGAAGTGGC |
| 31701 | CATCCTGGAT GACAACTTGG TCAAGGGTCC TGAAGAACTC CCCACTGGAA |
| 31751 | ACAAGAAGCC CCCTGGCTTC CTGCCCACCA ATGAGGATCG TCTGTTTTTC |
| 31801 | TTGGGGCAGA AGGAGCTGGA GGGGGCTGGC TCTTGGACCC CCTGTGTTGG |
| 31851 | ACATGATGGT GGTCGAGACC AGCAGGAGAC AAACCTCTGA GGACAGGGCC |
| 31901 | AGCCGCGGGA CTGACACCCT GGGATGGAGA ATTCTAGAGT CGACCTGCAG |
| 31951 | GAAATGGAAT TTCTGTCCAG TTTATTCAGC AGCACCTCCT TGCCCTCCTC |
| 32001 | CCAGCTCTGG TATTGCAGCT TCCTCCTGGC TGCAAACTTT CTCCACAATC |
| 32051 | TAAATGGAAT GTCAGTTTCC TCCTGTTCCT GTCCATCCGC ACCCACTATC |
| 32101 | TTCATGTTGT TGCAGATGAA GCGCGCAAGA CCGTCTGAAG ATACCTTCAA |
| 32151 | CCCCGTGTAT CCATATGACA CGGAAACCGG TCCTCCAACT GTGCCTTTTC |
| 32201 | TTACTCCTCC CTTTGTATCC CCCAATGGGT TTCAAGAGAG TCCCCCTGGG |
| 32251 | GTACTCTCTT TGCGCCTATC CGAACCTCTA GTTACCTCCA ATGGCATGCT |
| 32301 | TGCGCTCAAA ATGGGCAACG GCCTCTCTCT GGACGAGGCC GGCAACCTTA |
| 32351 | CCTCCCAAAA TGTAACCACT GTGAGCCCAC CTCTCAAAAA AACCAAGTCA |
| 32401 | AACATAAACC TGGAAATATC TGCACCCCTC ACAGTTACCT CAGAAGCCCT |
| 32451 | AACTGTGGCT GCCGCCGCAC CTCTAATGGT CGCGGGCAAC ACACTCACCA |
| 32501 | TGCAATCACA GGCCCCGCTA ACCGTGCACG ACTCCAAACT TAGCATTGCC |
| 32551 | ACCCAAGGAC CCCTCACAGT GTCAGAAGGA AAGCTAGCCC TGCAAACATC |
| 32601 | AGGCCCCCTC ACCACCACCG ATAGCAGTAC CCTTACTATC ACTGCCTCAC |
| 32651 | CCCCTCTAAC TACTGCCACT GGTAGCTTGG GCATTGACTT GAAAGAGCCC |
| 32701 | ATTTATACAC AAAATGGAAA ACTAGGACTA AAGTACGGGG CTCCTTTGCA |
| 32751 | TGTAACAGAC GACCTAAACA CTTTGACCGT AGCAACTGGT CCAGGTGTGA |
| 32801 | CTATTAATAA TACTTCCTTG CAAACTAAAG TTACTGGAGC CTTGGGTTTT |
| 32851 | GATTCACAAG GCAATATGCA ACTTAATGTA GCAGGAGGAC TAAGGATTGA |
| 32901 | TTCTCAAAAC AGACGCCTTA TACTTGATGT TAGTTATCCG TTTGATGCTC |
| 32951 | AAAACCAACT AAATCTAAGA CTAGGACAGG GCCCTCTTTT TATAAACTCA |
| 33001 | GCCCACAACT TGGATATTAA CTACAACAAA GGCCTTTACT TGTTTACAGC |
| 33051 | TTCAAACAAT TCCAAAAAGC TTGAGGTTAA CCTAAGCACT GCCAAGGGGT |
| 33101 | TGATGTTTGA CGCTACAGCC ATAGCCATTA ATGCAGGAGA TGGGCTTGAA |
| 33151 | TTTGGTTCAC CTAATGCACC AAACACAAAT CCCCTCAAAA CAAAAATTGG |
| 33201 | CCATGGCCTA GAATTTGATT CAAACAAGGC TATGGTTCCT AAACTAGGAA |
| 33251 | CTGGCCTTAG TTTTGACAGC ACAGGTGCCA TTACAGTAGG AAACAAAAAT |
| 33301 | AATGATAAGC TAACCCTATG GACAGGTCCA AAACCAGAAG CCAACTGCAT |
| 33351 | AATTGAATAC GGGAAACAAA ACCCAGATAG CAAACTAACT TTAATCCTTG |
| 33401 | TAAAAAATGG AGGAATTGTT AATGGATATG TAACGCTAAT GGGAGCCTCA |
| 33451 | GACTACGTTA ACACCTTATT TAAAAACAAA AATGTCTCCA TTAATGTAGA |
| 33501 | ACTATACTTT GATGCCACTG GTCATATATT ACCAGACTCA TCTTCTCTTA |
| 33551 | AAACAGATCT AGAACTAAAA TACAAGCAAA CCGCTGACTT TAGTGCAAGA |
| 33601 | GGTTTTATGC CAAGTACTAC AGCGTATCCA TTTGTCCTTC CTAATGCGGG |
| 33651 | AACACATAAT GAAAATTATA TTTTTGGTCA ATGCTACTAC AAAGCAAGCG |
| 33701 | ATGGTGCCCT TTTTCCGTTG GAAGTTACTG TTATGCTTAA TAAACGCCTG |
| 33751 | CCAGATAGTC GCACATCCTA TGTTATGACT TTTTTATGGT CCTTGAATGC |
| 33801 | TGGTCTAGCT CCAGAAACTA CTCAGGCAAC CCTCATAACC TCCCCATTTA |
| 33851 | CCTTTTCCTA TATTAGAGAA GATGACTAAT AAACTCTAAA GAATCGTTTG |
| 33901 | TGTTATGTTT CAACGTGTTT ATTTTTCAAT TGCAGAAAAT TTCAAGTCAT |
| 33951 | TTTTCATTCA GTAGTATAGC CCCACCACCA CATAGCTTAT ACAGATCACC |
| 34001 | GTACCTTAAT CAAACTCACA GAACCCTAGT ATTCAACCTG CCACCTCCCT |
| 34051 | CCCAACACAC AGAGTACACA GTCCTTTCTC CCCGGCTGGC CTTAAAAAGC |
| 34101 | ATCATATCAT GGGTAACAGA CATATTCTTA GGTGTTATAT TCCACACGGT |
| 34151 | TTCCTGTCGA GCCAAACGCT CATCAGTGAT ATTAATAAAC TCCCCGGGCA |
| 34201 | GCTCACTTAA GTTCATGTCG CTGTCCAGCT GCTGAGCCAC AGGCTGCTGT |
| 34251 | CCAACTTGCG GTTGCTTAAC GGGCGGCGAA GGAGAAGTCC ACGCCTACAT |
| 34301 | GGGGGTAGAG TCATAATCGT GCATCAGGAT AGGGCGGTGG TGCTGCAGCA |
| 34351 | GCGCGCGAAT AAACTGCTGC CGCCGCCGCT CCGTCCTGCA GGAATACAAC |
| 34401 | ATGGCAGTGG TCTCCTCAGC GATGATTCGC ACCGCCCGCA GCATAAGGCG |
| 34451 | CCTTGTCCTC CGGGCACAGC AGCGCACCCT GATCTCACTT AAATCAGCAC |
| 34501 | AGTAACTGCA GCACAGCACC ACAATATTGT TCAAAATCCC ACAGTGCAAG |
| 34551 | GCGCTGTATC CAAAGCTCAT GGCGGGGACC ACAGAACCCA CGTGGCCATC |
| 34601 | ATACCACAAG CGCAGGTAGA TTAAGTGGCG ACCCCTCATA AACACGCTGG |
| 34651 | ACATAAACAT TACCTCTTTT GGCATGTTGT AATTCACCAC CTCCCGGTAC |
| 34701 | CATATAAACC TCTGATTAAA CATGGCGCCA TCCACCACCA TCCTAAACCA |
| 34751 | GCTGGCCAAA ACCTGCCCGC CGGCTATACA CTGCAGGGAA CCGGGACTGG |
| 34801 | AACAATGACA GTGGAGAGCC CAGGACTCGT AACCATGGAT CATCATGCTC |
| 34851 | GTCATGATAT CAATGTTGGC ACAACACAGG CACACGTGCA TACACTTCCT |
| 34901 | CAGGATTACA AGCTCCTCCC GCGTTAGAAC CATATCCCAG GGAACAACCC |
| 34951 | ATTCCTGAAT CAGCGTAAAT CCCACACTGC AGGGAAGACC TCGCACGTAA |
| 35001 | CTCACGTTGT GCATTGTCAA AGTGTTACAT TCGGGCAGCA GCGGATGATC |
| 35051 | CTCCAGTATG GTAGCGCGGG TTTCTGTCTC AAAAGGAGGT AGACGATCCC |
| 35101 | TACTGTACGG AGTGCGCCGA GACAACCGAG ATCGTGTTGG TCGTAGTGTC |
| 35151 | ATGCCAAATG GAACGCCGGA CGTAGTCATA TTTCCTGAAG CAAAACCAGG |
| 35201 | TGCGGGCGTG ACAAACAGAT CTGCGTCTCC GGTCTCGCCG CTTAGATCGC |
| 35251 | TCTGTGTAGT AGTTGTAGTA TATCCACTCT CTCAAAGCAT CCAGGCGCCC |
| 35301 | CCTGGCTTCG GGTTCTATGT AAACTCCTTC ATGCGCCGCT GCCCTGATAA |
| 35351 | CATCCACCAC CGCAGAATAA GCCACACCCA GCCAACCTAC ACATTCGTTC |
| 35401 | TGCGAGTCAC ACACGGGAGG AGCGGGAAGA GCTGGAAGAA CCATGTTTTT |
| 35451 | TTTTTTATTC CAAAAGATTA TCCAAAACCT CAAAATGAAG ATCTATTAAG |
| 35501 | TGAACGCGCT CCCCTCCGGT GGCGTGGTCA AACTCTACAG CCAAAGAACA |
| 35551 | GATAATGGCA TTTGTAAGAT GTTGCACAAT GGCTTCCAAA AGGCAAACGG |
| 35601 | CCCTCACGTC CAAGTGGACG TAAAGGCTAA ACCCTTCAGG GTGAATCTCC |
| 35651 | TCTATAAACA TTCCAGCACC TTCAACCATG CCCAAATAAT TCTCATCTCG |
| 35701 | CCACCTTCTC AATATATCTC TAAGCAAATC CCGAATATTA AGTCCGGCCA |
| 35751 | TTGTAAAAAT CTGCTCCAGA GCGCCCTCCA CCTTCAGCCT CAAGCAGCGA |
| 35801 | ATCATGATTG CAAAAATTCA GGTTCCTCAC AGACCTGTAT AAGATTCAAA |
| 35851 | AGCGGAACAT TAACAAAAAT ACCGCGATCC CGTAGGTCCC TTCGCAGGGC |
| 35901 | CAGCTGAACA TAATCGTGCA GGTCTGCACG GACCAGCGCG GCCACTTCCC |
| 35951 | CGCCAGGAAC CATGACAAAA GAACCCACAC TGATTATGAC ACGCATACTC |
| 36001 | GGAGCTATGC TAACCAGCGT AGCCCCGATG TAAGCTTGTT GCATGGGCGG |
| 36051 | CGATATAAAA TGCAAGGTGC TGCTCAAAAA ATCAGGCAAA GCCTCGCGCA |
| 36101 | AAAAAGAAAG CACATCGTAG TCATGCTCAT GCAGATAAAG GCAGGTAAGC |
| 36151 | TCCGGAACCA CCACAGAAAA AGACACCATT TTTCTCTCAA ACATGTCTGC |
| 36201 | GGGTTTCTGC ATAAACACAA AATAAAATAA CAAAAAAACA TTTAAACATT |
| 36251 | AGAAGCCTGT CTTACAACAG GAAAAACAAC CCTTATAAGC ATAAGACGGA |
| 36301 | CTACGGCCAT GCCGGCGTGA CCGTAAAAAA ACTGGTCACC GTGATTAAAA |
| 36351 | AGCACCACCG ACAGCTCCTC GGTCATGTCC GGAGTCATAA TGTAAGACTC |
| 36401 | GGTAAACACA TCAGGTTGAT TCACATCGGT CAGTGCTAAA AAGCGACCGA |
| 36451 | AATAGCCCGG GGGAATACAT ACCCGCAGGC GTAGAGACAA CATTACAGCC |
| 36501 | CCCATAGGAG GTATAACAAA ATTAATAGGA GAGAAAAACA CATAAACACC |
| 36551 | TGAAAAACCC TCCTGCCTAG GCAAAATAGC ACCCTCCCGC TCCAGAACAA |
| 36601 | CATACAGCGC TTCCACAGCG GCAGCCATAA CAGTCAGCCT TACCAGTAAA |
| 36651 | AAAGAAAACC TATTAAAAAA ACACCACTCG ACACGGCACC AGCTCAATCA |
| 36701 | GTCACAGTGT AAAAAAGGGC CAAGTGCAGA GCGAGTATAT ATAGGACTAA |
| 36751 | AAAATGACGT AACGGTTAAA GTCCACAAAA AACACCCAGA AAACCGCACG |
| 36801 | CGAACCTACG CCCAGAAACG AAAGCCAAAA AACCCACAAC TTCCTCAAAT |
| 36851 | CGTCACTTCC GTTTTCCCAC GTTACGTCAC TTCCCATTTT AAGAAAACTA |
| 36901 | CAATTCCCAA CACATACAAG TTACTCCGCC CTAAAACCTA CGTCACCCGC |
| 36951 | CCCGTTCCCA CGCCCCGCGC CACGTCACAA ACTCCACCCC CTCATTATCA |
| 37001 | TATTGGCTTC AATCCAAAAT AAGGTATATT ATTGATGATG TTAATTAAGG |
| 37051 | ATCCNNNCGG TGTGAAATAC CGCACAGATG CGTAAGGAGA AAATACCGCA |
| 37101 | TCAGGCGCTC TTCCGCTTCC TCGCTCACTG ACTCGCTGCG CTCGGTCGTT |
| 37151 | CGGCTGCGGC GAGCGGTATC AGCTCACTCA AAGGCGGTAA TACGGTTATC |
| 37201 | CACAGAATCA GGGGATAACG CAGGAAAGAA CATGTGAGCA AAAGGCCAGC |
| 37251 | AAAAGGCCAG GAACCGTAAA AAGGCCGCGT TGCTGGCGTT TTTCCATAGG |
| 37301 | CTCCGCCCCC CTGACGAGCA TCACAAAAAT CGACGCTCAA GTCAGAGGTG |
| 37351 | GCGAAACCCG ACAGGACTAT AAAGATACCA GGCGTTTCCC CCTGGAAGCT |
| 37401 | CCCTCGTGCG CTCTCCTGTT CCGACCCTGC CGCTTACCGG ATACCTGTCC |
| 37451 | GCCTTTCTCC CTTCGGGAAG CGTGGCGCTT TCTCATAGCT CACGCTGTAG |
| 37501 | GTATCTCAGT TCGGTGTAGG TCGTTCGCTC CAAGCTGGGC TGTGTGCACG |
| 37551 | AACCCCCCGT TCAGCCCGAC CGCTGCGCCT TATCCGGTAA CTATCGTCTT |
| 37601 | GAGTCCAACC CGGTAAGACA CGACTTATCG CCACTGGCAG CAGCCACTGG |
| 37651 | TAACAGGATT AGCAGAGCGA GGTATGTAGG CGGTGCTACA GAGTTCTTGA |
| 37701 | AGTGGTGGCC TAACTACGGC TACACTAGAA GGACAGTATT TGGTATCTGC |
| 37751 | GCTCTGCTGA AGCCAGTTAC CTTCGGAAAA AGAGTTGGTA GCTCTTGATC |
| 37801 | CGGCAAACAA ACCACCGCTG GTAGCGGTGG TTTTTTTGTT TGCAAGCAGC |
| 37851 | AGATTACGCG CAGAAAAAAA GGATCTCAAG AAGATCCTTT GATCTTTTCT |
| 37901 | ACGGGGTCTG ACGCTCAGTG GAACGAAAAC TCACGTTAAG GGATTTTGGT |
| 37951 | CATGAGATTA TCAAAAAGGA TCTTCACCTA GATCCTTTTA AATTAAAAAT |
| 38001 | GAAGTTTTAA ATCAATCTAA AGTATATATG AGTAAACTTG GTCTGACAGT |
| 38051 | TACCAATGCT TAATCAGTGA GGCACCTATC TCAGCGATCT GTCTATTTCG |
| 38101 | TTCATCCATA GTTGCCTGAC TCCCCGTCGT GTAGATAACT ACGATACGGG |
| 38151 | AGGGCTTACC ATCTGGCCCC AGTGCTGCAA TGATACCGCG AGACCCACGC |
| 38201 | TCACCGGCTC CAGATTTATC AGCAATAAAC CAGCCAGCCG GAAGGGCCGA |
| 38251 | GCGCAGAAGT GGTCCTGCAA CTTTATCCGC CTCCATCCAG TCTATTAATT |
| 38301 | GTTGCCGGGA AGCTAGAGTA AGTAGTTCGC CAGTTAATAG TTTGCGCAAC |
| 38351 | GTTGTTGNNN NNNAAAAAGG ATCTTCACCT AGATCCTTTT CACGTAGAAA |
| 38401 | GCCAGTCCGC AGAAACGGTG CTGACCCCGG ATGAATGTCA GCTACTGGGC |
| 38451 | TATCTGGACA AGGGAAAACG CAAGCGCAAA GAGAAAGCAG GTAGCTTGCA |
| 38501 | GTGGGCTTAC ATGGCGATAG CTAGACTGGG CGGTTTTATG GACAGCAAGC |
| 38551 | GAACCGGAAT TGCCAGCTGG GGCGCCCTCT GGTAAGGTTG GGAAGCCCTG |
| 38601 | CAAAGTAAAC TGGATGGCTT TCTCGCCGCC AAGGATCTGA TGGCGCAGGG |
| 38651 | GATCAAGCTC TGATCAAGAG ACAGGATGAG GATCGTTTCG CATGATTGAA |
| 38701 | CAAGATGGAT TGCACGCAGG TTCTCCGGCC GCTTGGGTGG AGAGGCTATT |
| 38751 | CGGCTATGAC TGGGCACAAC AGACAATCGG CTGCTCTGAT GCCGCCGTGT |
| 38801 | TCCGGCTGTC AGCGCAGGGG CGCCCGGTTC TTTTTGTCAA GACCGACCTG |
| 38851 | TCCGGTGCCC TGAATGAACT GCAAGACGAG GCAGCGCGGC TATCGTGGCT |
| 38901 | GGCCACGACG GGCGTTCCTT GCGCAGCTGT GCTCGACGTT GTCACTGAAG |
| 38951 | CGGGAAGGGA CTGGCTGCTA TTGGGCGAAG TGCCGGGGCA GGATCTCCTG |
| 39001 | TCATCTCACC TTGCTCCTGC CGAGAAAGTA TCCATCATGG CTGATGCAAT |
| 39051 | GCGGCGGCTG CATACGCTTG ATCCGGCTAC CTGCCCATTC GACCACCAAG |
| 39101 | CGAAACATCG CATCGAGCGA GCACGTACTC GGATGGAAGC CGGTCTTGTC |
| 39151 | GATCAGGATG ATCTGGACGA AGAGCATCAG GGGCTCGCGC CAGCCGAACT |
| 39201 | GTTCGCCAGG CTCAAGGCGA GCATGCCCGA CGGCGAGGAT CTCGTCGTGA |
| 39251 | CCCATGGCGA TGCCTGCTTG CCGAATATCA TGGTGGAAAA TGGCCGCTTT |
| 39301 | TCTGGATTCA TCGACTGTGG CCGGCTGGGT GTGGCGGACC GCTATCAGGA |
| 39351 | CATAGCGTTG GCTACCCGTG ATATTGCTGA AGAGCTTGGC GGCGAATGGG |
| 39401 | CTGACCGCTT CCTCGTGCTT TACGGTATCG CCGCTCCCGA TTCGCAGCGC |
| 39451 | ATCGCCTTCT ATCGCCTTCT TGACGAGTTC TTCTGAATTT TGTTAAAATT |
| 39501 | TTTGTTAAAT CAGCTCATTT TTTAACCAAT AGGCCGAAAT CGGCAACATC |
| 39551 | CCTTATAAAT CAAAAGAATA GACCGCGATA GGGTTGAGTG TTGTTCCAGT |
| 39601 | TTGGAACAAG AGTCCACTAT TAAAGAACGT GGACTCCAAC GTCAAAGGGC |
| 39651 | GAAAAACCGT CTATCAGGGC GATGGCCCAC TACGTGAACC ATCACCCAAA |
| 39701 | TCAAGTTTTT TGCGGTCGAG GTGCCGTAAA GCTCTAAATC GGAACCCTAA |
| 39751 | AGGGAGCCCC CGATTTAGAG CTTGACGGGG AAAGCCGGCG AACGTGGCGA |
| 39801 | GAAAGGAAGG GAAGAAAGCG AAAGGAGCGG GCGCTAGGGC GCTGGCAAGT |
| 39851 | GTAGCGGTCA CGCTGCGCGT AACCACCACA CCCGCGCGCT TAATGCGCCG |
| 39901 | CTACAGGGCG CGTCCATTCG CCATTCAGGA TCGAATTAAT TCTTAA |

What is claimed is:

1. A genetically-modified adenovirus construct comprising:
at least a partial deletion of E3;
a heterologous polynucleotide encoding a heterologous polypeptide in place of the at least partial deletion of E3; and
a cell-specific regulatory polynucleotide operationally linked to the heterologous polynucleotide.

2. The adenovirus construct of claim 1 wherein the heterologous polynucleotide encodes a therapeutic polypeptide effective for treating cancer.

3. The adenovirus construct of claim 2 wherein the therapeutic polypeptide comprises a polypeptide that kills tumor cells.

4. The adenovirus construct of claim 2 wherein the therapeutic polypeptide comprises a polypeptide that sensitizes tumor cells to a primary cancer therapy.

5. The adenovirus construct of claim 4 wherein the primary cancer therapy comprises radiotherapy.

6. The adenovirus construct of claim 4 wherein the primary cancer therapy comprises chemotherapy.

7. The adenovirus construct of claim 4 wherein the primary cancer therapy comprises immunotherapy.

8. The adenovirus construct of claim 1 wherein the heterologous polynucleotide encodes a diagnostic polypeptide.

9. The adenovirus construct of claim 8 wherein the diagnostic polypeptide binds to a radioisotope.

10. The adenovirus construct of claim 1 wherein the cell-specific regulatory polynucleotide comprises a tumor-specific promoter.

11. The adenovirus construct of claim 1 further comprising a genetic modification that increases transduction efficiency of the adenovirus construct into target cells.

12. The adenovirus construct of claim 11 wherein the genetic modification that increases transduction efficiency comprises a polynucleotide that encodes a fiber knob polypeptide of adenovirus type 3 (Ad3).

13. The adenovirus construct of claim 1 wherein the at least partial deletion of E3 comprises a deletion of at least a portion of the Adenovirus Death Protein (ADP) coding region.

14. The adenovirus construct of claim 1 wherein the construct includes the Adenovirus Death Protein (ADP) coding region.

15. A method of visualizing a cell, the method comprising:
introducing into a target cell a genetically-modified adenovirus construct comprising:
at least a partial deletion of E3;
a heterologous polynucleotide encoding a heterologous polypeptide in place of the at least partial deletion of E3, the heterologous polypeptide capable of generating a detectable signal; and
a regulatory polynucleotide operationally linked to the heterologous polynucleotide that initiates expression of the heterologous polypeptide when the adenovirus construct is introduced into the target cell; and
visualizing the detectable signal.

16. The method of claim 15 wherein:
the heterologous polypeptide binds a radioisotope; and
the method further includes contacting the cell with the radioisotope.

17. The method of claim 15 wherein the regulatory polynucleotide does not initiate expression of the heterologous polypeptide when the adenovirus construct is introduced into a cell other than the target cell.

18. The method of claim 15 wherein the at least partial deletion of E3 comprises a deletion of at least a portion of the Adenovirus Death Protein (ADP) coding region.

19. A method of treating a condition in a subject, the method comprising:
administering to the subject a composition that includes a genetically-modified adenovirus construct, the adenovirus construct comprising:
at least a partial deletion of E3;
a heterologous polynucleotide encoding a therapeutic polypeptide in place of the at least partial deletion of E3; and
a cell-specific regulatory polynucleotide operationally linked to the therapeutic polynucleotide.

20. The method of claim 19 wherein:
the condition comprises cancer; and
the cell-specific regulatory polynucleotide comprises a tumor-specific promoter.

21. The method of claim 19 wherein the therapeutic polypeptide comprises a cytotoxic polypeptide.

22. The method of claim 19 wherein the therapeutic polypeptide comprises a polypeptide that sensitizes tumor cells to an anti-tumor therapy.

23. The method of claim 19 wherein the at least partial deletion of E3 comprises a deletion of at least a portion of the Adenovirus Death Protein (ADP) coding region.

24. The method of claim 19 wherein the construct includes the Adenovirus Death Protein (ADP) coding region.


SEQUENCE LISTING (2 sequences)

SEQ ID NO: 1 - DNA, 35721 bp, artificial
  Polynucleotide
  misc_feature (34950)..(34950): n is a, c, g, or t
catcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatatttgtctagggccgcggggactttgaccgtttacgtggagactcgcccaggtgtttttctcaggtgttttccgcgttccgggtcaaagttggcgttttattattatagtcagctgacgtgtagtgtatttatacccggtgagttcctcaagaggccactcttgagtgccagcgagtagagttttctcctccgagccgctccgacaccgggactgaaaatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaaacctgtgattgcgtgtgtggttaacgcctttgtttgctgaatgagttgatgtaagtttaataaagggtgagataatgtttaacttgcatggcgtgttaaatggggcggggcttaaagggtatataatgcgccgtgggctaatcttggttacatctgacctcatggaggcttgggagtgtttggaagatttttctgctgtgcgtaacttgctggaacagagctctaacagtacctcttggttttggaggtttctgtggggctcatcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtacaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtaaggtttactggccccaattttagcggtacggttttcctggccaataccaaccttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctctttgaaaggtgtaccttgggtatcctgtctgagggtaactccagggtgcgccacaatgtggcctccgactgtggttgcttcatgctagtgaaaagcgtggctgtgattaagcataacatggtatgtggcaactgcgaggacagggcctctcagatgctgacctgctcggacggcaactgtcacctgctgaagaccattcacgtagccagccactctcgcaaggcctggccagtgtttgagcataacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgcggttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgaatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgtccttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgattattaagtggagaggcagagcaactgcgcctgaaacacctggtccactgtcgccgccacaagtgctttgcccgcgactccggtgagttttgctactttgaattgcccgaggatcatatcgaggatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacccttatataaaagtcaggcttcctggtgattaaatgagaattttaattcgaatttaaatgaattcgagctcggtacccggggatcctctagcccgggctcgagatctgcgatctaagtaagctctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgacctgcaggaaatggaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaactttgtggaccacaccagctccatctcctaactgtagactaaatgcagagaaagatgctaaactcactttggtcttaacaaaatgtggcagtcaaatacttgctacagtttcagttttggctgttaaaggcagtttggctccaatatctggaacagttcaaagtgctcatcttattataagatttgacgaaaatggagtgctactaaacaattccttcctggacccagaatattggaactttagaaatggagatcttactgaaggcacagcctatacaaacgctgttggatttatgcctaacctatcagcttatccaaaatctcacggtaaaactgccaaaagtaacattgtcagtcaagtttacttaaacggagacaaaactaaacctgtaacactaaccattacactaaacggtacacaggaaacaggagacacaactccaagtgcatactctatgtcattttcatgggactggtctggccacaactacattaatgaaatatttgccacatcctcttacactttttcatacattgcccaagaataaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttaattaagaattcggatctgcgacgcgaggctggatggccttccccattatgattcttctcgcttccggcggcatcgggatgcccgcgttgcaggccatgctgtccaggcaggtagatgacgaccatcagggacagcttcacggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgntgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtcttcaaggatccgaattcttaattaa

SEQ ID NO: 2 - DNA, 39946 bp, artificial
  Polynucleotide
  misc_feature (30610)..(30610): n is a, c, g, or t
  misc_feature (30627)..(30627): n is a, c, g, or t
  misc_feature (30686)..(30686): n is a, c, g, or t
  misc_feature (30748)..(30748): n is a, c, g, or t
  misc_feature (30767)..(30767): n is a, c, g, or t
  misc_feature (31410)..(31410): n is a, c, g, or t
  misc_feature (37055)..(37057): n is a, c, g, or t
  misc_feature (38358)..(38363): n is a, c, g, or t
ttaacatcatcaataatataccttattttggattgaagccaatatgataatgagggggtggagtttgtgacgtggcgcggggcgtgggaacggggcgggtgacgtagtagtgtggcggaagtgtgatgttgcaagtgtggcggaacacatgtaagcgacggatgtggcaaaagtgacgtttttggtgtgcgccggtgtacacaggaagtgacaattttcgcgcggttttaggcggatgttgtagtaaatttgggcgtaaccgagtaagatttggccattttcgcgggaaaactgaataagaggaagtgaaatctgaataattttgtgttactcatagcgcgtaatactggtacggggtcgaccccgtacctggtgtagttttatttcaggttttatactgtcattttcctgtaatgctaaggacttaggacataactgaattttctattttccacttcttttctggtgtgtgtgtatatatatatgtatatatacacacacacatatacatatatatattttttagtatctcaccctcacatgctcctccctgagcactacccatgatagatgttaaacaaaagcaaagatgaaattccaactgtcaaaatctcccttccatctaattaattcctcatccaactatgttccaaaacgagaatagaaaattagccccaataagcccaggcaactgaaaagtaaatgctatgttgtactttgatccatggtcacaactcataatcttggaaaagtggacagaaaagacaaaagagtgaactttaaaactcgaatttattttaccagtatctcctatgaagggctagtaaccaaaataatccacgcatcagggagagaaatgccttaaggcatacgttttggacatttagcgtccctgcaaattctggccatcgccgcttcctttgtccatcagaaggcaggaaactttatattggtgacccgtggagctcacattaactatttacagggtaactgcttaggaccagtattatgaggagaatttacctttcccgcctctctttccaagaaacaaggagggggtgaaggtacggagaacagtatttcttctgttgaaagcaacttagctacaaagataaattacagctatgtacactgaaggtagctatttcattccacaaaataagagttttttaaaaagctatgtatgtatgtcctgcatatagagcagatatacagcctattaagcgtcgtcactaaaacataaaacatgtcagcctttcttaaccttactcgccccagtctgtcccgacgtgacttcctcgaccctctaaagacgtacagaccagacacggcggcggtggcgggagaggggattccctgcgcccccggacctcagggccgctcagattcctggagaggaagccaagtgtccttctgccctcccccggtatcccatccaaggcgatcagtccagaactggctctcggaagcgctcgggcaaagactgcgaagaagaaaagacatctggcggaaacctgtgcgcctggggcggtggaactcggggaggagagggagggatcagacaggagagtggggactaccccctctgctcccaaattggggcagcttcctgggtttccgattttctcatttccgtgggtaaaaaaccctgcccccaccgggcttacgcaatttttttaaggggagaggagggaaaaatttgtggggggtacgaaaaggcggaaagaaacagtcatttcgtcacatgggcttggttttcagtcttataaaaaggaaggttctctcggttagcgaccaattgtcatacgacttgcagtgagcgtcaggagcacgtccaggaactcctcagcagggctgcaggaattcgatatcaagcttgaaaatgagacatattatctgccacggaggtgttattaccgaagaaatggccgccagtcttttggaccagctgatcgaagaggtactggctgataatcttccacctcctagccattttgaaccacctacccttcacgaactgtatgatttagacgtgacggcccccgaagatcccaacgaggaggcggtttcgcagatttttcccgactctgtaatgttggcggtgcaggaagggattgacttactcacttttccgccggcgcccggttctccggagccgcctcacctttcccggcagcccgagcagccggagcagagagccttgggtccggtttctatgccaaaccttgtaccggaggtgatcgatcttacctgccacgaggctggctttccacccagtgacgacgaggatgaagagggtgaggagtttgtgttagattatgtggagcaccccgggcacggttgcaggtcttgtcattatcaccggaggaatacgggggacccagatattatgtgttcgctttgctatatgaggacctgtggcatgtttgtctacagtaagtgaaaattatgggcagtgggtgatagagtggtgggtttggtgtggtaattttttttttaatttttacagttttgtggtttaaagaattttgtattgtgatttttttaaaaggtcctgtgtctgaacctgagcctgagcccgagccagaaccggagcctgcaagacctacccgccgtcctaaaatggcgcctgctatcctgagacgcccgacatcacctgtgtctagagaatgcaatagtagtacggatagctgtgactccggtccttctaacacacctcctgagatacacccggtggtcccgctgtgccccattaaaccagttgccgtgagagttggtgggcgtcgccaggctgtggaatgtatcgaggacttgcttaacgagcctgggcaacctttggacttgagctgtaaacgccccaggccataaggtgtaaacctgtgattgcgtgtgtggttaacgcctttgtttgctgaatgagttgatgtaagtttaataaagggtgagataatgtttaacttgcatggcgtgttaaatggggcggggcttaaagggtatataatgcgccgtgggctaatcttggttacatctgacctcatggaggcttgggagtgtttggaagatttttctgctgtgcgtaacttgctggaacagagctctaacagtacctcttggttttggaggtttctgtggggctcatcccaggcaaagttagtctgcagaattaaggaggattacaagtgggaatttgaagagcttttgaaatcctgtggtgagctgtttgattctttgaatctgggtcaccaggcgcttttccaagagaaggtcatcaagactttggatttttccacaccggggcgcgctgcggctgctgttgcttttttgagttttataaaggataaatggagcgaagaaacccatctgagcggggggtacctgctggattttctggccatgcatctgtggagagcggttgtgagacacaagaatcgcctgctactgttgtcttccgtccgcccggcgataataccgacggaggagcagcagcagcagcaggaggaagccaggcggcggcggcaggagcagagcccatggaacccgagagccggcctggaccctcgggaatgaatgttgtacaggtggctgaactgtatccagaactgagacgcattttgacaattacagaggatgggcaggggctaaagggggtaaagagggagcggggggcttgtgaggctacagaggaggctaggaatctagcttttagcttaatgaccagacaccgtcctgagtgtattacttttcaacagatcaaggataattgcgctaatgagcttgatctgctggcgcagaagtattccatagagcagctgaccacttactggctgcagccaggggatgattttgaggaggctattagggtatatgcaaaggtggcacttaggccagattgcaagtacaagatcagcaaacttgtaaatatcaggaattgttgctacatttctgggaacggggccgaggtggagatagatacggaggatagggtggcctttagatgtagcatgataaatatgtggccgggggtgcttggcatggacggggtggttattatgaatgtaaggtttactggccccaattttagcggtacggttttcctggccaataccaaccttatcctacacggtgtaagcttctatgggtttaacaatacctgtgtggaagcctggaccgatgtaagggttcggggctgtgccttttactgctgctggaagggggtggtgtgtcgccccaaaagcagggcttcaattaagaaatgcctctttgaaaggtgtaccttgggtatcctgtctgagggtaactccagggtgcgccacaatgtggcctccgactgtggttgcttcatgctagtgaaaagcgtggctgtgattaagcataacatggtatgtggcaactgcgaggacagggcctctcagatgctgacctgctcggacggcaactgtcacctgctgaagaccattcacgtagccagccactctcgcaaggcctggccagtgtttgagcataacatactgacccgctgttccttgcatttgggtaacaggaggggggtgttcctaccttaccaatgcaatttgagtcacactaagatattgcttgagcccgagagcatgtccaaggtgaacctgaacggggtgtttgacatgaccatgaagatctggaaggtgctgaggtacgatgagacccgcaccaggtgcagaccctgcgagtgtggcggtaaacatattaggaaccagcctgtgatgctggatgtgaccgaggagctgaggcccgatcacttggtgctggcctgcacccgcgctgagtttggctctagcgatgaagatacagattgaggttctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgactcgaagatcttgtactgaaatgtgtgggcgtggcttaagggtgggaaagaatatataaggtgggggtcttatgtagttttgtatctgttttgcagcagccgccgccgccatgagcaccaactcgtttgatggaagcattgtgagctcatatttgacaacgcgcatgcccccatgggccggggtgcgtcagaatgtgatgggctccagcattgatggtcgccccgtcctgcccgcaaactctactaccttgacctacgagaccgtgtctggaacgccgttggagactgcagcctccgccgccgcttcagccgctgcagccaccgcccgcgggattgtgactgactttgctttcctgagcccgcttgcaagcagtgcagcttcccgttcatccgcccgcgatgacaagttgacggctcttttggcacaattggattctttgacccgggaacttaatgtcgtttctcagcagctgttggatctgcgccagcaggtttctgccctgaaggcttcctcccctcccaatgcggtttaaaacataaataaaaaaccagactctgtttggatttggatcaagcaagtgtcttgctgtctttatttaggggttttgcgcgcgcggtaggcccgggaccagcggtctcggtcgttgagggtcctgtgtattttttccaggacgtggtaaaggtgactctggatgttcagatacatgggcataagcccgtctctggggtggaggtagcaccactgcagagcttcatgctgcggggtggtgttgtagatgatccagtcgtagcaggagcgctgggcgtggtgcctaaaaatgtctttcagtagcaagctgattgccaggggcaggcccttggtgtaagtgtttacaaagcggttaagctgggatgggtgcatacgtggggatatgagatgcatcttggactgtatttttaggttggctatgttcccagccatatccctccggggattcatgttgtgcagaaccaccagcacagtgtatccggtgcacttgggaaatttgtcatgtagcttagaaggaaatgcgtggaagaacttggagacgcccttgtgacctccaagattttccatgcattcgtccataatgatggcaatgggcccacgggcggcggcctgggcgaagatatttctgggatcactaacgtcatagttgtgttccaggatgagatcgtcataggccatttttacaaagcgcgggcggagggtgccagactgcggtataatggttccatccggcccaggggcgtagttaccctcacagatttgcatttcccacgctttgagttcagatggggggatcatgtctacctgcggggcgatgaagaaaacggtttccggggtaggggagatcagctgggaagaaagcaggttcctgagcagctgcgacttaccgcagccggtgggcccgtaaatcacacctattaccgggtgcaactggtagttaagagagctgcagctgccgtcatccctgagcaggggggccacttcgttaagcatgtccctgactcgcatgttttccctgaccaaatccgccagaaggcgctcgccgcccagcgatagcagttcttgcaaggaagcaaagtttttcaacggtttgagaccgtccgccgtaggcatgcttttgagcgtttgaccaagcagttccaggcggtcccacagctcggtcacctgctctacggcatctcgatccagcatatctcctcgtttcgcgggttggggcggctttcgctgtacggcagtagtcggtgctcgtccagacgggccagggtcatgtctttccacgggcgcagggtcctcgtcagcgtagtctgggtcacggtgaaggggtgcgctccgggctgcgcgctggccagggtgcgcttgaggctggtcctgctggtgctgaagcgctgccggtcttcgccctgcgcgtcggccaggtagcatttgaccatggtgtcatagtccagcccctccgcggcgtggcccttggcgcgcagcttgcccttggaggaggcgccgcacgaggggcagtgcagacttttgagggcgtagagcttgggcgcgagaaataccgattccggggagtaggcatccgcgccgcaggccccgcagacggtctcgcattccacgagccaggtgagctctggccgttcggggtcaaaaaccaggtttcccccatgctttttgatgcgtttcttacctctggtttccatgagccggtgtccacgctcggtgacgaaaaggctgtccgtgtccccgtatacagacttgagaggcctgtcctcgagcggtgttccgcggtcctcctcgtatagaaactcggaccactctgagacaaaggctcgcgtccaggccagcacgaaggaggctaagtgggaggggtagcggtcgttgtccactagggggtccactcgctccagggtgtgaagacacatgtcgccctcttcggcatcaaggaaggtgattggtttgtaggtgtaggccacgtgaccgggtgttcctgaaggggggctataaaagggggtgggggcgcgttcgtcctcactctcttccgcatcgctgtctgcgagggccagctgttggggtgagtactccctctgaaaagcgggcatgacttctgcgctaagattgtcagtttccaaaaacgaggaggatttgatattcacctggcccgcggtgatgcctttgagggtggccgcatccatctggtcagaaaagacaatctttttgttgtcaagcttggtggcaaacgacccgtagagggcgttggacagcaacttggcgatggagcgcagggtttggtttttgtcgcgatcggcgcgctccttggccgcgatgtttagctgcacgtattcgcgcgcaacgcaccgccattcgggaaagacggtggtgcgctcgtcgggcaccaggtgcacgcgccaaccgcggttgtgcagggtgacaaggtcaacgctggtggctacctctccgcgtaggcgctcgttggtccagcagaggcggccgcccttgcgcgagcagaatggcggtagggggtctagctgcgtctcgtccggggggtctgcgtccacggtaaagaccccgggcagcaggcgcgcgtcgaagtagtctatcttgcatccttgcaagtctagcgcctgctgccatgcgcgggcggcaagcgcgcgctcgtatgggttgagtgggggaccccatggcatggggtgggtgagcgcggaggcgtacatgccgcaaatgtcgtaaacgtagaggggctctctgagtattccaagatatgtagggtagcatcttccaccgcggatgctggcgcgcacgtaatcgtatagttcgtgcgagggagcgaggaggtcgggaccgaggttgctacgggcgggctgctctgctcggaagactatctgcctgaagatggcatgtgagttggatgatatggttggacgctggaagacgttgaagctggcgtctgtgagacctaccgcgtcacgcacgaaggaggcgtaggagtcgcgcagcttgttgaccagctcggcggtgacctgcacgtctagggcgcagtagtccagggtttccttgatgatgtcatacttatcctgtcccttttttttccacagctcgcggttgaggacaaactcttcgcggtctttccagtactcttggatcggaaacccgtcggcctccgaacggtaagagcctagcatgtagaactggttgacggcctggtaggcgcagcatcccttttctacgggtagcgcgtatgcctgcgcggccttccggagcgaggtgtgggtgagcgcaaaggtgtccctgaccatgactttgaggtactggtatttgaagtcagtgtcgtcgcatccgccctgctcccagagcaaaaagtccgtgcgctttttggaacgcggatttggcagggcgaaggtgacatcgttgaagagtatctttcccgcgcgaggcataaagttgcgtgtgatgcggaagggtcccggcacctcggaacggttgttaattacctgggcggcgagcacgatctcgtcaaagccgttgatgttgtggcccacaatgtaaagttccaagaagcgcgggatgcccttgatggaaggcaattttttaagttcctcgtaggtgagctcttcaggggagctgagcccgtgctctgaaagggcccagtctgcaagatgagggttggaagcgacgaatgagctccacaggtcacgggccattagcatttgcaggtggtcgcgaaaggtcctaaactggcgacctatggccattttttctggggtgatgcagtagaaggtaagcgggtcttgttcccagcggtcccatccaaggttcgcggctaggtctcgcgcggcagtcactagaggctcatctccgccgaacttcatgaccagcatgaagggcacgagctgcttcccaaaggcccccatccaagtataggtctctacatcgtaggtgacaaagagacgctcggtgcgaggatgcgagccgatcgggaagaactggatctcccgccaccaattggaggagtggctattgatgtggtgaaagtagaagtccctgcgacgggccgaacactcgtgctggcttttgtaaaaacgtgcgcagtactggcagcggtgcacgggctgtacatcctgcacgaggttgacctgacgaccgcgcacaaggaagcagagtgggaatttgagcccctcgcctggcgggtttggctggtggtcttctacttcggctgcttgtccttgaccgtctggctgctcgaggggagttacggtggatcggaccaccacgccgcgcgagcccaaagtccagatgtccgcgcgcggcggtcggagcttgatgacaacatcgcgcagatgggagctgtccatggtctggagctcccgcggcgtcaggtcaggcgggagctcctgcaggtttacctcgcatagacgggtcagggcgcgggctagatccaggtgatacctaatttccaggggctggttggtggcggcgtcgatggcttgcaagaggccgcatccccgcggcgcgactacggtaccgcgcggcgggcggtgggccgcgggggtgtccttggatgatgcatctaaaagcggtgacgcgggcgagcccccggaggtagggggggctccggacccgccgggagagggggcaggggcacgtcggcgccgcgcgcgggcaggagctggtgctgcgcgcgtaggttgctggcgaacgcgacgacgcggcggttgatctcctgaatctggcgcctctgcgtgaagacgacgggcccggtgagcttgagcctgaaagagagttcgacagaatcaatttcggtgtcgttgacggcggcctggcgcaaaatctcctgcacgtctcctgagttgtcttgataggcgatctcggccatgaactgctcgatctcttcctcctggagatctccgcgtccggctcgctccacggtggcggcgaggtcgttggaaatgcgggccatgagctgcgagaaggcgttgaggcctccctcgttccagacgcggctgtagaccacgcccccttcggcatcgcgggcgcgcatgaccacctgcgcgagattgagctccacgtgccgggcgaagacggcgtagtttcgcaggcgctgaaagaggtagttgagggtggtggcggtgtgttctgccacgaagaagtacataacccagcgtcgcaacgtggattcgttgatatcccccaaggcctcaaggcgctccatggcctcgtagaagtccacggcgaagttgaaaaactgggagttgcgcgccgacacggttaactcctcctccagaagacggatgagctcggcgacagtgtcgcgcacctcgcgctcaaaggctacaggggcctcttcttcttcttcaatctcctcttccataagggcctccccttcttcttcttctggcggcggtgggggaggggggacacggcggcgacgacggcgcaccgggaggcggtcgacaaagcgctcgatcatctccccgcggcgacggcgcatggtctcggtgacggcgcggccgttctcgcgggggcgcagttggaagacgccgcccgtcatgtcccggttatgggttggcggggggctgccatgcggcagggatacggcgctaacgatgcatctcaacaattgttgtgtaggtactccgccgccgagggacctgagcgagtccgcatcgaccggatcggaaaacctctcgagaaaggcgtctaaccagtcacagtcgcaaggtaggctgagcaccgtggcgggcggcagcgggcggcggtcggggttgtttctggcggaggtgctgctgatgatgtaattaaagtaggcggtcttgagacggcggatggtcgacagaagcaccatgtccttgggtccggcctgctgaatgcgcaggcggtcggccatgccccaggcttcgttttgacatcggcgcaggtctttgtagtagtcttgcatgagcctttctaccggcacttcttcttctccttcctcttgtcctgcatctcttgcatctatcgctgcggcggcggcggagtttggccgtaggtggcgccctcttcctcccatgcgtgtgaccccgaagcccctcatcggctgaagcagggctaggtcggcgacaacgcgctcggctaatatggcctgctgcacctgcgtgagggtagactggaagtcatccatgtccacaaagcggtggtatgcgcccgtgttgatggtgtaagtgcagttggccataacggaccagttaacggtctggtgacccggctgcgagagctcggtgtacctgagacgcgagtaagccctcgagtcaaatacgtagtcgttgcaagtccgcaccaggtactggtatcccaccaaaaagtgcggcggcggctggcggtagaggggccagcgtagggtggccggggctccgggggcgagatcttccaacataaggcgatgatatccgtagatgtacctggacatccaggtgatgccggcggcggtggtggaggcgcgcggaaagtcgcggacgcggttccagatgttgcgcagcggcaaaaagtgctccatggtcgggacgctctggccggtcaggcgcgcgcaatcgttgacgctctagaccgtgcaaaaggagagcctgtaagcgggcactcttccgtggtctggtggataaattcgcaagggtatcatggcggacgaccggggttcgagccccgtatccggccgtccgccgtgatccatgcggttaccgcccgcgtgtcgaacccaggtgtgcgacgtcagacaacgggggagtgctccttttggcttccttccaggcgcggcggctgctgcgctagcttttttggccactggccgcgcgcagcgtaagcggttaggctggaaagcgaaagcattaagtggctcgctccctgtagccggagggttattttccaagggttgagtcgcgggacccccggttcgagtctcggaccggccggactgcggcgaacgggggtttgcctccccgtcatgcaagaccccgcttgcaaattcctccggaaacagggacgagccccttttttgcttttcccagatgcatccggtgctgcggcagatgcgcccccctcctcagcagcggcaagagcaagagcagcggcagacatgcagggcaccctcccctcctcctaccgcgtcaggaggggcgacatccgcggttgacgcggcagcagatggtgattacgaacccccgcggcgccgggcccggcactacctggacttggaggagggcgagggcctggcgcggctaggagcgccctctcctgagcggtacccaagggtgcagctgaagcgtgatacgcgtgaggcgtacgtgccgcggcagaacctgtttcgcgaccgcgagggagaggagcccgaggagatgcgggatcgaaagttccacgcagggcgcgagctgcggcatggcctgaatcgcgagcggttgctgcgcgaggaggactttgagcccgacgcgcgaaccgggattagtcccgcgcgcgcacacgtggcggccgccgacctggtaaccgcatacgagcagacggtgaaccaggagattaactttcaaaaaagctttaacaaccacgtgcgtacgcttgtggcgcgcgaggaggtggctataggactgatgcatctgtgggactttgtaagcgcgctggagcaaaacccaaatagcaagccgctcatggcgcagctgttccttatagtgcagcacagcagggacaacgaggcattcagggatgcgctgctaaacatagtagagcccgagggccgctggctgctcgatttgataaacatcctgcagagcatagtggtgcaggagcgcagcttgagcctggctgacaaggtggccgccatcaactattccatgcttagcctgggcaagttttacgcccgcaagatataccataccccttacgttcccatagacaaggaggtaaagatcgaggggttctacatgcgcatggcgctgaaggtgcttaccttgagcgacgacctgggcgtttatcgcaacgagcgcatccacaaggccgtgagcgtgagccggcggcgcgagctcagcgaccgcgagctgatgcacagcctgcaaagggccctggctggcacgggcagcggcgatagagaggccgagtcctactttgacgcgggcgctgacctgcgctgggccccaagccgacgcgccctggaggcagctggggccggacctgggctggcggtggcacccgcgcgcgctggcaacgtcggcggcgtggaggaatatgacgaggacgatgagtacgagccagaggacggcgagtactaagcggtgatgtttctgatcagatgatgcaagacgcaacggacccggcggtgcgggcggcgctgcagagccagccgtccggccttaactccacggacgactggcgccaggtcatggaccgcatcatgtcgctgactgcgcgcaatcctgacgcgttccggcagcagccgcaggccaaccggctctccgcaattctggaagcggtggtcccggcgcgcgcaaaccccacgcacgagaaggtgctggcgatcgtaaacgcgctggccgaaaacagggccatccggcccgacgaggccggcctggtctacgacgcgctgcttcagcgcgtggctcgttacaacagcggcaacgtgcagaccaacctggaccggctggtgggggatgtgcgcgaggccgtggcgcagcgtgagcgcgcgcagcagcagggcaacctgggctccatggttgcactaaacgccttcctgagtacacagcccgccaacgtgccgcggggacaggaggactacaccaactttgtgagcgcactgcggctaatggtgactgagacaccgcaaagtgaggtgtaccagtctgggccagactattttttccagaccagtagacaaggcctgcagaccgtaaacctgagccaggctttcaaaaacttgcaggggctgtggggggtgcgggctcccacaggcgaccgcgcgaccgtgtctagcttgctgacgcccaactcgcgcctgttgctgctgctaatagcgcccttcacggacagtggcagcgtgtcccgggacacatacctaggtcacttgctgacactgtaccgcgaggccataggtcaggcgcatgtggacgagcatactttccaggagattacaagtgtcagccgcgcgctggggcaggaggacacgggcagcctggaggcaaccctaaactacctgctgaccaaccggcggcagaagatcccctcgttgcacagtttaaacagcgaggaggagcgcattttgcgctacgtgcagcagagcgtgagccttaacctgatgcgcgacggggtaacgcccagcgtggcgctggacatgaccgcgcgcaacatggaaccgggcatgtatgcctcaaaccggccgtttatcaaccgcctaatggactacttgcatcgcgcggccgccgtgaaccccgagtatttcaccaatgccatcttgaacccgcactggctaccgccccctggtttctacaccgggggattcgaggtgcccgagggtaacgatggattcctctgggacgacatagacgacagcgtgttttccccgcaaccgcagaccctgctagagttgcaacagcgcgagcaggcagaggcggcgctgcgaaaggaaagcttccgcaggccaagcagcttgtccgatctaggcgctgcggccccgcggtcagatgctagtagcccatttccaagcttgatagggtctcttaccagcactcgcaccacccgcccgcgcctgctgggcgaggaggagtacctaaacaactcgctgctgcagccgcagcgcgaaaaaaacctgcctccggcatttcccaacaacgggatagagagcctagtggacaagatgagtagatggaagacgtacgcgcaggagcacagggacgtgccaggcccgcgcccgcccacccgtcgtcaaaggcacgaccgtcagcggggtctggtgtgggaggacgatgactcggcagacgacagcagcgtcctggatttgggagggagtggcaacccgtttgcgcaccttcgccccaggctggggagaatgttttaaaaaaaaaaaagcatgatgcaaaataaaaaactcaccaaggccatggcaccgagcgttggttttcttgtattccccttagtatgcggcgcgcggcgatgtatgaggaaggtcctcctccctcctacgagagtgtggtgagcgcggcgccagtggcggcggcgctgggttctcccttcgatgctcccctggacccgccgtttgtgcctccgcggtacctgcggcctaccggggggagaaacagcatccgttactctgagttggcacccctattcgacaccacccgtgtgtacctggtggacaacaagtcaacggatgtggcatccctgaactaccagaacgaccacagcaactttctgaccacggtcattcaaaacaatgactacagcccgggggaggcaagcacacagaccatcaatcttgacgaccggtcgcactggggcggcgacctgaaaaccatcctgcataccaacatgccaaatgtgaacgagttcatgtttaccaataagtttaaggcgcgggtgatggtgtcgcgcttgcctactaaggacaatcaggtggagctgaaatacgagtgggtggagttcacgctgcccgagggcaactactccgagaccatgaccatagaccttatgaacaacgcgatcgtggagcactacttgaaagtgggcagacagaacggggttctggaaagcgacatcggggtaaagtttgacacccgcaacttcagactggggtttgaccccgtcactggtcttgtcatgcctggggtatatacaaacgaagccttccatccagacatcattttgctgccaggatgcggggtggacttcacccacagccgcctgagcaacttgttgggcatccgcaagcggcaacccttccaggagggctttaggatcacctacgatgatctggagggtggtaacattcccgcactgttggatgtggacgcctaccaggcgagcttgaaagatgacaccgaacagggcgggggtggcgcaggcggcagcaacagcagtggcagcggcgcggaagagaactccaacgcggcagccgcggcaatgcagccggtggaggacatgaacgatcatgccattcgcggcgacacctttgccacacgggctgaggagaagcgcgctgaggccgaagcagcggccgaagctgccgcccccgctgcgcaacccgaggtcgagaagcctcagaagaaaccggtgatcaaacccctgacagaggacagcaagaaacgcagttacaacctaataagcaatgacagcaccttcacccagtaccgcagctggtaccttgcatacaactacggcgaccctcagaccggaatccgctcatggaccctgctttgcactcctgacgtaacctgcggctcggagcaggtctactggtcgttgccagacatgatgcaagaccccgtgaccttccgctccacgcgccagatcagcaactttccggtggtgggcgccgagctgttgcccgtgcactccaagagcttctacaacgaccaggccgtctactcccaactcatccgccagtttacctctctgacccacgtgttcaatcgctttcccgagaaccagattttggcgcgcccgccagcccccaccatcaccaccgtcagtgaaaacgttcctgctctcacagatcacgggacgctaccgctgcgcaacagcatcggaggagtccagcgagtgaccattactgacgccagacgccgcacctgcccctacgtttacaaggccctgggcatagtctcgccgcgcgtcctatcgagccgcactttttgagcaagcatgtccatccttatatcgcccagcaataacacaggctggggcctgcgcttcccaagcaagatgtttggcggggccaagaagcgctccgaccaacacccagtgcgcgtgcgcgggcactaccgcgcgccctggggcgcgcacaaacgcggccgcactgggcgcaccaccgtcgatgacgccatcgacgcggtggtggaggaggcgcgcaactacacgcccacgccgccaccagtgtccacagtggacgcggccattcagaccgtggtgcgcggagcccggcgctatgctaaaatgaagagacggcggaggcgcgtagcacgtcgccaccgccgccgacccggcactgccgcccaacgcgcggcggcggccctgcttaaccgcgcacgtcgcaccggccgacgggcggccatgcgggccgctcgaaggctggccgcgggtattgtcactgtgccccccaggtccaggcgacgagcggccgccgcagcagccgcggccattagtgctatgactcagggtcgcaggggcaacgtgtattgggtgcgcgactcggttagcggcctgcgcgtgcccgtgcgcacccgccccccgcgcaactagattgcaagaaaaaactacttagactcgtactgttgtatgtatccagcggcggcggcgcgcaacgaagctatgtccaagcgcaaaatcaaagaagagatgctccaggtcatcgcgccggagatctatggccccccgaagaaggaagagcaggattacaagccccgaaagctaaagcgggtcaaaaagaaaaagaaagatgatgatgatgaacttgacgacgaggtggaactgctgcacgctaccgcgcccaggcgacgggtacagtggaaaggtcgacgcgtaaaacgtgttttgcgacccggcaccaccgtagtctttacgcccggtgagcgctccacccgcacctacaagcgcgtgtatgatgaggtgtacggcgacgaggacctgcttgagcaggccaacgagcgcctcggggagtttgcctacggaaagcggcataaggacatgctggcgttgccgctggacgagggcaacccaacacctagcctaaagcccgtaacactgcagcaggtgctgcccgcgcttgcaccgtccgaagaaaagcgcggcctaaagcgcgagtctggtgacttggcacccaccgtgcagctgatggtacccaagcgccagcgactggaagatgtcttggaaaaaatgaccgtggaacctgggctggagcccgaggtccgcgtgcggccaatcaagcaggtggcgccgggactgggcgtgcagaccgtggacgttcagatacccactaccagtagcaccagtattgccaccgccacagagggcatggagacacaaacgtccccggttgcctcagcggtggcggatgccgcggtgcaggcggtcgctgcggccgcgtccaagacctctacggaggtgcaaacggacccgtggatgtttcgcgtttcagccccccggcgcccgcgcggttcgaggaagtacggcgccgccagcgcgctactgcccgaatatgccctacatccttccattgcgcctacccccggctatcgtggctacacctaccgccccagaagacgagcaactacccgacgccgaaccaccactggaacccgccgccgccgtcgccgtcgccagcccgtgctggccccgatttccgtgcgcagggtggctcgcgaaggaggcaggaccctggtgctgccaacagcgcgctaccaccccagcatcgtttaaaagccggtctttgtggttcttgcagatatggccctcacctgccgcctccgtttcccggtgccgggattccgaggaagaatgcaccgtaggaggggcatggccggccacggcctgacgggcggcatgcgtcgtgcgcaccaccggcggcggcgcgcgtcgcaccgtcgcatgcgcggcggtatcctgcccctccttattccactgatcgccgcggcgattggcgccgtgcccggaattgcatccgtggccttgcaggcgcagagacactgattaaaaacaagttgcatgtggaaaaatcaaaataaaaagtctggactctcacgctcgcttggtcctgtaactattttgtagaatggaagacatcaactttgcgtctctggccccgcgacacggctcgcgcccgttcatgggaaactggcaagatatcggcaccagcaatatgagcggtggcgccttcagctggggctcgctgtggagcggcattaaaaatttcggttccaccgttaagaactatggcagcaaggcctggaacagcagcacaggccagatgctgagggataagttgaaagagcaaaatttccaacaaaaggtggtagatggcctggcctctggcattagcggggtggtggacctggccaaccaggcagtgcaaaataagattaacagtaagcttgatccccgccctcccgtagaggagcctccaccggccgtggagacagtgtctccagaggggcgtggcgaaaagcgtccgcgccccgacagggaagaaactctggtgacgcaaatagacgagcctccctcgtacgaggaggcactaaagcaaggcctgcccaccacccgtcccatcgcgcccatggctaccggagtgctgggccagcacacacccgtaacgctggacctgcctccccccgccgacacccagcagaaacctgtgctgccaggcccgaccgccgttgttgtaacccgtcctagccgcgcgtccctgcgccgcgccgccagcggtccgcgatcgttgcggcccgtagccagtggcaactggcaaagcacactgaacagcatcgtgggtctgggggtgcaatccctgaagcgccgacgatgcttctgaatagctaacgtgtcgtatgtgtgtcatgtatgcgtccatgtcgccgccagaggagctgctgagccgccgcgcgcccgctttccaagatggctaccccttcgatgatgccgcagtggtcttacatgcacatctcgggccaggacgcctcggagtacctgagccccgggctggtgcagtttgcccgcgccaccgagacgtacttcagcctgaataacaagtttagaaaccccacggtggcgcctacgcacgacgtgaccacagaccggtcccagcgtttgacgctgcggttcatccctgtggaccgtgaggatactgcgtactcgtacaaggcgcggttcaccctagctgtgggtgataaccgtgtgctggacatggcttccacgtactttgacatccgcggcgtgctggacaggggccctacttttaagccctactctggcactgcctacaacgccctggctcccaagggtgccccaaatccttgcgaatgggatgaagctgctactgctcttgaaataaacctagaagaagaggacgatgacaacgaagacgaagtagacgagcaagctgagcagcaaaaaactcacgtatttgggcaggcgccttattctggtataaatattacaaaggagggtattcaaataggtgtcgaaggtcaaacacctaaatatgccgataaaacatttcaacctgaacctcaaataggagaatctcagtggtacgaaactgaaattaatcatgcagctgggagagtccttaaaaagactaccccaatgaaaccatgttacggttcatatgcaaaacccacaaatgaaaatggagggcaaggcattcttgtaaagcaacaaaatggaaagctagaaagtcaagtggaaatgcaatttttctcaactactgaggcgaccgcaggcaatggtgataacttgactcctaaagtggtattgtacagtgaagatgtagatatagaaaccccagacactcatatttcttacatgcccactattaaggaaggtaactcacgagaactaatgggccaacaatctatgcccaacaggcctaattacattgcttttagggacaattttattggtctaatgtattacaacagcacgggtaatatgggtgttctggcgggccaagcatcgcagttgaatgctgttgtagatttgcaagacagaaacacagagctttcataccagcttttgcttgattccattggtgatagaaccaggtacttttctatgtggaatcaggctgttgacagctatgatccagatgttagaattattgaaaatcatggaactgaagatgaacttccaaattactgctttccactgggaggtgtgattaatacagagactcttaccaaggtaaaacctaaaacaggtcaggaaaatggatgggaaaaagatgctacagaattttcagataaaaatgaaataagagttggaaataattttgccatggaaatcaatctaaatgccaacctgtggagaaatttcctgtactccaacatagcgctgtatttgcccgacaagctaaagtacagtccttccaacgtaaaaatttctgataacccaaacacctacgactacatgaacaagcgagtggtggctcccgggttagtggactgctacattaaccttggagcacgctggtcccttgactatatggacaacgtcaacccatttaaccaccaccgcaatgctggcctgcgctaccgctcaatgttgctgggcaatggtcgctatgtgcccttccacatccaggtgcctcagaagttctttgccattaaaaacctccttctcctgccgggctcatacacctacgagtggaacttcaggaaggatgttaacatggttctgcagagctccctaggaaatgacctaagggttgacggagccagcattaagtttgatagcatttgcctttacgccaccttcttccccatggcccacaacaccgcctccacgcttgaggccatgcttagaaacgacaccaacgaccagtcctttaacgactatctctccgccgccaacatgctctaccctatacccgccaacgctaccaacgtgcccatatccatcccctcccgcaactgggcggctttccgcggctgggccttcacgcgccttaagactaaggaaaccccatcactgggctcgggctacgacccttattacacctactctggctctataccctacctagatggaaccttttacctcaaccacacctttaagaaggtggccattacctttgactcttctgtcagctggcctggcaatgaccgcctgcttacccccaacgagtttgaaattaagcgctcagttgacggggagggttacaacgttgcccagtgtaacatgaccaaagactggttcctggtacaaatgctagctaactacaacattggctaccagggcttctatatcccagagagctacaaggaccgcatgtactccttctttagaaacttccagcccatgagccgtcaggtggtggatgatactaaatacaaggactaccaacaggtgggcatcctacaccaacacaacaactctggatttgttggctaccttgcccccaccatgcgcgaaggacaggcctaccctgctaacttcccctatccgcttataggcaagaccgcagttgacagcattacccagaaaaagtttctttgcgatcgcaccctttggcgcatcccattctccagtaactttatgtccatgggcgcactcacagacctgggccaaaaccttctctacgccaactccgcccacgcgctagacatgacttttgaggtggatcccatggacgagcccacccttctttatgttttgtttgaagtctttgacgtggtccgtgtgcaccggccgcaccgcggcgtcatcgaaaccgtgtacctgcgcacgcccttctcggccggcaacgccacaacataaagaagcaagcaacatcaacaacagctgccgccatgggctccagtgagcaggaactgaaagccattgtcaaagatcttggttgtgggccatattttttgggcacctatgacaagcgctttccaggctttgtttctccacacaagctcgcctgcgccatagtcaatacggccggtcgcgagactgggggcgtacactggatggcctttgcctggaacccgcactcaaaaacatgctacctctttgagccctttggcttttctgaccagcgactcaagcaggtttaccagtttgagtacgagtcactcctgcgccgtagcgccattgcttcttcccccgaccgctgtataacgctggaaaagtccacccaaagcgtacaggggcccaactcggccgcctgtggactattctgctgcatgtttctccacgcctttgccaactggccccaaactcccatggatcacaaccccaccatgaaccttattaccggggtacccaactccatgctcaacagtccccaggtacagcccaccctgcgtcgcaaccaggaacagctctacagcttcctggagcgccactcgccctacttccgcagccacagtgcgcagattaggagcgccacttctttttgtcacttgaaaaacatgtaaaaataatgtactagagacactttcaataaaggcaaatgcttttatttgtacactctcgggtgattatttacccccacccttgccgtctgcgccgtttaaaaatcaaaggggttctgccgcgcatcgctatgcgccactggcagggacacgttgcgatactggtgtttagtgctccacttaaactcaggcacaaccatccgcggcagctcggtgaagttttcactccacaggctgcgcaccatcaccaacgcgtttagcaggtcgggcgccgatatcttgaagtcgcagttggggcctccgccctgcgcgcgcgagttgcgatacacagggttgcagcactggaacactatcagcgccgggtggtgcacgctggccagcacgctcttgtcggagatcagatccgcgtccaggtcctccgcgttgctcagggcgaacggagtcaactttggtagctgccttcccaaaaagggcgcgtgcccaggctttgagttgcactcgcaccgtagtggcatcaaaaggtgaccgtgcccggtctgggcgttaggatacagcgcctgcataaaagccttgatctgcttaaaagccacctgagcctttgcgccttcagagaagaacatgccgcaagacttgccggaaaactgattggccggacaggccgcgtcgtgcacgcagcaccttgcgtcggtgttggagatctgcaccacatttcggccccaccggttcttcacgatcttggccttgctagactgctccttcagcgcgcgctgcccgttttcgctcgtcacatccatttcaatcacgtgctccttatttatcataatgcttccgtgtagacacttaagctcgccttcgatctcagcgcagcggtgcagccacaacgcgcagcccgtgggctcgtgatgcttgtaggtcacctctgcaaacgactgcaggtacgcctgcaggaatcgccccatcatcgtcacaaaggtcttgttgctggtgaaggtcagctgcaacccgcggtgctcctcgttcagccaggtcttgcatacggccgccagagcttccacttggtcaggcagtagtttgaagttcgcctttagatcgttatccacgtggtacttgtccatcagcgcgcgcgcagcctccatgcccttctcccacgcagacacgatcggcacactcagcgggttcatcaccgtaatttcactttccgcttcgctgggctcttcctcttcctcttgcgtccgcataccacgcgccactgggtcgtcttcattcagccgccgcactgtgcgcttacctcctttgccatgcttgattagcaccggtgggttgctgaaacccaccatttgtagcgccacatcttctctttcttcctcgctgtccacgattacctctggtgatggcgggcgctcgggcttgggagaagggcgcttctttttcttcttgggcgcaatggccaaatccgccgccgaggtcgatggccgcgggctgggtgtgcgcggcaccagcgcgtcttgtgatgagtcttcctcgtcctcggactcgatacgccgcctcatccgcttttttgggggcgcccggggaggcggcggcgacggggacggggacgacacgtcctccatggttgggggacgtcgcgccgcaccgcgtccgcgctcgggggtggtttcgcgctgctcctcttcccgactggccatttccttctcctataggcagaaaaagatcatggagtcagtcgagaagaaggacagcctaaccgccccctctgagttcgccaccaccgcctccaccgatgccgccaacgcgcctaccaccttccccgtcgaggcacccccgcttgaggaggaggaagtgattatcgagcaggacccaggttttgtaagcgaagacgacgaggaccgctcagtaccaacagaggataaaaagcaagaccaggacaacgcagaggcaaacgaggaacaagtcgggcggggggacgaaaggcatggcgactacctagatgtgggagacgacgtgctgttgaagcatctgcagcgccagtgcgccattatctgcgacgcgttgcaagagcgcagcgatgtgcccctcgccatagcggatgtcagccttgcctacgaacgccacctattctcaccgcgcgtaccccccaaacgccaagaaaacggcacatgcgagcccaacccgcgcctcaacttctaccccgtatttgccgtgccagaggtgcttgccacctatcacatctttttccaaaactgcaagatacccctatcctgccgtgccaaccgcagccgagcggacaagcagctggccttgcggcagggcgctgtcatacctgatatcgcctcgctcaacgaagtgccaaaaatctttgagggtcttggacgcgacgagaagcgcgcggcaaacgctctgcaacaggaaaacagcgaaaatgaaagtcactctggagtgttggtggaactcgagggtgacaacgcgcgcctagccgtactaaaacgcagcatcgaggtcacccactttgcctacccggcacttaacctaccccccaaggtcatgagcacagtcatgagtgagctgatcgtgcgccgtgcgcagcccctggagagggatgcaaatttgcaagaacaaacagaggagggcctacccgcagttggcgacgagcagctagcgcgctggcttcaaacgcgcgagcctgccgacttggaggagcgacgcaaactaatgatggccgcagtgctcgttaccgtggagcttgagtgcatgcagcggttctttgctgacccggagatgcagcgcaagctagaggaaacattgcactacacctttcgacagggctacgtacgccaggcctgcaagatctccaacgtggagctctgcaacctggtctcctaccttggaattttgcacgaaaaccgccttgggcaaaacgtgcttcattccacgctcaagggcgaggcgcgccgcgactacgtccgcgactgcgtttacttatttctatgctacacctggcagacggccatgggcgtttggcagcagtgcttggaggagtgcaacctcaaggagctgcagaaactgctaaagcaaaacttgaaggacctatggacggccttcaacgagcgctccgtggccgcgcacctggcggacatcattttccccgaacgcctgcttaaaaccctgcaacagggtctgccagacttcaccagtcaaagcatgttgcagaactttaggaactttatcctagagcgctcaggaatcttgcccgccacctgctgtgcacttcctagcgactttgtgcccattaagtaccgcgaatgccctccgccgctttggggccactgctaccttctgcagctagccaactaccttgcctaccactctgacataatggaagacgtgagcggtgacggtctactggagtgtcactgtcgctgcaacctatgcaccccgcaccgctccctggtttgcaattcgcagctgcttaacgaaagtcaaattatcggtacctttgagctgcagggtccctcgcctgacgaaaagtccgcggctccggggttgaaactcactccggggctgtggacgtcggcttaccttcgcaaatttgtacctgaggactaccacgcccacgagattaggttctacgaagaccaatcccgcccgccaaatgcggagcttaccgcctgcgtcattacccagggccacattcttggccaattgcaagccatcaacaaagcccgccaagagtttctgctacgaaagggacggggggtttacttggacccccagtccggcgaggagctcaacccaatccccccgccgccgcagccctatcagcagcagccgcgggcccttgcttcccaggatggcacccaaaaagaagctgcagctgccgccgccacccacggacgaggaggaatactgggacagtcaggcagaggaggttttggacgaggaggaggaggacatgatggaagactgggagagcctagacgaggaagcttccgaggtcgaagaggtgtcagacgaaacaccgtcaccctcggtcgcattcccctcgccggcgccccagaaatcggcaaccggttccagcatggctacaacctccgctcctcaggcgccgccggcactgcccgttcgccgacccaaccgtagatgggacaccactggaaccagggccggtaagtccaagcagccgccgccgttagcccaagagcaacaacagcgccaaggctaccgctcatggcgcgggcacaagaacgccatagttgcttgcttgcaagactgtgggggcaacatctccttcgcccgccgctttcttctctaccatcacggcgtggccttcccccgtaacatcctgcattactaccgtcatctctacagcccatactgcaccggcggcagcggcagcggcagcaacagcagcggccacacagaagcaaaggcgaccggatagcaagactctgacaaagcccaagaaatccacagcggcggcagcagcaggaggaggagcgctgcgtctggcgcccaacgaacccgtatcgacccgcgagcttagaaacaggatttttcccactctgtatgctatatttcaacagagcaggggccaagaacaagagctgaaaataaaaaacaggtctctgcgatccctcacccgcagctgcctgtatcacaaaagcgaagatcagcttcggcgcacgctggaagacgcggaggctctcttcagtaaatactgcgcgctgactcttaaggactagtttcgcgccctttctcaaatttaagcgcgaaaactacgtcatctccagcggccacacccggcgccagcacctgtcgtcagcgccattatgagcaaggaaattcccacgccctacatgtggagttaccagccacaaatgggacttgcggctggagctgcccaagactactcaacccgaataaactacatgagcgcgggaccccacatgatatcccgggtcaacggaatccgcgcccaccgaaaccgaattctcttggaacaggcggctattaccaccacacctcgtaataaccttaatccccgtagttggcccgctgccctggtgtaccaggaaagtcccgctcccaccactgtggtacttcccagagacgcccaggccgaagttcagatgactaactcaggggcgcagcttgcgggcggctttcgtcacagggtgcggtcgcccgggcagggtataactcacctgacaatcagagggcgaggtattcagctcaacgacgagtcggtgagctcctcgcttggtctccgtccggacgggacatttcagatcggcggcgccggccgtccttcattcacgcctcgtcaggcaatcctaactctgcagacctcgtcctctgagccgcgctctggaggcattggaactctgcaatttattgaggagtttgtgccatcggtctactttaaccccttctcgggacctcccggccactatccggatcaatttattcctaactttgacgcggtaaaggactcggcggacggctacgactgattattaagtggagaggcagagcaactgcgcctgaaacacctggtccactgtcgccgccacaagtgctttgcccgcgactccggtgagttttgctactttgaattgcccgaggatcatatcgaggatctttgttgccatctctgtgctgagtataataaatacagaaattaaaatatactggggctcctatcgccatcctgtaaacgccaccgtcttcacccgcccaagcaaaccaaggcgaaccttacctggtacttttaacatctctccctctgtgatttacaacagtttcaacccagacggagtgagtctacgagagaacctctccgagctcagctactccatcagaaaaaacaccaccctccttacctgccgggaacgtacccttatataaaagtcaggcttcctggtgattaaatgagaattttaattcgaatttaaatgaattcgagctcggtacccggggatcgatccgcctccgcacccgccctcatggaggccgtggagaccggggaacggcccaccttcggagcctgggactacggggtctttgccctcatgctcctggtgtccactggcatcgggctgtgggtcgggctggctcggggcgggcagcgcagcgctgaggacttcttcaccgggggccggcgcctggcggccctgcccgtgggcctgtcgctgtctgccagcttcatgtcggccgtgcaggtgctgggcgtgccgtcggaggcctatcgctatggcctcaagttcctctggatgtgcctgggccagcttctgaactcggtcctcaccgccctgctcttcatgcccgtcttctaccgcctgggcctcaccagcacctacgagtacctggagatgcgcttcagccgcgcagtgcggctctgcgggactttgcagtacattgtagccacgatgctgtacaccggcatcgtaatctacgcaccggccctcatcctgaaccaagtgaccgggctggacatctgggcgtcgctcctgtccaccggaattatctgcaccttctacacggctgtgggcggcatgaaggctgtggtctggactgatgtgttccaggtcgtggtgatgctaagtggcttctgggttgtcctggcacgcggtgtcatgcttgtgggcgggccccgncaggtgctcacgctggnccagaaccactcccggatcaacctcatggactttaaccctgacccgaggagccgctatncattctggacttttgtggtgggtggcacgttggtgtggctctccatgtatggcgtgaaccangcgcaggtgcagcgctacntggcttgccgcacagagaagcaggccaagctggccctgctcatcaaccaggtcggcctgttcctgatcgtgtccagcgctgcctgctgtggcatcgtcatgtttgtgttctacactgactgcgaccctctcctcctggggcgcatctctgccccagaccagtacatgcctctgctggtgctggacatcttcgaagatctgcctggagtccccgggcttttcctggcctgtgcttacagtggcaccctcagcacagcatccaccagcatcaatgctatggctgcagtcactgtagaagacctcatcaaacctcggctgcggagcctggcacccaggaaactcgtgattatctccaaggggctctcactcatctacggatcggcctgtctcaccgtggcagccctgtcctcactgctcggaggaggtgtccttcagggctccttcaccgtcatgggagtcatcagcggccccctgctgggagccttcatcttgggaatgttcctgccggcctgcaacacaccgggcgtcctcgcgggactaggcgcgggcttggcgctgtcgctgtgggtggccttgggcgccacgctgtacccacccagcgagcagaccatgagggtcctgccatcgtcggctgcccgctgcgnggctctctcagtcaacgcctctggcctcctggacccggctctcctccctgctaacgactccagcagggcccccagctcaggaatggacgccagccgacccgcttagctgacagcttctatgccatctcctatctctattacggtgccctgggcacgctgaccactgtgctgtgcggagccctcatcagctgcctgacaggccccaccaagcgcagcaccctggccccgggattgttgtggtgggacctcgcacggcagacagcatcagtggcccccaaggaagaagtggccatcctggatgacaacttggtcaagggtcctgaagaactccccactggaaacaagaagccccctggcttcctgcccaccaatgaggatcgtctgtttttcttggggcagaaggagctggagggggctggctcttggaccccctgtgttggacatgatggtggtcgagaccagcaggagacaaacctctgaggacagggccagccgcgggactgacaccctgggatggagaattctagagtcgacctgcaggaaatggaatttctgtccagtttattcagcagcacctccttgccctcctcccagctctggtattgcagcttcctcctggctgcaaactttctccacaatctaaatggaatgtcagtttcctcctgttcctgtccatccgcacccactatcttcatgttgttgcagatgaagcgcgcaagaccgtctgaagataccttcaaccccgtgtatccatatgacacggaaaccggtcctccaactgtgccttttcttactcctccctttgtatcccccaatgggtttcaagagagtccccctggggtactctctttgcgcctatccgaacctctagttacctccaatggcatgcttgcgctcaaaatgggcaacggcctctctctggacgaggccggcaaccttacctcccaaaatgtaaccactgtgagcccacctctcaaaaaaaccaagtcaaacataaacctggaaatatctgcacccctcacagttacctcagaagccctaactgtggctgccgccgcacctctaatggtcgcgggcaacacactcaccatgcaatcacaggccccgctaaccgtgcacgactccaaacttagcattgccacccaaggacccctcacagtgtcagaaggaaagctagccctgcaaacatcaggccccctcaccaccaccgatagcagtacccttactatcactgcctcaccccctctaactactgccactggtagcttgggcattgacttgaaagagcccatttatacacaaaatggaaaactaggactaaagtacggggctcctttgcatgtaacagacgacctaaacactttgaccgtagcaactggtccaggtgtgactattaataatacttccttgcaaactaaagttactggagccttgggttttgattcacaaggcaatatgcaacttaatgtagcaggaggactaaggattgattctcaaaacagacgccttatacttgatgttagttatccgtttgatgctcaaaaccaactaaatctaagactaggacagggccctctttttataaactcagcccacaacttggatattaactacaacaaaggcctttacttgtttacagcttcaaacaattccaaaaagcttgaggttaacctaagcactgccaaggggttgatgtttgacgctacagccatagccattaatgcaggagatgggcttgaatttggttcacctaatgcaccaaacacaaatcccctcaaaacaaaaattggccatggcctagaatttgattcaaacaaggctatggttcctaaactaggaactggccttagttttgacagcacaggtgccattacagtaggaaacaaaaataatgataagctaaccctatggacaggtccaaaaccagaagccaactgcataattgaatacgggaaacaaaacccagatagcaaactaactttaatccttgtaaaaaatggaggaattgttaatggatatgtaacgctaatgggagcctcagactacgttaacaccttatttaaaaacaaaaatgtctccattaatgtagaactatactttgatgccactggtcatatattaccagactcatcttctcttaaaacagatctagaactaaaatacaagcaaaccgctgactttagtgcaagaggttttatgccaagtactacagcgtatccatttgtccttcctaatgcgggaacacataatgaaaattatatttttggtcaatgctactacaaagcaagcgatggtgccctttttccgttggaagttactgttatgcttaataaacgcctgccagatagtcgcacatcctatgttatgacttttttatggtccttgaatgctggtctagctccagaaactactcaggcaaccctcataacctccccatttaccttttcctatattagagaagatgactaataaactctaaagaatcgtttgtgttatgtttcaacgtgtttatttttcaattgcagaaaatttcaagtcatttttcattcagtagtatagccccaccaccacatagcttatacagatcaccgtaccttaatcaaactcacagaaccctagtattcaacctgccacctccctcccaacacacagagtacacagtcctttctccccggctggccttaaaaagcatcatatcatgggtaacagacatattcttaggtgttatattccacacggtttcctgtcgagccaaacgctcatcagtgatattaataaactccccgggcagctcacttaagttcatgtcgctgtccagctgctgagccacaggctgctgtccaacttgcggttgcttaacgggcggcgaaggagaagtccacgcctacatgggggtagagtcataatcgtgcatcaggatagggcggtggtgctgcagcagcgcgcgaataaactgctgccgccgccgctccgtcctgcaggaatacaacatggcagtggtctcctcagcgatgattcgcaccgcccgcagcataaggcgccttgtcctccgggcacagcagcgcaccctgatctcacttaaatcagcacagtaactgcagcacagcaccacaatattgttcaaaatcccacagtgcaaggcgctgtatccaaagctcatggcggggaccacagaacccacgtggccatcataccacaagcgcaggtagattaagtggcgacccctcataaacacgctggacataaacattacctcttttggcatgttgtaattcaccacctcccggtaccatataaacctctgattaaacatggcgccatccaccaccatcctaaaccagctggccaaaacctgcccgccggctatacactgcagggaaccgggactggaacaatgacagtggagagcccaggactcgtaaccatggatcatcatgctcgtcatgatatcaatgttggcacaacacaggcacacgtgcatacacttcctcaggattacaagctcctcccgcgttagaaccatatcccagggaacaacccattcctgaatcagcgtaaatcccacactgcagggaagacctcgcacgtaactcacgttgtgcattgtcaaagtgttacattcgggcagcagcggatgatcctccagtatggtagcgcgggtttctgtctcaaaaggaggtagacgatccctactgtacggagtgcgccgagacaaccgagatcgtgttggtcgtagtgtcatgccaaatggaacgccggacgtagtcatatttcctgaagcaaaaccaggtgcgggcgtgacaaacagatctgcgtctccggtctcgccgcttagatcgctctgtgtagtagttgtagtatatccactctctcaaagcatccaggcgccccctggcttcgggttctatgtaaactccttcatgcgccgctgccctgataacatccaccaccgcagaataagccacacccagccaacctacacattcgttctgcgagtcacacacgggaggagcgggaagagctggaagaaccatgtttttttttttattccaaaagattatccaaaacctcaaaatgaagatctattaagtgaacgcgctcccctccggtggcgtggtcaaactctacagccaaagaacagataatggcatttgtaagatgttgcacaatggcttccaaaaggcaaacggccctcacgtccaagtggacgtaaaggctaaacccttcagggtgaatctcctctataaacattccagcaccttcaaccatgcccaaataattctcatctcgccaccttctcaatatatctctaagcaaatcccgaatattaagtccggccattgtaaaaatctgctccagagcgccctccaccttcagcctcaagcagcgaatcatgattgcaaaaattcaggttcctcacagacctgtataagattcaaaagcggaacattaacaaaaataccgcgatcccgtaggtcccttcgcagggccagctgaacataatcgtgcaggtctgcacggaccagcgcggccacttccccgccaggaaccatgacaaaagaacccacactgattatgacacgcatactcggagctatgctaaccagcgtagccccgatgtaagcttgttgcatgggcggcgatataaaatgcaaggtgctgctcaaaaaatcaggcaaagcctcgcgcaaaaaagaaagcacatcgtagtcatgctcatgcagataaaggcaggtaagctccggaaccaccacagaaaaagacaccatttttctctcaaacatgtctgcgggtttctgcataaacacaaaataaaataacaaaaaaacatttaaacattagaagcctgtcttacaacaggaaaaacaacccttataagcataagacggactacggccatgccggcgtgaccgtaaaaaaactggtcaccgtgattaaaaagcaccaccgacagctcctcggtcatgtccggagtcataatgtaagactcggtaaacacatcaggttgattcacatcggtcagtgctaaaaagcgaccgaaatagcccgggggaatacatacccgcaggcgtagagacaacattacagcccccataggaggtataacaaaattaataggagagaaaaacacataaacacctgaaaaaccctcctgcctaggcaaaatagcaccctcccgctccagaacaacatacagcgcttccacagcggcagccataacagtcagccttaccagtaaaaaagaaaacctattaaaaaaacaccactcgacacggcaccagctcaatcagtcacagtgtaaaaaagggccaagtgcagagcgagtatatataggactaaaaaatgacgtaacggttaaagtccacaaaaaacacccagaaaaccgcacgcgaacctacgcccagaaacgaaagccaaaaaacccacaacttcctcaaatcgtcacttccgttttcccacgttacgtcacttcccattttaagaaaactacaattcccaacacatacaagttactccgccctaaaacctacgtcacccgccccgttcccacgccccgcgccacgtcacaaactccaccccctcattatcatattggcttcaatccaaaataaggtatattattgatgatgttaattaaggatccnnncggtgtgaaataccgcacagatgcgtaaggagaaaataccgcatcaggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgnnnnnnaaaaaggatcttcacctagatccttttcacgtagaaagccagtccgcagaaacggtgctgaccccggatgaatgtcagctactgggctatctggacaagggaaaacgcaagcgcaaagagaaagcaggtagcttgcagtgggcttacatggcgatagctagactgggcggttttatggacagcaagcgaaccggaattgccagctggggcgccctctggtaaggttgggaagccctgcaaagtaaactggatggctttctcgccgccaaggatctgatggcgcaggggatcaagctctgatcaagagacaggatgaggatcgtttcgcatgattgaacaagatggattgcacgcaggttctccggccgcttgggtggagaggctattcggctatgactgggcacaacagacaatcggctgctctgatgccgccgtgttccggctgtcagcgcaggggcgcccggttctttttgtcaagaccgacctgtccggtgccctgaatgaactgcaagacgaggcagcgcggctatcgtggctggccacgacgggcgttccttgcgcagctgtgctcgacgttgtcactgaagcgggaagggactggctgctattgggcgaagtgccggggcaggatctcctgtcatctcaccttgctcctgccgagaaagtatccatcatggctgatgcaatgcggcggctgcatacgcttgatccggctacctgcccattcgaccaccaagcgaaacatcgcatcgagcgagcacgtactcggatggaagccggtcttgtcgatcaggatgatctggacgaagagcatcaggggctcgcgccagccgaactgttcgccaggctcaaggcgagcatgcccgacggcgaggatctcgtcgtgacccatggcgatgcctgcttgccgaatatcatggtggaaaatggccgcttttctggattcatcgactgtggccggctgggtgtggcggaccgctatcaggacatagcgttggctacccgtgatattgctgaagagcttggcggcgaatgggctgaccgcttcctcgtgctttacggtatcgccgctcccgattcgcagcgcatcgccttctatcgccttcttgacgagttcttctgaattttgttaaaatttttgttaaatcagctcattttttaaccaataggccgaaatcggcaacatcccttataaatcaaaagaatagaccgcgatagggttgagtgttgttccagtttggaacaagagtccactattaaagaacgtggactccaacgtcaaagggcgaaaaaccgtctatcagggcgatggcccactacgtgaaccatcacccaaatcaagttttttgcggtcgaggtgccgtaaagctctaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgcgcgcttaatgcgccgctacagggcgcgtccattcgccattcaggatcgaattaattcttaa